(19) United States
(12) Patent Application Publication
(10) Pub. No.: US 2012/0164104 A1
(43) Pub. Date: Jun. 28, 2012

(54) Composition and Methods of Treating Viral Infections and Viral Induced Tumors

(75) Inventors: Ernest Randall Lanier, Chapel Hill, NC (US); George R. Painter, Chapel Hill, NC (US)

(73) Assignee: Chimerix, Inc., Durham, NC (US)

(21) Appl. No.: 13/388,771

(22) PCT Filed: Aug. 2, 2010
(86) PCT No.: PCT/US10/44093

§ 371 Date: Mar. 16, 2012

Related U.S. Application Data
(60) Provisional application No. 61/230,931, filed on Aug. 3, 2009.

(51) Int. Cl. A61K 31/675; A61K 39/395; A61P 31/20; A61K 33/24; A61K 38/21; A61K 39/00; A61P 35/00; A61K 38/13

(52) U.S. Cl. 424/85.5; 514/81; 514/86; 424/133.1; 514/20.5; 424/649; 424/192.1

(57) ABSTRACT

The present invention provides methods of treating viral induced tumor or viral infections, including administering a compound described in the invention in a therapeutically effective amount. According to some aspect of the present invention, the methods may further comprise at least one immunosuppressant agent to treat viral infection and/or viral induced tumor to a subject in need of immunosuppressant agents.

### RELATED APPLICATIONS

This application claims the benefit under 35 U.S.C. §119(e) of U.S. Provisional Patent Application Ser. No. 61/230,931, filed Aug. 3, 2009, the disclosures of which are incorporated herein by reference in its entirety.

### FIELD OF THE INVENTION

The present invention generally relates to compounds, analogues thereof and methods for treating viral infections and virally induced tumors.

### BACKGROUND OF THE INVENTION

Cancers are the result of a disruption of the normal restraints on cellular proliferation. Cancer causes about 13% of all human deaths. According to the American Cancer Society, 7.6 million people died from cancer in the world during 2007 alone. An important cause for cancer is viral infection. It is estimated that viruses are responsible for 15% of human cancers worldwide. Viral infections appear to be the second most important risk factor for cancer development in humans, exceeded only by tobacco usage. The main viruses associated with human cancers are human papillomavirus, hepatitis B and hepatitis C virus, Epstein-Barr virus, and human T-lymphotropic virus.

In addition, patients with immunodeficiency are particularly vulnerable to opportunistic viruses such as polyomavirus (e.g. BK virus or JC virus). Some recent studies have showed that there may a connection between polyomavirus and cancer. (See Feng et al., Science, vol. 39, 1096-1100 (February 2008) and Moens et al., Cell. Mol. Life. Sci., 64, 1656-1678 (2007)). Other research also show that there is a connection between cytomegalovirus and colonic pseudotumor and glioblastoma multiforme (GEM), (See Jesus et al., J. Neurooncol, 94:445-448 (2009), Mitechell, et al., Neuro-oncology, page 10-18 (February, 2008), Epstien-Barr virus and gastric cancer, (See Truong et al., J. of Experimental & Clinical Cancer Research 2009, 28:14), human papilloma virus and cervical cancer (See Schiffman et al, Seminar, Vol. 37, pages 890-907, Sep. 8, 2007, which is available at http: www.thelancet.com).

Therefore, there is a need for new therapies that can be used to treat virus associated tumor, particularly for immunocompromised patients.

### SUMMARY OF THE INVENTION

A first aspect of the invention is methods of treating virally induced conditions/disease associated with immune suppression in a subject (for example, virally induced tumor or viral infection), the method comprising: administering to said subject a therapeutically effective amount of compounds described herein. The compounds described herein are specifically targeted against viral replication and/or virally infected/transformed cells (e.g. virally induced tumor cells).

In some embodiments, the viral induced tumor is associated with polyomavirus (including BK, John Cunningham virus (JCV), Merkel cell virus (MCV), KI polyomavirus (KIV), WU polyomavirus (WUV), Simian virus 40 (SV 40)), papillomavirus (including human papillomavirus, cottontail rabbit papillomavirus, equine papillomavirus and bovine papillomavirus), herpes virus, adenovirus, Epstein-Barr virus (EBV), human cytogegalovirus (HCMV), Hepatitis B virus, Hepatitis C virus or a combination thereof.

In some embodiments, the viral induced tumor is associated with BK or JC virus.

In another embodiment, the subject is immunocompromised. Further, in one embodiment, the subject is a renal transplant or liver transplant patient.

In one embodiment, the compound is

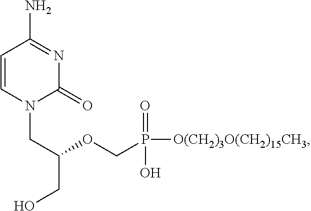

or a pharmaceutically acceptable salt thereof.

A further aspect of the invention is, methods for the therapeutic and/or prophylactic treatment of a viral infection in a subject in need of an immunosuppressant agent which includes administering to said subject a therapeutically effective amount of compound described herein in combination with one or more immunosuppressant agents.

In some embodiments, at least one immunosuppressant agent is selected from Daclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm and a combination thereof.

In one embodiment, at least one immunosuppressant agents is Tysabri (natalizumab).

A further aspect of the invention provides pharmaceutical composition including (a) a compound described herein, (b) at least one immunosuppressant agents, and (c) a pharmaceutically acceptable carrier.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates the effect of increasing concentrations of CMX001 on BKV load and expression of BKV proteins. FIG. 1(a) illustrates the relationship between the concentration of CMX001 and reduction of extracellular BKV load. FIG. 1(b) shows the image of immunofluorescence staining 72 h p.i. of BKV-infected RPTECs.

For FIG. 1(a), RPTEC supernatants were harvested 72 h p.i. i.e. 70 h post start of treatment with indicated CMX001 concentrations. BKV load was measured by OCR and input virus subtracted. DNA load in untreated cells (1.05E+09 Geq/ml) was set as 100%.

For FIG. 1(b), indirect immunofluorescence of untreated and CMX001 treated BKV-infected RPTECs, methanol fixed 72 h pd. and stained with rabbit anti-agnoprotein serum (green) for visualization of the late agnoprotein and with the SV40 LTag monoclonal Pab416 for visualization og BKV LTag (red). Cell density is shown by Drac 5 staining in blue.

FIG. 2(a) illustrates the relationship between the concentration of CMX001 and DNA replication of uninfected RPTEC. FIG. 2(b) illustrates the relationship between the concentration of CMX001 and metabolic activity of uninfected RPTEC.

For FIG. 2(a), cellular DNA replication was examined with BrdU incorporation. Medium with indicated CMX001 concentrations was added 2 h p.i. and absorbance measured 72 h p.i. Absorbance for untreated cells was set as 100%.

For FIG. 2(b) metabolic activity was examined as WST-1 cleavage. Medium with indicated CMX001 concentrations was added 2 h p.i. and absorbance measured 72 h p.i. Absorbance for untreated cells was set as 100%.

FIG. 3 illustrates the influence of CMX001 0.31 μM on BKV genome replication. For Figure, CMX001-treated and untreated BKV-infected RPTECs were harvested at indicated timepoints and intracellular BKV DNA load per cell was measured by qPCR.

FIG. 4 illustrates the influence of CMX001 0.31 μM on BKV early and late expression. FIG. 4(a) shows the image of indirect immunofluorescence of untreated and CMX001 treated BKV-infected RPTECs. FIG. 4(b) shows the cell extracts from CMX001-treated and untreated BKV-infected RPTECs were harvested 48 and 72 h p.i. and western blot performed with rabbit anti-BKV VP1, anti-agnoprotein serum and a monoclonal antibody directed against the housekeeping protein GAPDH.

For FIG. 4(a), indirect immunofluorescence of untreated and CMX001 treated BKV-infected RPTECs, methanol fixed 48 and 72 h p.i. and stained with rabbit anti-agnoprotein serum (green) for visualization of the late agnoprotein and with the SV40 LTag monoclonal Pab416 for visualization of BKV LTag (red). For FIG. 4(b), cell extracts from CMX001-treated and untreated BKV-infected RPTECs were harvested 48 and 72 h p.i. and western blot performed with rabbit anti-BKV VP1, anti-agnoprotein serum and a monoclonal antibody directed against the housekeeping protein GAPDH.

FIG. 5 illustrates the influence of CMX001 0.31 μM on BKV extracellular BKV load. FIG. 5 demonstrates influence of CMX001 0.31 μM on BKV extracellular BKV load. Supernatants from CMX001-treated and untreated BKV-infected RPTECs were harvested at indicated timepoints after infection and BKV load measured by qPCR. Data are presented as BKV load in Geq/ml.

FIG. 6 demonstrates the impact of CMX001 for pre-treatment of RPTECs before infection. For FIG. 6, RPTECs were either treated for 4 hours until 20 h pre-infection when new complete growth medium was added, or they were treated for 24 hours until one hour before infection when they were washed for one hour in complete growth medium before infection. Supernatants were harvested 72 h p.i. and extracellular BKV load measured by qPCR. Data are presented in percent of untreated cells set at 100%.

FIG. 7 illustrates the stability of CMX001. For FIG. 7, BKV-infected RPTECs were treated with freshly made CMX001 or CMX001 from a stock solution at 1 mg/ml stored for one week at 4° C. or −20° C. Supernatants were harvested 72 h p.i. and extracellular BKV load measured by qPCR. Data are presented as BKV load in percent of untreated cells set at 100%.

FIG. 8 demonstrates the replication of JCV Mad-4 in COS-7 cells. For FIG. 8, Indirect immunofluorescence of JCVinfected COS-7 cells, fixed 7 d.p.i. and stained with rabbit anti-VP1 serum (red) for visualization of the late capsid protein VP1 and with Höchst 33342 dye to show DNA (blue). The merged pictures are shown in the right panel.

FIG. 9 shows replication of JCV Mad-4 in astrocyte cells and the image of indirect immunofluorescence of JCV-infected astrocyte cells. Fixation and staining are the same as in FIG. 8.

FIG. 10 demonstrates the course of JCV replication in astrocyte cells. FIG. 10(a) shows the indirect immunofluorescence of JCV-infected astrocyte cells, fixed 7 d.p.i. and stained with rabbit anti-VP1 serum (red) for visualization of the late capsid protein VP1 and with Höchst 33342 dye to show DNA (blue). The merged pictures are shown in the right panel. FIG. 10(b) shows the image of indirect immunofluorescence of JCV infected astrocyte cells, fixed at 14 d.p.i. Fixation and staining as in a). FIG. 10(c) shows the image of indirect immunofluorescence of mock-infected astrocyte cells, fixed at 14 d.p.i. Fixation and staining as in 10(a).

FIG. 11 demonstrates the replication of religated JCV Mad-4 DNA in COS-7 cells and the image of indirect immunofluorescence of JCV DNA transfected COS-7 cells, fixed 7 d.p.t. and stained with rabbit anti-VP1 serum (red) for visualization of the late capsid protein VP1 and with Höchst 33342 dye to show DNA (blue). The merged pictures are shown in the right panel.

FIG. 12 demonstrates that the determination of CMX001IC-50 and IC-90. COS-7 supernatants were harvested 5 d.p.i., i.e. 118 h post start of treatment with indicated CMX001 concentrations. JCV load was measured by qPCR and input virus subtracted. DNA load in untreated cells (5.04×10 E+9 geq/ml) was set as 100%. Replication of JCV is shown as percentage of untreated cells to determine the IC-50 and IC-90.

FIG. 13 demonstrates the effect of increasing concentrations of CMX001 on metabolic activity of COS-7 cells. Metabolic activity was examined as WST-1 cleavage. Medium with indicated CMX001 concentrations was added to COS-7 cells and absorbance measured 72 h post seeding. Absorbance for untreated cells was set as 100%.

FIG. 14 illustrates the effect of increasing concentrations of CMX001 on replication of COS-7 cells. DNA replication was determined by BrdU incorporation. Medium with indicated CMX001 concentrations was added to COS-7 cells and absorbance measured 72 h post seeding. Absorbance for untreated cells was set as 100%.

FIG. 15 shows the effect of increasing concentrations of CMX001 on extracellular viral load. Supernatants from CMX001-treated and untreated JCV-infected COS-7 cells were harvested at indicated timepoints after infection and JCV load measured by qPCR. Data are presented as JCV load in log geq/ml.

FIG. 16 illustrates the effect of increasing concentrations of CMX001 on expression of JCV proteins. Indirect immunofluorescence of JCV-infected COS-7 cells treated with indicated concentrations of CMX001, fixed 7 d.p.i. and stained with rabbit anti-VP1 serum (red) for visualization of the late capsid protein VP1 and with Höchst 33342 dye to show DNA (blue). The merged pictures are shown in the right panel.

FIG. 17 illustrates the effect of increasing concentrations of CMX001 on extracellular viral load in astrocytes. Supernatants from JCV-infected PDA cells treated with indicated concentrations of CMX001 were harvested at indicated timepoints after infection and JCV load measured by qPCR. Data are presented as JCV load in log geq/ml.

FIG. 18 graphically illustrates inhibition of Caki-1 cell growth by Doxorubicin, Cidofovir and CMX001.

FIG. 19 graphically illustrates inhibition of Caki-2 cell growth by Doxorubicin, Cidofovir and CMX001.

FIG. 20 graphically illustrates inhibition of SCaBER cell growth by Doxorubicin, Cidofovir and CMX001.

FIG. 21 graphically illustrates inhibition of SW780 cell growth by Doxorubicin, Cidofovir and CMX001.

FIG. 22 graphically illustrates inhibition of DU 145 cell growth by Docetaxel (A), Cidofovir and CMX001 (B).

FIG. 23 graphically illustrates inhibition of PC-3 cell growth by Docetaxel (A), Cidofovir and CMX001 (B).

FIG. 24 graphically illustrates inhibition of MDA-231 cell growth by Docetaxel (A), Cidofovir and CMX001 (B).

FIG. 25 graphically illustrates inhibition of MCF-7 cell growth by Docetaxel (A), Cidofovir and CMX001 (B).

FIG. 26 graphically illustrates Inhibition of PANC-1 cell growth by Gemcitabine, Cidofovir and CMX001.

FIG. 27 graphically illustrates inhibition of MIA PaCa-2 cell growth by Gemcitabine, Cidofovir and CMX001.

FIG. 28 graphically illustrates inhibition of HT-29 cell growth by Oxaliplatin, Cidofovir and CMX001

FIG. 29 graphically illustrates inhibition of HCT-116 cell growth by Oxaliplatin, Cidofovir and CMX001.

FIG. 30 graphically illustrates inhibition of H460 cell growth by Docetaxel (A), Cidofovir and CMX001 (B).

FIG. 31 graphically illustrates inhibition of A549 cell growth by Docetaxel (A), Cidofovir and CMX001 (B).

FIG. 32 graphically illustrates inhibition of A2058 cell growth by DTIC, Cidofovir and CMX001.

FIG. 33 graphically illustrates inhibition of SK-MEL-2 cell growth by DTIC, Cidofovir and CMX001.

FIG. 34 graphically illustrates inhibition of C33 cell growth by Cisplatin, Cidofovir and CMX001.

FIG. 35 graphically illustrates inhibition of SiHa cell growth by Cisplatin, Cidofovir and CMX001.

FIG. 36 graphically illustrates inhibition of SK-OV-3 cell growth by Cisplatin, Cidofovir and CMX001.

FIG. 37 graphically illustrates inhibition of OvCar-3 cell growth by Cisplatin, Cidofovir and CMX001.

FIG. 38 graphically illustrates inhibition of SK-HEP-1 cell growth by Doxorubicin, Cidofovir and CMX001.

FIG. 39 graphically illustrates inhibition of HEP G2 cell growth by Doxorubicin, Cidofovir and CMX001.

FIG. 40 graphically illustrates inhibition of Het-1A cell growth by Cidofovir and CMX001.

FIG. 41 graphically illustrates inhibition of THLE-3 cell growth by Cidofovir and CMX001.

### DETAILED DESCRIPTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

All patents, patent applications and publications referred to herein are incorporated by reference in their entirety. In case of a conflict in terminology, the present specification is controlling.

As used herein, “alkyl” refers to a straight or branched chain hydrocarbon containing from 1 to 30 carbon atoms. In some embodiments, the alkyl group contains 1 to 24, 2 to 25, 2 to 24, 17 to 20, 1 to 10, or 1 to 8 carbon atoms. In some embodiments the alkyl group contains 17-20 carbon atoms. In some embodiments, the alkyl group contains 17, 18, 19 or 20 carbon atoms. In still other embodiments, alkyl group contains 1-5 carbon atoms, and in yet other embodiments, alkyl group contain 1-4 or 1-3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. Additional examples or generally applicable substituents are illustrated by the specific embodiments shown in the Examples which are described herein.

As used herein, “alkenyl,” refers to a straight or branched chain hydrocarbon containing from 2 to 30 carbons and containing at least one carbon-carbon double bond formed by the removal of two hydrogens. In some embodiments, the alkenyl group contains 2 to 25, 2 to 24, 17 to 20, 2 to 10, 2 to 8 carbon atoms. In some embodiments, the alkenyl group contains 17-20 carbon atoms. In still other embodiments, alkenyl groups contain 17, 18, 19 or 20 carbon atoms, and in yet other embodiments alkenyl groups contain 2-5, 2-4 or 2-3 carbon atoms. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. Additional examples or generally applicable substituents are illustrated by the specific embodiments shown in the Examples which are described herein.

As used herein, “alkynyl,” refers to a straight or branched chain hydrocarbon group containing from 2 to 30 carbon atoms and containing at least one carbon-carbon triple bond. In some embodiments, the alkynyl group contains 2 to 25, 2 to 24, 2 to 10 or 2 to 8 carbon atoms. In some embodiments, the alkynyl group contains 17-20 carbon atoms. In still other embodiments, alkynyl groups contain 17, 18, 19 or 20 carbon atoms, and in yet other embodiments, alkynyl groups contain 2-5, 2-4 or 2-3 carbon atoms. Representative examples of alkynyl include, but are not limited, to ethynyl, 1-propynyl, 2-propynyl, 3-butyryl, 2-pentynyl, 1-butynyl and the like. Additional examples or generally applicable substituents are illustrated by the specific embodiments shown in the Examples which are described herein.

As used herein, “acyl,” refers to a straight or branched chain hydrocarbon containing from 2 to 30 carbons and at least one carbon of the hydrocarbon chain is substituted with an oxo (═O). In some embodiments, the acyl group contains 2 to 25, 2 to 24, 17 to 20, 2 to 10, 2 to 8 carbon atoms. In some embodiments, the acyl group contains 17-20 carbon atoms. In still other embodiments, the acyl group contains 17, 18, 19 or 20 carbon atoms, and in yet other embodiments, the acyl group contains 2-5, 2-4 or 2-3 carbon atoms. Additional examples or generally applicable substituents are illustrated by the specific embodiments shown in the Examples which are described herein.

As used herein, the term “alkoxy” refers to an alkyl group, as previously defined, attached to the parent molecular moiety through an oxygen atom. In some embodiments the alkoxy group contains 1-30 carbon atoms. In other embodiment, the alkoxy group contains 1-20, 1-10 or 1-5 carbon atoms. In some embodiments, the alkoxy group contains 2 to 25, 2 to 24, 17 to 20, 2 to 10, 2 to 8 carbon atoms. In some embodiments, the alkoxy group contains 17-20 carbon atoms. In still other embodiments, the alkoxy group contains 17, 18, 19 or 20 carbon atoms. In some embodiments, the alkoxyl group contains 1 to 8 carbon atoms. In some embodiments, the alkoxyl group contains 1 to 6 carbon atoms. In some embodiments, the alkoxyl group contains 1 to 4 carbon atoms. In still other embodiments, alkoxyl group contains 1-5 carbon atoms, and in yet other embodiments, alkoxyl group contain 1-4 or 1-3 carbon atoms. Representative examples of alkoxyl include, but are not limited to, methoxy, ethoxy, propoxy, isopropoxy, n-butoxy, tert-butoxy, and n-pentoxy. Additional examples or generally applicable substituents are illustrated by the specific embodiments shown in the Examples which are described herein.

The term “aliphatic”, as used herein, includes saturated, unsaturated, straight chain (i.e., unbranched), or branched, aliphatic hydrocarbons, which are optionally substituted with one or more functional groups. In some embodiments, the aliphatic may contain one or more function groups selected from double bond, triple bond or carbonyl group (C═O), or a combination thereof. As will be appreciated by one of ordinary skill in the art, “aliphatic” is intended herein to include, but is not limited to, alkyl, alkenyl, alkynyl, or acyl moieties. Thus, as used herein, the term “alkyl” includes straight, branched saturated groups. An analogous convention applies to other generic terms such as “alkenyl”, “alkynyl”, “acyl” and the like. Furthermore, as used herein, the terms “alkyl”, “alkenyl”, “alkynyl”, “acyl” and the like encompass both substituted and unsubstituted groups. In some embodiments, the term “aliphatic” refers to —(C₁-C₂₄)alkyl, —(C₂-C₂₄)alkenyl, —(C₂-C₂₄)alkynyl, —(C₁-C₂₄)acyl. As understood by one skilled in the art, the range of carbon number indicated above encompasses individual number within the range.

As used herein, “amino acid residue” refers to a compound consisting of a carbon atom which is bonded to a primary amino (—NH₂) group, a carboxylic acid (—COOH) group, a side chain, and a hydrogen atom. For example, the term “amino acid” includes, but is not limited to, Glycine, Alanine, Valine, Leucine, Isoleucine, Serine, Threonine, Aspartic acid and Glutamic acid. In the present invention, in Formula I or Ia, when R₂ is —NR′H and R′ is an amino acid residue, N is attached to the carbon atom as a side chain. Additionally, as used herein, “amino acid” also includes derivatives of amino acids such as esters, and amides, and salts, as well as other derivatives, including derivatives having pharmacoproperties upon metabolism to an active form. Additional examples or generally applicable substituents are illustrated by the specific embodiments shown in the Examples which are described herein.

As used herein, “cycloalkyl” refers to a monovalent saturated cyclic or bicyclic hydrocarbon group of 3-12 carbons derived from a cycloalkane by the removal of a single hydrogen atom. In some embodiments, cycloalkyl contains 3 to 8 carbon atoms. In some embodiments, cycloalkyl contains 3 to 6 carbon atoms. Cycloalkyl groups may be optionally substituted with alkyl, alkoxy, halo, or hydroxy substituents. Representative examples of cycloalkyl include, but are not limited to, are cyclopropyl, cyclobutyl, cyclopentyl, cyclohexyl, cycloheptyl, and cyclooctyl. Additional examples of generally applicable substituents are illustrated by the specific embodiments shown in the Examples that are described herein.

As used herein, “heteroalkyl,” “heteroalkenyl” or “heteroalkynyl” refer to alkyl, alkenyl or alkynyl groups which contain one or more oxygen, sulfur, nitrogen, phosphorus or silicon atoms, e.g., in place of carbon atoms. In some embodiments, the heteroalkyl group contains 1-8 carbon atoms. In certain embodiments, the heteroalkenyl and heteralkynyl groups independently contain 2-8 carbon atoms. In still other embodiments, heteroalkyl, heteroalkenyl and heteralkynyl independently contain 2-5 carbon atoms, and in yet other embodiments, heteroalkyl, heteroalkenyl and heteralkynyl independently contain 2-4 or 2-3 carbon atoms.

The term “heterocycloalkyl” or “heterocycle”, as used herein, refers to a non-aromatic, saturated or unsaturated, 5-, 6- or 7-membered ring or a polycyclic group, including, but not limited to a bi- or tri-cyclic having between one or more heteroatoms independently selected from oxygen, sulfur and nitrogen as part of the ring, wherein (i) each 5-membered ring has 0 to 1 double bonds and each 6-membered ring has 0 to 2 double bonds, (ii) the nitrogen and sulfur heteroatoms may be optionally oxidized, (iii) the nitrogen heteroatom may optionally be quaternized, and/or (iv) any of the above heterocyclic rings may be fused to a benzene ring. Exemplary heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

As used herein, the term “halogen” refers to fluorine (F), chlorine (Cl), bromine (Br), or iodine (I) and the term “halo” refers to the halogen radicals: fluoro (—F), chloro (—Cl), bromo (—Br), and iodo (—I).

As used herein, the term “haloalkyl” refers to a straight or branched chain alkyl group as defined herein containing at least one carbon atoms substituted with at least one halo group, halo being as defined herein. In some embodiments, the haloalkyl contains 1 to 30 carbon atoms. In some embodiments, the halkalkyl contains 1 to 8 or 1 to 6 carbon atoms. In other embodiments, the haloalkyl contains 1 to 4 carbon atoms. Additional examples or generally applicable substituents are illustrated by the specific embodiments shown in the Examples which are described herein.

As used herein, the term “aryl” refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated. For example, an aryl may be substituted with one or more heteroatoms (e.g., oxygen, sulfur and/or nitrogen). Additional examples or generally applicable substituents are illustrated by the specific embodiments shown in the Examples which are described herein.

In some embodiments, alkyl, alkenyl, alkynyl, alkoxy, cycloalkyl, cycloalkenyl, heterocycloalkyl, aryl, heteroaryl, acyl, described herein include both substituted and unsubstituted moieties. Exemplary substituents include, but are not limited to, halo, hydroxyl, amino, amide, —SH, cyano, nitro, thioalkyl, carboxylic acid, —NH—C(═NH)—NH₂, alkyl, alkenyl, alkynyl, aryl, heteroaryl, cycloalkyl, heterocycloalkyl, in which alkyl, alkenyl, alkynyl, alkoxyl, aryl, heteroaryl, cycloalkyl, and heterocycloalkyl may be further substituted.

As used herein, the term “amino acid” refers to a compound comprising a primary amino (—NH₂) group and a carboxylic acid (—COOH) group. The amino acids used in the present invention include naturally occurring and synthetic α, γ, γ or δ amino acids and L, D amino acid, and includes but are not limited to, amino acids found in proteins. Exemplary amino acids include, but are not limited to, glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In some embodiments, the amino acid may be a derivative of alanyl, valinyl, leucinyl, isoleucinyl, prolinyl, phenylalaninyl, tiyptophanyl, methioninyl, glycinyl, serinyl, threoninyl, cysteinyl, tyrosinyl, asparaginyl, glutaminyl, aspartoyl, glutaroyl, lysinyl, argininyl, histidinyl, alanyl, β-valinyl, β-leucinyl, β-isoleucinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. Additionally, as used herein, “amino acids” also include derivatives of amino acids such as esters, and amides, and salts, as well as other derivatives, including derivatives having pharmacoproperties upon metabolism to an active form.

As used herein, the term “natural a amino acid” refers to a naturally occurring α-amino acid comprising a carbon atom bonded to a primary amino (—NH₂) group, a carboxylic acid (—COOH) group, a side chain, and a hydrogen atom. Exemplary natural a amino acids include, but are not limited to, glycine, alanine, valine, leucine, isoleucine, methionine, phenylalanine, tryptophane, proline, serine, threonine, cysteine, tyrosine, asparaginate, glutaminate, aspartate, glutamate, lysine, arginine and histidine.

As used herein, the term “viral induced tumor” is defined as a tumor that is induced, caused and/or associated with at least one type of virus described herein.

Subjects to be treated by the methods of the present invention are, in general, mammalian and primate subjects (e.g., human, monkey, ape, chimpanzee). Subjects may be male or female and may be of any age, including prenatal (i.e., in utero), neonatal, infant, juvenile, adolescent, adult, and geriatric subjects. Thus, in some cases the subjects may be pregnant female subjects. Treatment may be for any purpose, including the therapeutic treatment of previously infected subjects, as well as the prophylactic treatment of uninfected subjects (e.g., subjects identified as being at high risk for infection).

As used herein, “Human immunodeficiency virus” (or “HIV”) as used herein is intended to include all subtypes thereof, including HIV subtypes A, B, C, D, E, F, G, and O, and HIV-2.

As used herein, “Hepatitis B virus” (or “HBV”) as used herein is intended to include all subtypes (adw, adr, ayw, and ayr) and or genotypes (A, B, C, D, E, F, G, and H) thereof.

As used herein, or “a therapeutically effective amount” refers to an amount that will provide some alleviation, mitigation, and/or decrease in at least one clinical symptom in the subject. Those skilled in the art will appreciate that the therapeutic effects need not be complete or curative, as long as some benefit is provided to the subject.

As used herein, “specificity” or “specifically against” refers to a compound that a compound that may selectively inhibit the metabolic activity and/or DNA replication of a certain type of tumor cells or viral infected cells. The specificity may be tested by using any methods known to one skilled in the art, for example, testing IC₉₀ and/or IC₅₀. In some embodiments, the compounds described herein may have IC₉₀ and/or IC₅₀ against viral infected cells to be at least about three fold lower than the IC₉₀ and/or IC₅₀ against normal (uninfected) cells. In some embodiments, the compounds described herein may have IC₉₀ and/or IC₅₀ against viral infected cells to be about three fold to ten fold lower than the IC₉₀ and/or IC₅₀ against normal (uninfected) cells. In some embodiments, the compounds described herein may have IC₉₀ and/or IC₅₀ against viral infected cells to be at least ten fold lower than the IC₉₀ and/or IC₅₀ against normal (uninfected) cells. In some embodiments, the compounds described herein may have specific cytotoxicity against viral infected and/or transformed cells. The cytotoxicity may be measured by any methods known to one skilled in the art.

Unless otherwise stated, structures depicted herein are meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, the terms “treatment,” “treat,” and “treating” refer to reversing, alleviating, inhibiting the progress of a disease or disorder as described herein, or delaying, eliminating or reducing the incidence or onset of a disorder or disease as described herein, as compared to that which would occur in the absence of the measure taken. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Active compounds of the present invention may optionally be administered in combination (or in conjunction) with other active compounds and/or agents useful in the treatment of viral infections as described herein. The administration of two or more compounds “in combination” or “in conjunction” means that the two compounds are administered closely enough in time to have a combined effect, for example an additive and/or synergistic effect. The two compounds may be administered simultaneously (concurrently) or sequentially or it may be two or more events occurring within a short time period before or after each other. Simultaneous administration may be carried out by mixing the compounds prior to administration, or by administering the compounds at the same point in time but at different anatomic sites or using different routes of administration. In some embodiments, the other antiviral agent may optionally be administered concurrently.

### A. Active Compounds

According to some aspects of the present invention, compounds with a range of biological properties are provided. Compounds described herein have biological activities relevant for the treatment of viral induced tumor.

(1) In some embodiments, the compounds of the present invention have the structure of Formula I:

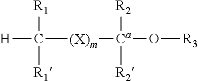

wherein:

R₁, R₁′, R₂ and R₂′ are independently halogen, —ORⁱ, —SRⁱ, —NHRⁱ, —NRⁱRⁱⁱ, and Rⁱ and Rⁱⁱ are independently hydrogen or aliphatic, and

R₃ is a pharmaceutically active phosphonate, bisphosphonate or a phosphonate derivative of a pharmacologically active compound;

X, when present, is:

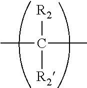

and m is an integer from 0 to 6.

In some embodiments, said alkyl, alkenyl, alkynyl or acyl moieties optionally have 1 to 6 double bonds or triple bonds.

In some embodiments, at least one of R₁ and R₁′ are not —H.

In some embodiments, m is 0, 1 or 2. In one embodiment, R₂ and R₂′ are H. In another embodiment, the compounds are ethanediol, propanediol or butanediol derivatives of a therapeutic phosphonate. In one embodiment, the compounds of the present invention are ethanedial phosphonate species have the structure:

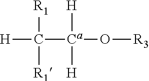

wherein R₁, R₁′, and R₃ are as defined above.

In some embodiments, the compounds of the present invention are propanediol species that have the structure:

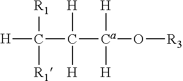

wherein m is 1 and R₁, R₁′, and R₃ are as defined above in the general formula.

In one embodiment, the compounds of the present invention are glycerol species that have the structure:

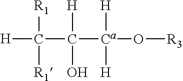

wherein m is 1, R₂ is H, R₂′ is OH, and R₂ and R₂′ on C^{α} are both —H. Glycerol is an optically active molecule. Using the stereospecific numbering convention for glycerol, the sn-3 position is the position which is phosphorylated by glycerol kinase. In compounds of the invention having a glycerol residue, the R₃ moiety may be joined at either the sn-3 or sn-1 position of glycerol.

In some embodiments, R₁ is an alkoxy group having the formula —O—(CH₂)ₜ—CH₃, wherein t is 0-24. In one embodiment, t is 11-19. In another embodiment, t is 15 or 17.

Additionally, antiviral phosphonates such as cidofovir, cyclic-cidofovir, adefovir, tenofovir, and the like, may be used as an R₃ group in accordance with the present invention.

(2) According to one aspect of the present invention, the antiviral compounds have the structure of Formula A, A′ or B

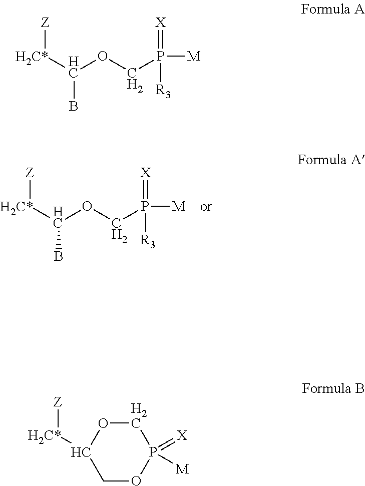

wherein:

M is

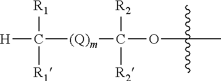

and the O of M is bonded to —P(═X),
wherein R₁, R₁′, R₂ and R₂′ are independently —H, halogen, —ORⁱ, —SRⁱ, —NHRⁱ, —NRⁱRⁱⁱ, and Rⁱ and Rⁱⁱ are independently hydrogen or aliphatic, and

Q, when present, is:

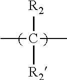

and m is an integer from 0 to 6.

B is selected from the group consisting of hydrogen, F, CF₃, CHF₂, —CH₂CH₃, —CH₂OH, —CH₂CH₂OH, —CH(OH)CH₃, —CH₂F, —CH═CH₂, and —CH₂N₃;

X is selenium, sulphur, or oxygen (in some embodiments, oxygen);

R₃ is hydroxy, —OR₂ₐ, —BH₃, C₁-₈ alkyl, C₂-₈ alkenyl, C₂-₈ alkynyl, C₁-₈ heteroalkyl, C₂-₈ heteroalkenyl, C₂-₈ heteroalkynyl, or —NR′H (in some embodiments, R₂ is hydroxyl);
R₂ₐ is C₁-₈ alkyl, C₂-₈ alkenyl, C₂-₈ alkynyl, C₁-₈ heteroalkyl, C₂-₈ heteroalkenyl, C₂-₈ heteroalkynyl, —P(═O)(OH)₂, or —P(═O)(OH)OP(═O)(OH)₂,
R′ is C₁-₈ alkyl, C₂-₈ alkenyl, C₂-₈ alkynyl, C₁-₈ heteroalkyl, C₂-₈ heteroalkynyl, C₂-₈ heteroalkenyl, C₆-₁₀ aryl, or a substituted or unsubstituted amino acid residue,

Z comprising a heterocyclic moiety comprising at least one N (in some embodiments, the heterocyclic moiety is selected from purine or pyrimidine), and

the symbol * indicates the point of attachment of the methylene moiety in Formula A, A′ or B to Z is via an available nitrogen of the heterocyclic moiety,

or a pharmaceutically acceptable salt thereof.

In some embodiments, the antiviral compound is in the form of an enantiomer, diastereomer, racemate, stereoisomer, tautomer, rotamer or a mixture thereof.

In another embodiment, B is —CH₃ or —CH₂OH.

In some embodiments, R₃ is hydroxyl.

In some embodiments, M is selected from —O—(CH₂)₂—O—C₁₋₂₄alkyl, —O—(CH₂)₃—O—C₁₋₂₄ alkyl, —O—CH₂—CH(OH)—CH₂—O—C₁₋₂₄alkyl, or —O—CH₂—CH(OH)—S—C₁₋₂₄alkyl. In another embodiment, M is —O—(CH₂)ₐ—O—(CH₂)ₜ—CH₃, wherein a is 2 to 4 and t is 11 to 19. In some embodiments, a is 2 or 3 and t is 15 or 17. In some embodiments, M is —O—(CH₂)₂—O—(CH₂)₁₅CH₃ or —O—(CH₂)₂—O—(CH₂)₁₇CH₃. In one embodiment, M is —O—(CH₂)₃—O—(CH₂)₁₅CH₃ or —O—(CH₂)₃—O—(CH₂)₁₇CH₃.

In one embodiment, M is selected from formula a, b or c.

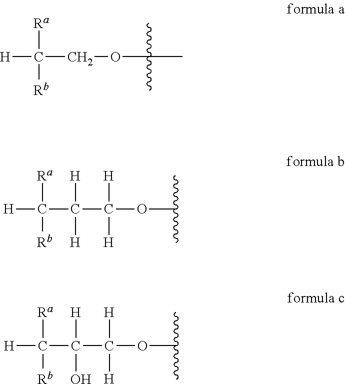

wherein R^{a} and R^{b} are independently —H, halogen, —ORⁱ, —NHRⁱ, —NRⁱRⁱⁱ, and Rⁱ and Rⁱⁱ are independently hydrogen or aliphatic. In some embodiments, Rⁱ and Rⁱⁱ are independently —(C₁-C₂₄)alkyl, —(C₂-C₂₄)alkenyl, —(C₂-C₂₄)alkynyl or —(C₁-C₂₄)acyl.

In some embodiments, at least one of R^{a} or R^{b} is not hydrogen. In some embodiments, R^{a} and R^{b} are independently selected from the group consisting of —H, optionally substituted —O(C₁-C₂₄)alkyl, —O(C₂-C₂₄)alkenyl, —O(C₁-C₂₄)acyl, —S(C₂-C₂₄)alkenyl, and —S(C₁-C₂₄)acyl.

In some embodiments, for M, R₁, R₁′, R₂, or R₂′ is independently —O(C₁-C₂₄)alkyl, —O(C₂-C₂₄)alkenyl, —O(C₂-C₂₄)alkynyl, —O(C₁-C₂₄)acyl, —S(C₁-C₂₄)alkyl, —S(C₂-C₂₄)alkenyl, —S(C₂-C₂₄)alkynyl, —S(C₄₁-C₂₄)acyl, —NH(C₁-C₂₄)alkyl, —NH(C₂-C₂₄)alkenyl, —NH(C₂-C₂₄)alkynyl, —NH(C₁-C₂₄)acyl, —N((C₁-C₂₄)alkyl)((C₂-C₂₄)alkyl), —N((C₁-C₂₄)alkyl)((C₂-C₂₄)alkenyl), —N((C₁-C₂₄)alkyl)((C₂-C₂₄)acyl), —N((C₁-C₂₄)alkyl)((C₂-C₂₄)alkynyl), —N((C₁-C₂₄)alkeyl)((C₂-C₂₄)alkynyl), —N((C₁-C₂₄)alkenyl)((C₂-C₂₄)alkenyl), —N((C₁-C₂₄)alkynyl)((C₂-C₂₄)alkynyl), —N((C₁-C₂₄)acyl)((C₂-C₂₄)alkynyl) or —N((C₁-C₂₄)acyl)((C₂-C₂₄)alkenyl).

In one embodiment, Z comprises purine or pyrimidine, which may be optionally substituted by at least one substituent. In some embodiments, at least one substituent may be selected from the group consisting of halogen, hydroxyl, amino, substituted amino, di-substituted amino, sulfur, nitro, cyano, acetyl, acyl, aza, C₁-₆ alkyl, C₂-₆ alkenyl, C₂-₆ alkynyl, C₆-₁₀ aryl, and carbonyl substituted with a C₁-₆ alkyl, C₂-₆ alkenyl, C₂-₆ alkynyl, or C₆-₁₀ aryl, haloalkyl and aminoalkyl.

In some embodiments, Z may be selected from adenine, 6-chloropurine, xanthine, hypoxanthine, guanine, 8-bromoguanine, 8-chloroguanine, 8-aminoguanine, 8-hydrazinoguanine, 8-hydroxyguanine, 8-methylguanine, 8-thioguanine, 2-aminopurine, 2,6-diaminopurine, thymine, cytosine, 5-fluorocytosine, uracil; 5-bromouracil, 5-iodouracil, 5-ethyluracil, 5-ethynyluracil, 5-propynyluracil, 5-propyluracil, 5-vinyluracil, or 5-bromovinyluracil. In some embodiments, Z is selected from guanin-9-yl, adenin-9-yl, 2,6-diaminopurin-9-yl, 2-aminopurin-9-yl or their 1-deaza, 3-deaza, 8-aza compounds, or cytosin-1-yl. In some embodiments, Z is guanin-9-yl or 2,6-diaminopurin-9-yl.

In another embodiment, Z is selected from 6-alkylpurine and N⁶-alkylpurines, N⁶-acylpurines, N⁶-benzylpurine, 6-halopurine, N⁶-acetylenic purine, N⁶-acyl purine, N⁶-hydroxyalkyl purine, 6-thioalkyl purine, N²-alkylpurines, N⁴-alkylpyrimidines, N⁴-acylpyrimidines, 4-halopyrimidines, N⁴-acetylenic pyrimidines, 4-amino and N⁴-acyl pyrimidines, 4-hydroxyalkyl pyrimidines, 4-thioalkyl pyrimidines, thymine, cytosine, 6-azapyrimidine, including 6-azacytosine, 2- and/or 4-mereaptopyrimidine, uracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-nitropyrimidine, C⁵-aminopyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolopyrimidinyl. Functional oxygen and nitrogen groups on the base can be protected as necessary or desired. Suitable protecting groups are well known to those skilled in the art, and include trimethylsilyl, dimethylhexylsilyl, t-butyldimethylsilyl, and t-butyldiphenylsilyl, trityl, alkyl groups, acyl groups such as acetyl and propionyl, methanesulfonyl, and p-toluenesulfonyl. Preferred bases include cytosine, 5-fluorocytosine, uracil, thymine, adenine, guanine, xanthine, 2,6-diaminopurine, 6-aminopurine, 6-chloropurine and 2,6-dichloropurine.

In one embodiment, Z is

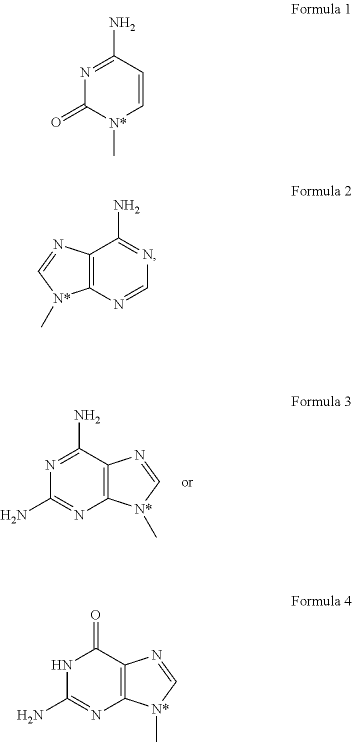

wherein the symbol * in Formula 1-4 indicates the point of attachment of N to the methylene in Formula A, A′ or B.

The example of Z is further described in U.S. Pat. No. 6,583,149, which is incorporated by reference in its entirety.

Additional examples of Z include, but are not limited to, moieties of the general formula:

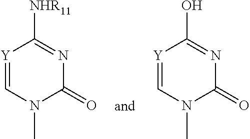

where:

Y is N or CX;

X is selected from the group consisting of H, halo, C₁-₆ alkyl, C₂-₆ alkenyl, C₂-₆ alkynyl, CN, CF₃, N₃, NO₂, C₆-₁₀ aryl, C₆-₁₀ heteroaryl, and COR_{b};
R_{b} is selected from the group consisting of H, OH, SH, C₁-₆ alkyl, C₁-₆ aminoalkyl, C₁-₆ alkoxy and C₁-₆ thioalkyl; and

R₁₁ is selected from the group consisting of H, C₁-₆ alkyl, C₂-₆ alkenyl, C₂-₆ alkynyl, C₃₋₆ cycloalkyl, C₄₋₁₂ alkylcycloalkyl, C₆-₁₀ aryl, and carbonyl substituted with a C₁-₆ alkyl, C₂-₆ alkenyl, C₂-₆ alkynyl, or C₆-₁₀ aryl.

Additional examples of Z include, but are not limited to, compounds of the general formula:

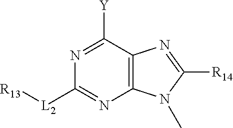

where:

Y is —NRₐR_{b}, or —ORₐ,

L₂ is a covalent bond (that is, is absent), —N(—R₁₅)—, N(—R₁₅)C(═O)—, —O—, —S—, —S(═O)—, or is —S(═O)₂—,

R₁₃ is H, C₁-₆ alkyl, C₁-₆ heteroalkyl, C₂-₆ alkenyl, C₆-₁₀ aryl, C₇-₁₆ arylalkyl, C₃-₁₀ carbocyclyl, C₆-₁₀ heterocyclyl, or C₇-₁₆ heterocyclylalkyl;

R₁₄ is H, halo, hydroxy, alkoxy, —O(CH₂)ₓOC(═O)OR₁₅, or OC(═O)OR₁₅, wherein x is 2 or 3 to 10, 15 or 20,

R₁₅ is H, C₁-₆ alkyl, C₁-₆ heteroalkyl, C₂-₆ alkenyl, C₆-₁₀ aryl, C₇-₁₆ arylalkyl, C₃-₁₀ cycloalkyl, C₆₋₁₀ heterocyclyl, or C₇-₁₆ heterocyclalkyl, and

Rₐ, R_{b} are independently selected from the group consisting of hydrogen, C₁₋₆ alkyl, or C₃₋₆ cycloalkyl, and C₃₋₈ heterocyclyl, wherein C₃₋₆ cycloalkyl and C₃₋₈ heterocyclyl may be optionally substituted with one or more C₁₋₅ alkyl.

Additional examples of Z include, but are not limited to, a moiety of the general formula:

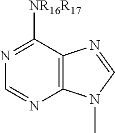

R₁₆ and R₁₇ are independently selected from the group consisting of hydrogen, C₁₋₆ alkyl, or C₃₋₆ cycloalkyl, or C₃₋₈ heterocyclyl, wherein C₃₋₆ cycloalkyl and C₃₋₈ heterocyclyl can be optionally substituted with one or more C₁₋₅ alkyl.

The exemplary compounds of the present invention include, but are not limited to,

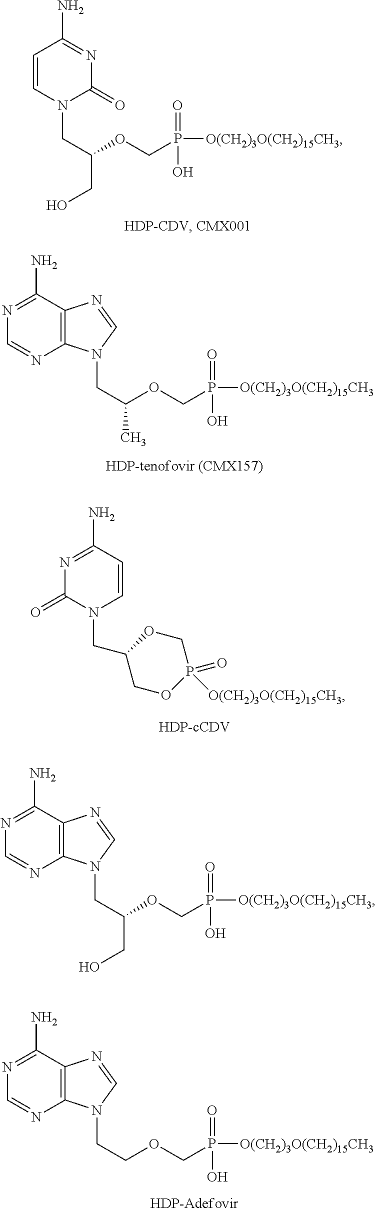

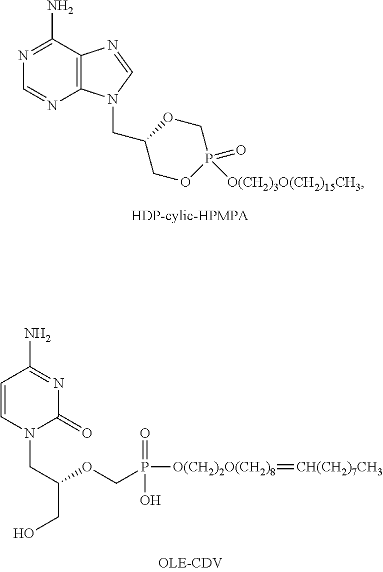

or a pharmaceutically acceptable salt thereof.

More exemplary compounds are shown below:

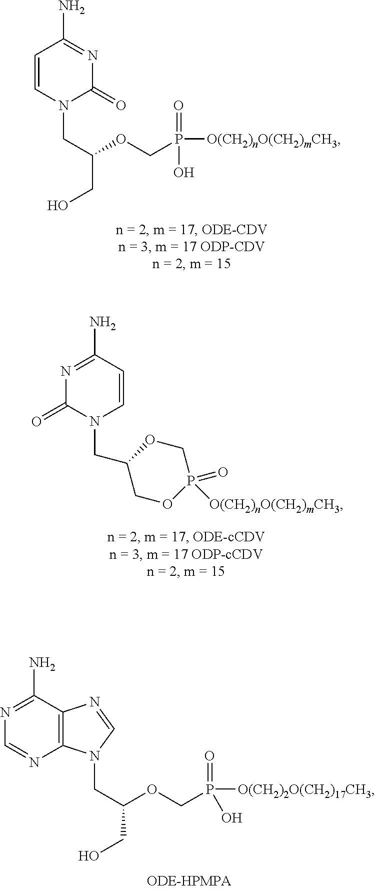

According to a further aspect of the present invention, a variety of lipid derivatives of acyclic nucleotide phosphonates such as cidofovir, tenofovir, cyclic-cidofovir and adefovir can also be used as active agents in the methods and compositions provided herein. In one embodiment, the active agents have the following structures:

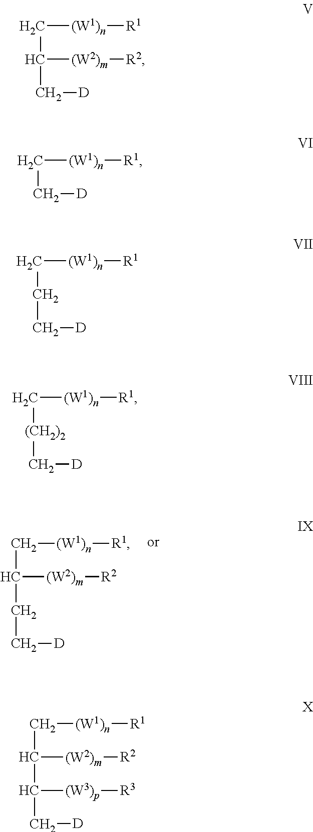

wherein W¹, W², and W³ are each independently —O—, —S—, —SO—, —SO₂, —O(C═O)—, —(C═O)O—, —NH(C═O)—, —(C═O)NH— or —NH—; and in one embodiment are each independently O, S, or —O(C═O)—;

n is 0 or 1; m is 0 or 1; p is 0 or 1;

R¹ is an optionally substituted alkyl, alkenyl or allynyl, e.g., C₁₋₃₀ alkyl, C₂₋₃₀ alkenyl, or C₂₋₃₀ alkynyl; or in one embodiment, R¹ is optionally substituted C₈₋₃₀ alkyl, C₈₋₃₀ alkenyl or C₈₋₃₀ alkynyl, or R¹ is a C_{g-24} alkyl, C₈₋₂₄ alkenyl or C₈₋₂₄ alkynyl (e.g., C₁₇, C₁₈, C₁₉, C₂₀, C₂₁, C₂₂, C₂₃, or C₂₄ alkyl, alkenyl, or alkynyl);

R² and R³ are each independently an optionally substituted C₁₋₂₅ alkyl, C₂₋₂₅ alkenyl, or C₂₋₂₅ alkynyl;

D may be cidofovir, tenofovir, cyclic cidofovir or adefovir directly linked to a methylene group as depicted in Formulas V-X. For example, when D is tenofovir, D is a moiety of the formula:

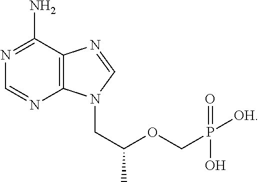

When D is cidofovir, D is a moiety of the formula:

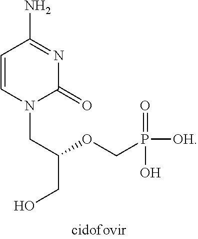

(e.g., Cidofovir or tenofovir is directly linked to the methylene group of formula V-X via the phosphonate hydroxyl group).

In some embodiments of formulas V-X:
W¹, W², and W³ are each independently —O—, —S—, or —O(CO)—;
n is 0 or 1; m is 0 or 1; p is 0 or 1;
R¹ is optionally substituted C₁₂₋₂₄ alkyl or alkenyl (e.g., C₁₂, C₁₃, C₁₄, C₁₅, C₁₆, C₁₇, C₁₈, C₁₉, C₂₀, C₂₁, C₂₂, C₂₃, or C₂₄ alkyl or alkenyl);
R² and R³ are each independently optionally substituted C₁₋₂₄ alkyl or C₂₋₂₄ alkenyl, or C₂₋₂₄ alkynyl.
D is cidofovir, tenofovir, cyclic cidofovir or adefovir linked directly to a methylene group as depicted in Formulas V-X.

In another embodiment, the active compound of Formula V-X has one of the following structures: wherein R¹ is an optionally substituted C₈₋₂₄ alkyl, for example, C₁₂₋₂₄ alkyl, D is cidofovir, tenofovir, cyclic cidofovir or adefovir linked directly to a methylene group as depicted in Formulas V-X.

(3) Compounds, compositions, formulations, and methods of treating subjects that can be used to carry out the present invention include, but are not limited to, those described in U.S. Pat. Nos. 6,716,825, 7,034,014, 7,094,772, 7,098,197, 7,452,898, and 7,687,480, the disclosures of which are incorporated by reference herein in their entireties.

In some embodiments, the active compounds have the structure Formula C:

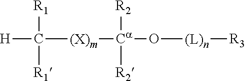

### Formula C

wherein:

R₁, R₁′, R₂ and R₂′ are independently —H, halogen, —NH₂, —OH, or —SH or optionally substituted —XRⁱ, and wherein X is O, S, —NH, or —NRⁱⁱ, and Rⁱ and Rⁱⁱ are independently —(C₁-C₂₄)alkyl, —(C₁-C₂₄)alkenyl, —(C₁-C₂₄)alkynyl, or —(C₁-C₂₄)acyl.

In some embodiments, at least one of R₁ and R₁′ are not —H. In some embodiments, said alkenyl or acyl moieties optionally have 1 to 6 double bonds,

R₃ is a pharmaceutically active phosphonate, bisphosphonate or a phosphonate derivative of a pharmacologically active compound, linked to a functional group on optional linker L or to an available oxygen atom on C^{α};

X, when present, is:

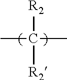

L is a valence bond or a bifunctional linking molecule of the formula -J-(CR₂)ₜ-G-, wherein t is an integer from 1 to 24, J and G are independently —O—, —S—, —C(O)O—, or —NH—, and R is —H, substituted or unsubstituted alkyl, or alkenyl;

m is an integer from 0 to 6; and

n is 0 or 1.

In some embodiments, m=0, 1 or 2. In some embodiments, R₂ and R₂′ are H, and the prodrugs are then ethanediol, propanediol or butanediol derivatives of a therapeutic phosphonate. A exemplary ethanediol phosphonate species has the structure:

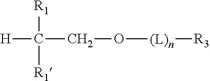

wherein R₁, R₁′, R₃, L, and n are as defined above.

In some embodiments, propanediol species has the structure:

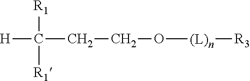

wherein m=1 and R1, R1′, R3, L and n are as defined above in the general formula.

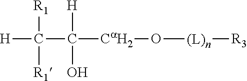

wherein m=1, R₂═H, R₂′═OH, and R₂ and R₂′ on C^{α} are both —H. Glycerol is an optically active molecule. Using the stereospecific numbering convention for glycerol, the sn-3 position is the position which is phosphorylated by glycerol kinase. In compounds of the invention having a glycerol residue, the -(L)ₙ-R₃ moiety may be joined at either the sn-3 or sn-1 position of glycerol.

In another embodiment, R₁ is an alkoxy group having the formula —O—(CH₂)ₜ—CH₃, wherein t is 0-24. More preferably t is 11-19. Most preferably t is 15 or 17.

Exemplary R₃ groups include bisphosphonates that are known to be clinically useful, for example, the compounds:

Etidronate: 1-hydroxyethylidene bisphosphonic acid (EDHP);

Clodronate: dichloromethylene bisphosphonic acid (Cl₂ MDP);

Tiludromate: chloro-4-phenylthiomethylene bisphosphonic acid;

Pamidronate: 3-amino-1-hydroxypropylidene bisphosphonic acid (ADP);

Alendronate: 4-amino-1-hydroxybutylidene bisphosphonic acid;

Olpadronate: 3-dimethylamino-1-hydroxypropylidene bisphosphonic acid (dimethyl-APD);

Ibandronate: 3-methylpentylamino-1-hydroxypropylidene bisphosphonic acid (BM 21.0955);

EB-1053: 3-(1-pyrrolidinyl)-1-hydroxypropylidene bisphosphonic acid;

Risedmnate: 2-(3-pyridinyl)-1-hydroxy-ethylidene bisphosphonic acid;

Amino-Olpadronate: 3-(N,N-diimethylamino-1-aminopropylidene)bisphosphonate (IG9402), and the like.

R₃ may also be selected from a variety of phosphonate-containing nucleotides (or nucleosides which can be derivatized to their corresponding phosphonates), which are also contemplated for use herein. Preferred nucleosides include those useful for treating disorders caused by inappropriate cell proliferation such as 2-chloro-deoxyadenosine, 1-β-D-arabinofuranosyl-cytidine (cytarabine, ara-C), fluorouridine, fluorodeoxyuridine (floxuridine), gemcitabine, cladribine, fludarabine, pentostatin (2′-deoxycoformycin), 6-mercaptopurine, 6-thioguanine, and substituted or unsubstituted arabinofuranosyl-guanine (ara-G), 1-β-D-arabinofuranosyl-adenosine (ara-A), 1-β-D-arabinofuranosyl-uridine (ara-U), and the like.

Nucleosides useful for treating viral infections may also be converted to their corresponding 5′-phosphonates for use as an R₃ group. Such phosphonate analogs typically contain either a phosphonate (—PO₃H₂) or a methylene phosphonate (—CH₂—PO₃H₂) group substituted for the 5′-hydroxyl of an antiviral nucleoside. Some examples of antiviral phosphonates derived by substituting —PO₃H₂ for the 5′-hydroxyl are:

| | | |
|---|---|---|
| 3′-azido-3′,5′- dideoxythymidine-5′- phosphonic acid (AZT phosphonate) | 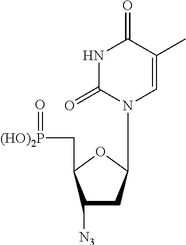 | Hakimelahi, G. H.; Moosavi- Movahedi, A. A.; Sadeghi, M. M.; Tsay, S-C.; Hwu, J. R. J. Med. Chem. 1995, 38: 4648- 4659. |
| 3′,5′-dideoxythymidine-2′-ene- 5′-phosphonic acid (d4T phosphonate) | 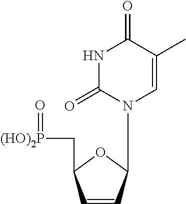 | Hakimelahi, G. H.; Moosavi- Movahedi, A. A.; Sadeghi, M. M.; Tsay, S-C.; Hwu, J. R. J. Med. Chem. 1995, 38: 4648- 4659. |
| 2′,3′,5′-trideoxycytidine-5′- phosphonic acid (ddC phosphonate) | 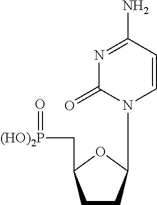 | Kofoed, T., Ismail, A. E. A. A.; Pedersen, E. B.; Nielsen, C. Bull. Soc. Chim. Fr. 1997, 134: 59-65. |
| 9-[3-(phosphono- methoxy)propyl]adenine (Adefovir) | 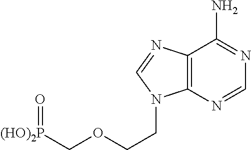 | Kim, C. U.; Luh, B. Y.; Misco, P. F.; Bronson, J. J.; Hitchcock, M. J. M.; Ghazzouli, I.; Martin, J. C. J. Med. Chem. 1990, 33: 1207-1213. |

Some examples of antiviral phosphonates derived by substituting —CH₂—PO₃H₂ for the hydroxyl are:

| | | |
|---|---|---|
| Ganciclovir phosphonate | 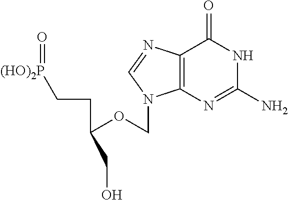 | Huffman, J. H.; Sidwell, R. W.; Morrison, A. G.; Coombs, J., Reist, E. J. Nucleoside Nucleotides, 1994, 13: 607-613. |
| Acyclovir phosphonate | 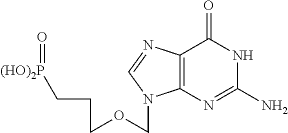 | Huffman, J. H.; Sidwell, R. W.; Morrison, A. G.; Coombs, J., Reist, E. J. Nucleoside Nucleotides, 1994, 13: 607-613. |
| Ganciclovir cyclic phosphonate | 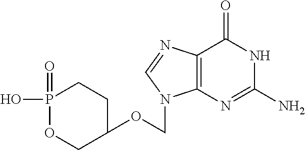 | Smee, D. F.; Reist, E. J. Antimicrob. Agents Chemother. 1996, 40: 1964-1966. |
| 3′-thia-2′,3′- dideoxycytidine-5′- phosphonic acid | 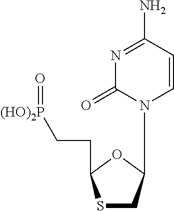 | Kraus, J. L.; Nucleosides Nucleotides, 1993, 12: 157-162. |

Other exemplary antiviral nucleotide phosphonates are derived similarly from antiviral nucleosides including ddA, ddI, ddG, L-FMAU, DXG, DAPD, L-dA, L-dI, L-(d)T, L-dC, L-dG, FTC, penciclovir, and the like.

Additionally, antiviral phosphonates such as cidofovir, cyclic cidofovir, adefovir, tenofovir, and the like, may be used as an R₃ group in accordance with the present invention.

Many phosphonate compounds exist that can be derivatized according to the invention to improve their pharmacologic activity, or to increase their oral absorption, such as, for example, the compounds disclosed in the following patents, each of which are hereby incorporated by reference in their entirety: U.S. Pat. No. 3,468,935 (Etidronate), U.S. Pat. No. 4,327,039 (Pamidronate), U.S. Pat. No. 4,705,651 (Alendronate), U.S. Pat. No. 4,870,063 (Bisphosphonic acid derivatives), U.S. Pat. No. 4,927,814 (Diphosphonates), U.S. Pat. No. 5,043,437 (Phosphonates of azidodideoxynucleosides), U.S. Pat. No. 5,047,533 (Acyclic purine phosphonate nucleotide analogs), U.S. Pat. No. 5,142,051 (N-Phosphonylmethoxyalkyl derivatives of pyrimidine and purine bases), U.S. Pat. No. 5,183,815 (Bone acting agents), U.S. Pat. No. 5,196,409 (Bisphosphonates), U.S. Pat. No. 5,247,085 (Antiviral purine compounds), U.S. Pat. No. 5,300,671 (Gem-diphosphonic acids), U.S. Pat. No. 5,300,687 (Trifluoromethylbenzylphosphonates), U.S. Pat. No. 5,312,954 (Bis- and tetrakis-phosphonates), U.S. Pat. No. 5,395,826 (Guanidinealkyl-1,1-bisphosphonic acid derivatives), U.S. Pat. No. 5,428,181 (Bisphosphonate derivatives), U.S. Pat. No. 5,442,101 (Methylenebisphosphonic acid derivatives), U.S. Pat. No. 5,532,226 (Trifluoromethybenzylphosphonates), U.S. Pat. No. 5,656,745 (Nucleotide analogs), U.S. Pat. No. 5,672,697 (Nucleoside-5′-methylene phosphonates), U.S. Pat. No. 5,717,095 (Nucleotide analogs), U.S. Pat. No. 5,760,013 (Thymidylate analogs), U.S. Pat. No. 5,798,340 (Nucleotide analogs), U.S. Pat. No. 5,840,716 (Phosphonate nucleotide compounds), U.S. Pat. No. 5,856,314 (Thio-substituted, nitrogen-containing, heterocyclic phosphonate compounds), U.S. Pat. No. 5,885,973 (olpadronate), U.S. Pat. No. 5,886,179 (Nucleotide analogs), U.S. Pat. No. 5,877,166 (Enantiomerically pure 2-aminopurine phosphonate nucleotide analogs), U.S. Pat. No. 5,922,695 (Antiviral phosphonomethoxy nucleotide analogs), U.S. Pat. No. 5,922,696 (Ethylenic and allenic phosphonate derivatives of purines), U.S. Pat. No. 5,977,089 (Antiviral phosphonomethoxy nucleotide analogs), U.S. Pat. No. 6,043,230 (Antiviral phosphonomethoxy nucleotide analogs), U.S. Pat. No. 6,069,249 (Antiviral phosphonomethoxy nucleotide analogs); Belgium Patent No. 672205 (Clodronate); European Patent No. 753523 (Amino-substituted bisphosphonic acids); European Patent Application 186405 (geminal diphosphonates); and the like.

Certain bisphosphonate compounds have the ability to inhibit squalene synthase and to reduce serum cholesterol levels in mammals, including man. Examples of these bisphosphonates are disclosed, for example, in U.S. Pat. Nos. 5,441,946 and 5,563,128 to Pauls et al. Phosphonate derivatives of lipophilic amines, both of which are hereby incorporated by reference in their entirety. Analogs of these squalene synthase inhibiting compounds according to the invention, and their use in the treatment of lipid disorders in humans are within the scope of the present invention. Bisphosphonates of the invention may be used orally or topically to prevent or treat periodontal disease as disclosed in U.S. Pat. No. 5,270,365, hereby incorporated by reference in its entirety.

In some embodiments, the active compounds have a phosphonate ester formed by a covalent linking of an antiviral compound selected from the group consisting of cidofovir, adefovir, cyclic cidofovir and tenofovir, to an alcohol selected from the group consisting of an alkylglycerol, alkylpropanediol, 1-S-alkylthioglycerol, alkoxyalkanol or allylethanediol, or a pharmaceutically acceptable salt thereof.

In some embodiments, the active compounds comprise an antiviral nucleoside compound, wherein the 5′-hydroxyl group has been substituted for a phosphonate or methyl phosphonate that is covalently linked to an alkylethanediol.

Certain compounds of the invention possess one or more chiral centers, e.g. in the sugar moieties, and may thus exist in optically active forms. Likewise, when the compounds contain an alkenyl group or an unsaturated alkyl or acyl moiety there exists the possibility of cis- and trans-isomeric forms of the compounds. Additional asymmetric carbon atoms can be present in a substituent group such as an alkyl group. The R- and S-isomers and mixtures thereof, including racemic mixtures as well as mixtures of cis- and trans-isomers are contemplated by this invention. All such isomers as well as mixtures thereof are intended to be included in the invention. If a particular stereoisomer is desired, it can be prepared by methods well known in the art by using stereospecific reactions with starting materials that contain the asymmetric centers and are already resolved or, alternatively, by methods that lead to mixtures of the stereoisomers and resolution by known methods.

In cases where compounds are sufficiently basic or acidic to form stable nontoxic acid or base salts, administration of the compound as a pharmaceutically acceptable salt may be appropriate. Pharmaceutically acceptable salts are salts that retain the desired biological activity of the parent compound and do not impart undesired toxicological effects. Pharmaceutically acceptable salts include those derived from pharmaceutically acceptable inorganic or organic bases and acids. Suitable salts include those derived from alkali metals such as potassium and sodium; alkaline earth metals such as calcium and magnesium; or derive from ammonium, ammonium salts with organic bases such as dicyclohexylamine and N-methyl-D-glucamine, among numerous other acids well known in the pharmaceutical art. In some embodiments, the pharmaceutically acceptable salts are selected from organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Exemplary agent that may be used to form the salt include, but are not limited to, (1) acid such as inorganic acid, for example, hydrochloric acid, hydrobromic acid, sulfuric acid, phosphoric acid, nitric acid; or organic acid for example, acetic acid, citric acid, fumaric acid, alginic acid, gluconic acid, gentisic acid, hippuric acid, benzoic acid, maleic acid, tannic acid, L-mandelic acid, orotic acid, oxalic acid, saccharin, succinic acid, L-tartaric acid, ascorbic acid, palmitic acid, polyglutamic acid, toluenesulfonic acid, naphthalenesulfonic acid, methanesulfonic acid, naphthalenedisulfonic acid, polygalacturonic acid, (2) bases such as ammonia, mono or di-substituted ammonia, alkali metal bases such as potassium hydroxide, lithium hydroxide, sodium hydroxide; alkaline earth bases such as magnesium hydroxide, calcium hydroxide; organic bases such as larginine, diethylamine, diethylaminoethanol, dicyclohexylamine, ethylenediamine, imidazole, L-lysine, 2-hydroxyethylmorpholine, N-methyl-glucamine, potassium methanolate, zinc tert-butoxide.

Active compounds as described herein can be prepared in accordance with known procedures, or variations thereof that will be apparent to those skilled in the art. See, e.g., Painter et al., Evaluation of Hexadecyloxypropyl-9-R-[2-(Phosphonomethoxy)Propyl]-Adenine, CMX157, as a Potential Treatment for Human Immunodeficiency Virus Type 1 and Hepatitis B Virus Infections, Antimicrobial Agents and Chemotherapy 51, 3505-3509 (2007) and US Patent Application Publication No. 200710003516 to Almond et al.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### B. Synthesis of Active Compounds

The process to be utilized in the preparation of the compounds described herein depends upon the specific compound desired. Such factors as the selection of the specific substituent and various possible locations of the specific substituent all play a role in the path to be followed in the preparation of the specific compounds of this invention. Those factors are readily recognized by one of ordinary skill in the art.

In general, the compounds of this invention may be prepared by standard techniques known in the art and by known processes analogous thereto. General methods for preparing compounds of the present invention are set forth below.

In the following description, all variables are, unless otherwise noted, as defined in the formulas described herein. The following non-limiting descriptions illustrate the general methodologies that may be used to obtain the compounds described herein.

Compounds (or “prodrugs”) useful in the invention can be prepared in a variety of ways, as generally depicted in Schemes I-VI of U.S. Pat. No. 6,716,825. The general phosphonate esterification methods described below are provided for illustrative purposes only and are not to be construed as limiting this invention in any manner. Indeed, several methods have been developed for direct condensation of phosphonic acids with alcohols (see, for example, R. C. Larock, Comprehensive Organic Transformations, VCH, New York, 1989, p. 966 and references cited therein). Isolation and purification of the compounds and intermediates described in the examples can be effected, if desired, by any suitable separation or purification procedure such as, for example, filtration, extraction, crystallization, flash column chromatography, thin-layer chromatography, distillation or a combination of these procedures. Specific illustrations of suitable separation and isolation procedures are in the examples below. Other equivalent separation and isolation procedures can of course, also be used.

Scheme I of U.S. Pat. No. 6,716,825 outlines a synthesis of bisphosphonate prodrugs that contain a primary amino group, such as pamidronate or alendronate. Example 1 therein provides conditions for a synthesis of 1-O-hexadecyloxypropyl-alendronate (HDP-alendronate) or 1-O-hexadecyloxypropyl-pamidronate (HDP-pamidronate). In this process, a mixture of dimethyl 4-phthalimidobutanoyl phosphonate (1b, prepared as described in U.S. Pat. No. 5,039,819)) and hexadecyloxypropyl methyl phosphite (2) in pyridine solution is treated with triethylamine to yield bisphosphonate tetraester 3b which is purified by silica gel chromatography. Intermediate 2 is obtained by transesterification of diphenyl phosphite as described in Kers, A., Kers, I., Stawinski, J., Sobkowski, M., Kraszewski, A. Synthesis, April 1995, 427 430. Thus, diphenyl phosphite in pyridine solution is first treated with hexadecyloxypropan-1-ol, then with methanol to provide compound 2.

An important aspect of the process is that other long chain alcohols may be used in place of hexadecyloxypropan-1-ol to generate the various compounds of this invention. Treatment of intermediate 3b with bromotrimethylsilane in acetonitrile cleaves the methyl esters selectively to yield monoester 4b. Treatment of 4b with hydrazine in a mixed solvent system (20% methanol/80% 1, 4-dioxane) results in removal of the phthalimido protecting group as shown. The desired alendronate prodrug is collected by filtration and converted to the triammonium salt by treatment with methanolic ammonia.

Scheme II of U.S. Pat. No. 6,716,825 illustrates a synthesis of analogs of bisphosphonates lacking a primary amino group, in this case the process steps are similar to those of Scheme 1 except that protection with a phthalimido group and subsequent deprotection by hydrazinolysis are unnecessary. Bisphosphonates having 1-amino groups, such as amino-olpadronate, maybe converted to analogs according to the invention prodrugs using a slightly modified process shown in Scheme III of U.S. Pat. No. 6,716,825. Treatment of a mixture of compound 2 and 3-(dimethylamino)propionitrile with dry HCl followed by addition of dimethyl phosphite affords tetraester 3 which, after demethylation with bromotrimethylsilane, yields hexadecyloxypropyl-amino-olpadronate.

Scheme IV of U.S. Pat. No. 6,716,825 illustrates synthesis of a bisphosphonate analog where the lipid group is attached to a primary amino group of the parent compound rather than as a phosphonate ester.

Scheme V of U.S. Pat. No. 6,716,825 illustrates a general synthesis of alkylglycerol or alkylpropanediol analogs of cidofovir, cyclic cidofovir, and other phosphonates. Treatment of 2, 3-isopropylidene glycerol, 1, with NaH in dimethylformamide followed by reaction with an alkyl methanesulfonate yields the alkyl ether, 2. Removal of the isopropylidene group by treatment with acetic acid followed by reaction with trityl chloride in pyridine yields the intermediate 3. Alkylation of intermediate 3 with an alkyl halide results in compound 4. Removal of the trityl group with 80% aqueous acetic acid affords the O,O-dialkyl glycerol, 5. Bromination of compound 5 followed by reaction with the sodium salt of cyclic cidofovir or other phosphonate-containing nucleotide yields the desired phosphonate adduct, 7. Ring-opening of the cyclic adduct is accomplished by reaction with aqueous sodium hydroxide. The compound of propanediol species may be synthesized by substituting 1-O-alkylpropane-3-ol for compound 5 in Scheme V. The tenofovir and adefovir analogs may be synthesized by substituting these nucleotide phosphonates for cCDV in reaction (f) of Scheme V. Similarly, other nucleotide phosphonates of the invention may be formed in this manner.

Scheme VI of U.S. Pat. No. 6,716,825 illustrates a general method for the synthesis of nucleotide phosphonates of the invention using 1-O-hexadecyloxypropyl-adefovir as the example. The nucleotide phosphonate (5 mmol) is suspended in dry pyridine and an alkoxyalkanol or alkylglycerol derivative (6 mmol) and 1,3-dicyclohexylcarbodiimde (DCC, 10 mmol) are added. The mixture is heated to reflux and stirred vigorously until the condensation reaction is complete as monitored by thin-layer chromatography. The mixture is then cooled and filtered. The filtrate is concentrated under reduced pressure and the residues adsorbed on silica gel and purified by flash column chromatography (elution with approx. 9:1 dichloromethane/methanol) to yield the corresponding phosphonate monoester.

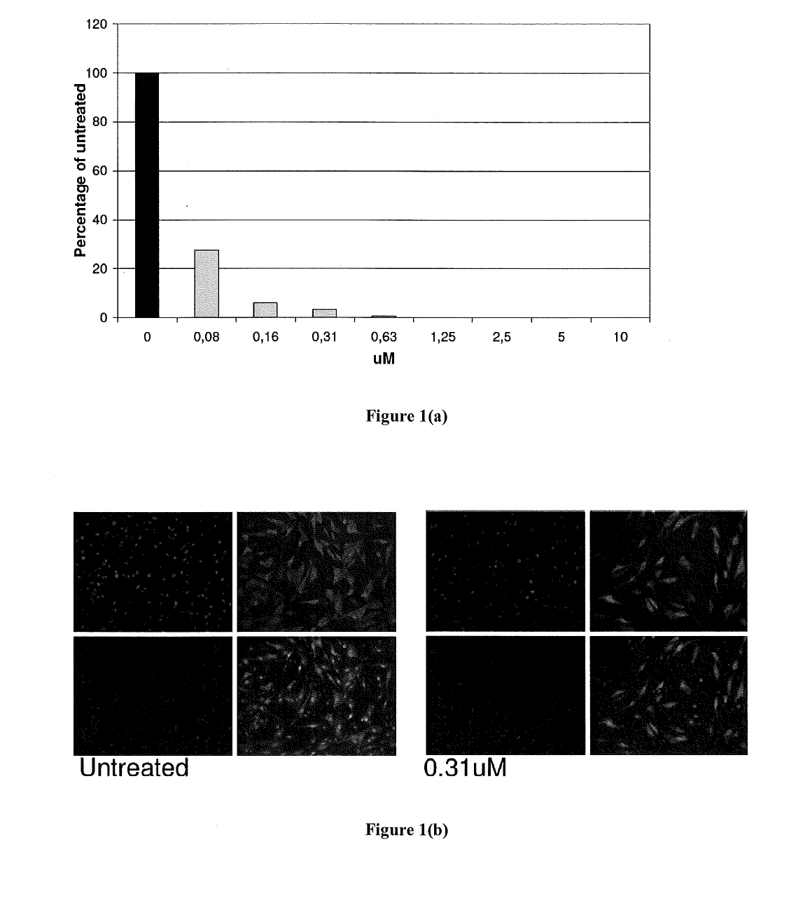

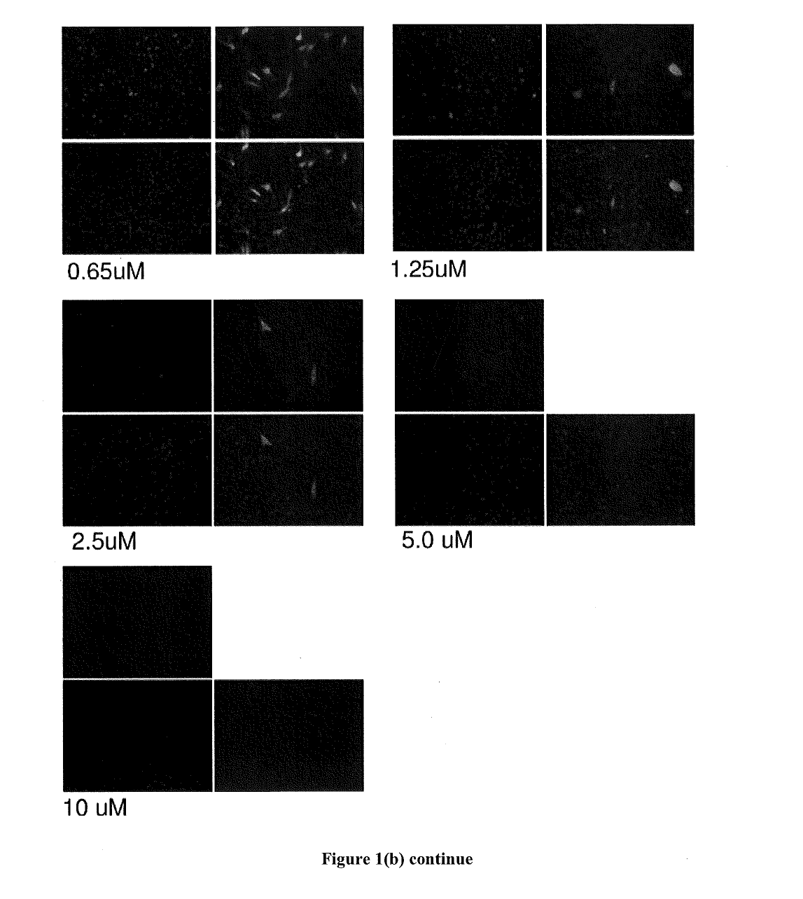

Scheme VII (which is referenced as FIG. 1 in Kern et al., AAC 46 (4):991) illustrates the synthesis for alkoxyalkyl analogs of cidofovir (CDV) and cyclic cidofovir (cCDV). In FIG. 1, the arrows indicate the following reagents: (a) N,N-dicyclohexylmorpholinocarboxamide, N,N-dicyclohexylcarbodiimide, pyridine, 100° C.; (b) 1-bromo-3-octadecyloxyethane (ODE), or 1-bromo-3-hexadecyloxypropane (HDP), N,N-dimethylformamide, 80° C.; (c) 0.5 M NaOH.

One skilled in the art should be able to convert the salt disclosed in the application to free acid by using any applicable methods known to one skilled in the art.

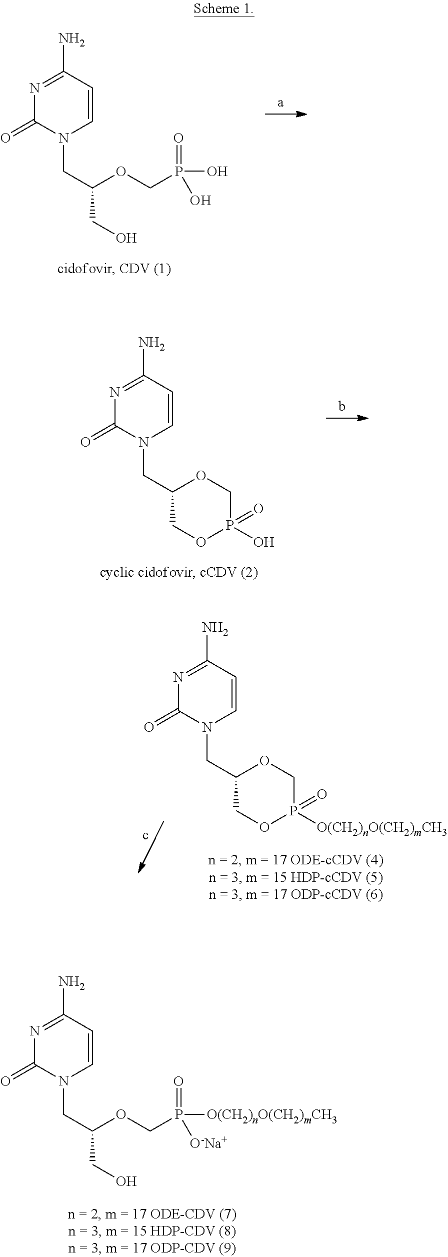

As described herein, compounds of the invention may optionally be substituted with one or more substituents, such as are illustrated generally above, or as exemplified by particular classes, subclasses, and species of the invention. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention may be those that result in the formation of stable or chemically feasible compounds.

### C. Pharmaceutical Formulations and Administration

In one embodiment, the present invention is a pharmaceutical composition comprising the compounds described herein. In another embodiment, the pharmaceutical composition further comprises a pharmaceutically acceptable carrier. The term “pharmaceutically acceptable carrier” as used herein refers to any substance, not itself a therapeutic agent, used as a vehicle for delivery of a therapeutic agent to a subject. Examples of pharmaceutically acceptable carriers and methods of manufacture for various compositions include, but are not limited to, those described in Remington's Pharmaceutical Sciences, 18th Ed., Mack Publishing Co. (1990) (See also US Patent Application US 2007/0072831).

In some embodiments, the pharmaceutical composition further comprises one or more immunosuppressive agents described in Section E.

While it is possible for the active ingredients to be administered alone it is preferably to present them as pharmaceutical formulations. The formulations, both for veterinary and for human use, of the present invention comprise at least one active ingredient, as above defined, together with one or more pharmaceutically acceptable carriers (excipients, diluents, etc.) thereof and optionally other therapeutic ingredients. The carrier(s) must be “acceptable” in the sense of being compatible with the other ingredients of the formulation and not deleterious to the recipient thereof.

The compounds of the invention may be formulated with conventional carriers, diluents and excipients, which will be selected in accord with ordinary practice. Tablets will contain excipients, glidants, fillers, binders, diluents and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Any suitable route of administration may be employed for providing a mammal, especially a human with an effective dosage of a compound of the present invention. For example, the compositions of the present invention may be suitable for formulation for oral, parenteral (including subcutaneous, intramuscular, intravenous, intradermal, intrathecal and epidural), inhalation spray, topical, rectal, nasal, sublingual, buccal, vaginal or implanted reservoir administration, etc. In some embodiments, the compositions are administered orally, topically, intraperitoneally or intravenously. These suspensions may be formulated according to techniques known in the art using suitable dispersing or wetting agents and suspending agents. Among the acceptable vehicles and solvents that may be employed are water, Ringer's solution and isotonic sodium chloride solution. In addition, sterile, fixed oils are conventionally employed as a solvent or suspending medium.

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

A pharmaceutically acceptable oil may be employed as a solvent or suspending medium in compositions of the present invention. Fatty acids, such as oleic acid and its glyceride derivatives are suitably included in injectable formulations, as are natural pharmaceutically acceptable oils, such as olive oil or castor oil, especially in their polyoxyethylated versions. The oil containing compositions of the present invention may also contain a long-chain alcohol diluent or dispersant, such as carboxymethyl cellulose or similar dispersing agents that are commonly used in the formulation of pharmaceutically acceptable dosage forms including emulsions and suspensions. The compositions suitably further comprise surfactants (such as non-ionic detergents including Tween® or Span®) other emulsifying agents, or bioavailability enhancers.

The compositions of this invention may be in the form of an orally acceptable dosage form including, but not limited to, capsules, tablets, suspensions or solutions. The oral dosage form may include at least one excipient. Excipients used in oral formulations of the present can include diluents, substances added to mask or counteract a disagreeable taste or odor, flavors, dyes, fragrances, and substances added to improve the appearance of the composition. Some oral dosage forms of the present invention suitably include excipients, such as disintegrants, binding agents, adhesives, wetting agents, polymers, lubricants, or glidants that permit or facilitate formation of a dose unit of the composition into a discrete article such as a capsule or tablet suitable for oral administration. Excipient-containing tablet compositions of the invention can be prepared by any suitable method of pharmacy which includes the step of bringing into association one or more excipients with a compound of the present invention in a combination of dissolved, suspended, nanoparticulate, microparticulate or controlled-release, slow-release, programmed-release, timed-release, pulse-release, sustained-release or extended-release forms thereof.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents such as pentamidine for treatment of pneumocystis pneumonia.

Formulations suitable for vaginal administration may be presented as pessaries, rings, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Pharmaceutically acceptable compositions of the present invention may be in the form of a topical solution, ointment, or cream in which the active component is suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Where the topical formulation is in the form of an ointment or cream, suitable carriers include, but are not limited to, mineral oil, sorbitan monostearate, polysorbate 60, cetyl esters wax, cetearyl alcohol, 2 octyldodecanol, benzyl alcohol and water. In some embodiments, the topical composition of the present invention is in the form of a spray.

The pharmaceutically acceptable compositions of this invention may also be administered by nasal, aerosol or by inhalation administration routes. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. In some embodiments, the nasal administration of the composition of the present invention is in the form of a spray. Any suitable carrier for spray application may be used in the present invention.

Alternatively, pharmaceutically acceptable compositions of this invention may be in the form of a suppository for rectal administration. The suppositories can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Additionally, the pharmaceutical formulation including compounds of the present invention can be in the form of a parenteral formulation. The term “parenteral” as used herein includes subcutaneous, intravenous, intramuscular, intra-articular, intra-synovial, intrasternal, intrathecal, intrahepatic, intralesional and intracranial injection or infusion techniques.

In certain embodiments, the pharmaceutically compositions of this invention are formulated for oral administration. For oral administration to humans, the dosage range is 0.01 to 1000 mg/kg body weight in divided doses. In one embodiment the dosage range is 0.1 to 100 mg/kg body weight in divided doses. In another embodiment the dosage range is 0.5 to 20 mg/kg body weight in divided doses. For oral administration, the compositions may be provided in the form of tablets or capsules containing 1.0 to 1000 milligrams of the active ingredient, particularly, 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated.

It should also be understood that a specific dosage and treatment regimen for any particular patient will depend upon a variety of factors, including the activity of the specific compound employed, the mode of administration, the age, body weight, general health, gender, diet, rate of excretion, drug combination, and the judgment of the treating physician, the condition being treated and the severity of the condition. Such dosage may be ascertained readily by a person skilled in the art. This dosage regimen may be adjusted to provide the optimal therapeutic response.

The present invention further provides veterinary compositions comprising at least one active ingredient as above defined together with a veterinary carrier Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can be used to provide controlled release pharmaceutical formulations containing as active ingredient one or more compounds of the invention (“controlled release formulations”) in which the release of the active ingredient can be controlled and regulated to allow less frequent dosing or to improve the pharmacokinetic or toxicity profile of a given invention compound. Controlled release formulations adapted for oral administration in which discrete units comprising one or more compounds of the invention can be prepared according to conventional methods. Controlled release formulations may be employed for the treatment or prophylaxis of various viral infections and/or viral induced tumor. Exemplary viral infection and/or viral induced tumor include, but are not limited to, human viral infections or tumor associated with virus such as Herpes viruses (CMV, HSV 1, HSV 2, VZV, and the like), papillomavirus, polyomavirus (JC virus, BK virus, SV 40, etc), retroviruses, adenoviruses and the like. The controlled release formulations can also be used to treat HIV infections and related conditions such as tuberculosis, malaria, pneumocystis pneumonia, CMV retinitis, AIDS, AIDS-related complex (ARC) and progressive generalized lymphadeopathy (PGL), and AIDS-related neurological conditions such as multiple sclerosis, and tropical spastic paraparesis. Other human retroviral infections that may be treated with the controlled release formulations according to the invention include Human T-cell Lymphotropic virus and HIV-2 infections. The invention accordingly provides pharmaceutical formulations for use in the treatment or prophylaxis of the above-mentioned human or veterinary conditions.

Pharmacokinetic Enhancers.

The compounds of the invention may be employed in combination with pharmacokinetic enhancers (sometimes also referred to as “booster agents”). One aspect of the invention provides the use of an effective amount of an enhancer to enhance or “boost” the pharmacokinetics of a compound of the invention. An effective amount of an enhancer, for example, the amount required to enhance an active compound or additional active compound of the invention, is the amount necessary to improve the pharmacokinetic profile or activity of the compound when compared to its profile when used alone. The compound possesses a better efficacious pharmacokinetic profile than it would without the addition of the enhancer. The amount of pharmacokinetic enhancer used to enhance the potency of the compound is, preferably, subtherapeutic (e.g., dosages below the amount of booster agent conventionally used for therapeutically treating infection in a patient). An enhancing dose for the compounds of the invention is subtherapeutic for treating infection, yet high enough to effect modulation of the metabolism of the compounds of the invention, such that their exposure in a patient is boosted by increased bioavailability, increased blood levels, increased half life, increased time to peak plasma concentration, increased/faster inhibition of HIV integrase, RT or protease and/or reduced systematic clearance. One example of a pharmacokinetic enhancer is RITONAVIR™ (Abbott Laboratories).

Combinations.

As noted above, the compositions of the present invention can include the active compounds as described in section A above in combination with one or more (e.g., 1, 2, 3) immunosuppressant agents such as described in section E below, in analogous manner as known in the art.

Specific examples of such combinations include, but are not limited to: CMX001 or a pharmaceutically acceptable salt thereof in combination with at least one immunosuppressant agents selected from Daclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm and a combination thereof. In some embodiments, the pharmaceutical composition includes CMX001, Tysabri (natalizumab), and a pharmaceutically acceptable carrier.

### D. Methods of Use

One aspect of the present invention provides methods of treating virally induced conditions/disease associated with immune suppression (e.g. viral induced tumors or viral infection) in a subject (e.g., human) which includes administering to the subject a therapeutically effective amount of a compound described herein. In one embodiment, the compounds described herein are specifically target against viral replication and/or virally, infected/transformed cells. For example, CMX001 demonstrates specificity against polyomavirus infected cells such as BK virus and JC virus infected cells. (See Example 2 below). In one embodiment, the compounds described herein have a higher cytotoxicity against virally infected and/or transformed cells compared to normal (uninfected cells).

According to one aspect, the compounds described herein may also be used to treat viral induced tumor. Exemplary virus induced tumor is selected from brain cancer, colorectal cancer, nasopharyngeal carcinoma (NPC), Burkitt lymphoma (BL), lymphoma (e.g. B cell lymphoma, Hodgkin's lymphoma, Duncan's syndrome), Merkel cell carcinoma (MCC), prostate cancer, hepatocellular carcinoma, cervical cancer, lung cancer, head and neck squamous cancers, prostate cancer, breast cancer, acute lymphocytic leukemia, adult acute myeloid leukemia, adult non Hodgkin's lymphoma, brain tumors, childhood cancers, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, skin cancer, and a combination thereof. In one embodiment, the viral induced tumor is cervical cancer associated with papillomavirus. In another embodiment, the viral induced tumor is post transplant lymphoproliferative disorder (PTLD) associated with Epstein-Barr virus (EBV).

In some embodiments, the viral induced tumor is associated with polyomavirus (including BK, John Cunningham virus (JCV), Merkel cell virus (MCV), KI polyomavirus (KIV), WU polyomavirus (WUV), Simian virus 40 (SV 40)), papillomavirus (including human papillomavirus, cottontail rabbit papillomavirus, equine papillomavirus and bovine papillomavirus), herpes virus including, but are not limited to, human herpes virus, such as, Herpes simplex virus-1 (HSV-1), Herpes simplex virus-2 (HSV-2), human herpes virus 6 (HHV-6), varicella zoster virus (VZV), Epstein-Barr virus (EBV), lymphocryptovirus, cytomegalovirus (CMV) (including, but is not limited to, human cytomegalovirus (HCMV)), orthopoxvirus, smallpox, cowpox, camelpox, monkeypox, hepatitis B, hepatitis C virus, variola major and minor, vaccinia, ebola virus, papillomavirus, adenovirus or polyomavirus, and a combination thereof.

In one embodiment, viral induced tumor is associated with polyomavirus, papillomavirus, herpes virus, adenovirus, Epstein-Barr virus (EBV), human cytomegalovirus (HCMV), Hepatitis B virus, Hepatitis C virus or a combination thereof. The method of claim 4, wherein said viral induced tumor is associated with papillomavirus selected from the group consisting of human papillomavirus, cottontail rabbit papillomavirus, equine papillomavirus and bovine papillomavirus.

In some embodiments, the viral induced tumor is associated with BK virus or JCV.

In one embodiment, the tumor described herein is colonic pseudotumor associated with cytomegalovirus; glioblastoma associated with cytomegalovirus; gastric cancer associated with Epstien-Barr virus; brain tumor associated with SV(40) or CMV; liver cancer associated with hepatitis B and/or hepatitis C virus; Merkel cell carcinoma associated with Merkel cell polyomavirus; Burkitt's lymphoma, Hodgkin's lymphoma, post-tranplantation lymphoproliferative disease (PTLD) or Nasopharyngeal carcinoma that is associated with EBV; or cancers of cervix, anus or penis that is associated with human papillomavirus.

In one embodiment, the subject is human. In one embodiment, the subject is an immunocompromised subject.

As used herein, immunodeficiency (or immune deficiency) is a state in which the immune system's ability to fight infectious disease is compromised or entirely absent. An immunocompromised subject is a subject that has an immunodeficiency of any kind or of any level. Exemplary immunocompromised subject includes, but are not limited to, a subject with primary immunodeficiency (a subject that is born with defects in immune system) and a subject with secondary (acquired) immunodeficiency In addition, other common causes for secondary immunodeficiency include, but are not limited to, malnutrition, aging and particular medications (e.g. immunosuppressive therapy, such as chemotherapy, disease-modifying antirheumatic drugs, immunosuppressive drugs after organ transplants, glucocorticoids). Other exemplary diseases that directly or indirectly impair the immune system include, but are not limited to, various types of cancer, (e.g. bone marrow and blood cells (leukemia, lymphoma, multiple myeloma)), acquired immunodeficiency syndrome (AIDS) caused by human immunodeficiency virus (HIV), chronic infections and autoimmune diseases (e.g. Acute disseminated encephalomyelitis (ADEM), Addison's disease, Alopecia areata, Ankylosing spondylitis, Antiphospholipid antibody syndrome (APS), Autoimmune hemolytic anemia, Autoimmune hepatitis, Autoimmune inner ear disease, Bullous pemphigoid, Coeliac disease, Chagas disease, Chronic obstructive pulmonary disease, Crohns Disease, Dermatomyositis, Diabetes mellitus type 1, Endometriosis, Goodpasture's syndrome, Graves' disease, Guillain-Barré syndrome (GBS), Hashimoto's disease, Hidradenitis suppurativa, Kawasaki disease, IgA nephropathy, Idiopathic thrombocytopenic purpura, Interstitial cystitis, Lupus erythematosus, Mixed Connective Tissue Disease, Morphea, Multiple sclerosis (MS), Myasthenia gravis, Narcolepsy, Neuromyotonia, Pemphigus vulgaris, Pernicious anaemia, Psoriasis, Psoriatic Arthritis, Polymyositis, Primary biliary cirrhosis, Rheumatoid arthritis, Schizophrenia, Scleroderma, Sjögren's syndrome, Stiff person syndrome, Temporal arteritis (also known as “giant cell arteritis”), Ulcerative Colitis, Vasculitis, Vitiligo, Wegener's granulomatosis.)

In one embodiment, the subject is a transplant patient on immunosuppressive agent. In one embodiment, the transplant patient has at least one transplanted organ selected from the group consisting of kidney, bone marrow, liver, lung, stomach, bone, testis, heart, pancreas and intestine.

In some embodiments, the compounds described herein are applied to subject on immunosuppressive medications, (e.g. transplant patient or subjects that are suffering from an over-active immune system), a subject receiving certain kinds of chemotherapy, or a subject that is infected with human immunodeficiency virus (HIV).

Further, in one embodiment, the viral induced tumor is selected from brain cancer, colorectal cancer, nasopharyngeal carcinoma (NPC), Burkitt lymphoma (BL), lymphoma (e.g. B cell lymphoma, Hodgkin's lymphoma, Duncan's syndrome,), Merkel cell carcinoma (MCC), prostate cancer, hepatocellular carcinoma, cervical cancer and a combination thereof.

According to some aspects of the present invention, for subject in need of an immunosuppressive agent, the compounds described herein may be administered in combination (concurrently or sequentially) with the immunosuppressive agent to treat viral infection and/or viral induced tumors. Any appropriate immunosuppressive agent may be used in combination with compounds described herein. Exemplary immunosuppressive agent is described in section E below.

In one embodiment, the compound having the structure of

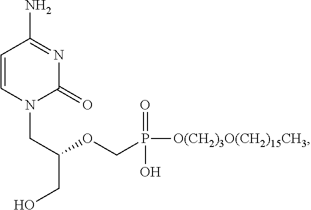

or a pharmaceutically acceptable salt thereof may be administered in combination with at least one immunosuppressant agents that is Tysabri (natalizumab) to treat viral infection and/or viral induced tumors.
E. Combination with Immunosuppressant Agents for Treating Viral Infection or Viral Induced Tumors

The compounds described herein may be used in combination with additional immunosuppressive agents to treat viral infections or viral induced tumors of a subject that in need of immunosuppressant medications. Any immunosuppressive agents known to one skilled in the art may be used in combination with the compounds described herein. Exemplary immunosuppressive agents include, but are not limited to, Daclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm and a combination thereof. In some embodiments, the pharmaceutical composition includes CMX001 and Tysabri (natalizumab).

Additional exemplary immunosuppressant agents are further described in Mukherjee et al., A comprehensive review of immunosuppression used for liver transplantation, Journal of Transplantation, vol. 2009, article ID 701464 and Woodroffe et al., Clinical and cost-effectiveness of newer immunosuppressive regimens in renal transplantation: a systematic review and modeling study, Health Technology Assessment, vol. 9, No. 21 (2005).

### F. Examples

The present invention will now be described in more detail with reference to the following examples. However, these examples are given for the purpose of illustration and are not to be construed as limiting the scope of the invention.

In the following Examples, CMX001 is

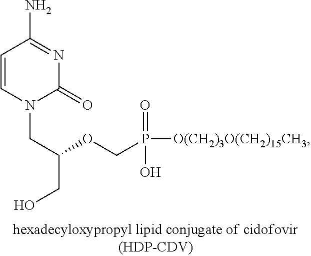

or a pharmaceutically acceptable salt thereof.

In the following Examples, cidofovir is

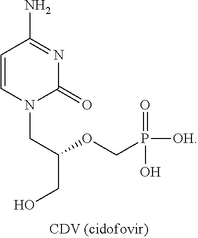

### Example 1

### I Antiviral Activity of CMX001 Against BK Virus

In order to test the activity of CMX001 to inhibit replication of BK virus, stocks of BK virus were prepared in HFF cells and dilutions of the virus stocks were used to infect primary human renal tubular epithelial cells (RPTECs). CMX001 were then added to the wells containing the infected cells and the plates were incubated for 5 days. Total DNA was prepared from the plates and viral DNA was quantified by qPCR.

The in vitro activity of CMX001 against BK virus replication is shown in Table 1. CMX001 exhibited good activity against BK virus in RPTECs. The activity of CMX001 was more potent than cidofovir (See Table 1). The negative control drug, ganciclovir, was essentially inactive.

The assay optimization in the cell line also revealed that the multiplicity of infection appeared to impact the efficacy of cidofovir and CMX001.

TABLE 1
Antiviral activity of CMX001 against BK virus in RPTEC cells

| Virus Dilution | cidofovir EC₅₀ | CMX001 EC₅₀ | ganciclovir EC₅₀ |
|---|---|---|---|
| 1:10 | 2.0 | 0.016 | 89.0 |
| 1:50 | 0.65 | 0.0035 | >100 |
| 1:100 | 0.44 | 0.0037 | 70.8 |

All EC₅₀ values are in μM.

### II Antiviral Activity of CMX001 Against JC Virus

In order to test the ability of CMX001 to inhibit JC virus, COS-7 cells were infected with JCV(Mad-4) at an estimated TCID50 of 0.2. After 2 h incubation at 37° C., supernatants were replaced with fresh medium without or with increasing amounts of CMX001 and incubated for 5 days. JC virus was quantified by qPCR after DNA extraction.

The in vitro data of CMX001 inhibits JC virus replication is shown in Table 2. As shown in Table 2, CMX001 was active against JC virus.

TABLE 2
Antiviral activity of CMX001 against JC virus in COS-7 cells.

| CMX001 EC₅₀ | CMX001 EC₉₀ |
|---|---|
| 0.15 | 0.6 |

All values are in μM.

### Example 2

### I. CMX001 Inhibits Polyomavirus BK Replication in Primary Human Renal Tubular Cells

A. Materials and Methods

Primary human renal proximal tubule epithelial cells (RPTECs), BKV(Dunlop) and all methods as previously described by Bernhoff et al (See Bernhoff et al., Cidofovir inhibits polyomavirus BK replication in human renal tubular cells downstream of viral early gene expression, Am J Transplant 8, 1413-1422 (2008).) Only one exception, quantitative PCR (qPCR) to quantify intracellular or extracellular BKV DNA load was performed with a different primer/probe set also targeting the LTag gene (See Hirsch, et al., J. Prospective study of polyomavirus type BK replication and nephropathy in renal-transplant recipients, N Engl J. Med., 347, 488-496 (2002)). Before each experiment, CMX001 was freshly dissolved to 1 mg/ml in methanol/water/ammonium hydroxide (50/50/2). It was further diluted in RPTEC growth medium.

B. Experiments and Results

(1) Determination of Inhibitory Concentration IC₉₀

To investigate the effect of CMX001 on BKV progeny, increasing concentrations of CMX001 were added 2 h p.i. and supernatants harvested at 72 h p.i. It was observed that CMX001 reduced the extracellular BKV load in a concentration dependent manner (See FIG. 1 a). When viral input was subtracted, CMX001 0.31 μM reduced the BKV load by an average of 90% defining the inhibitory concentration IC₉₀. Immunofluorescence staining 72 h p.i. of BKV-infected RPTECs demonstrated decreasing numbers of BKV-infected cells with increasing CMX001 concentration (FIG. 1 b). With CMX001 0.31 uM an approximately 60% decrease in BKV agnoprotein expressing cells was seen. The number of cells expressing LTag was less reduced but the signal intensity was lower than in untreated cells. With 2.5 uM CMX001 only few cells were positive for agnoprotein and LTag expression but the total number of cells in the well appeared to be reduced. With 5 uM CMX001 only few weakly LTag stained cells but no agnoprotein expressing cells were observed with a more pronounced effect on total cell number. With 10 uM CMX001 no BKV-infected cell was observed and the total cell number was even more reduced. The conclusion is that CMX001 reduced the expression of early and late BKV proteins and the production of extracellular progeny but also seemed to affect the proliferation rate of RPTECs at higher concentrations.

(2) Effects of CMX001 on RPTECs (DNA Replication and Metabolic Activity)

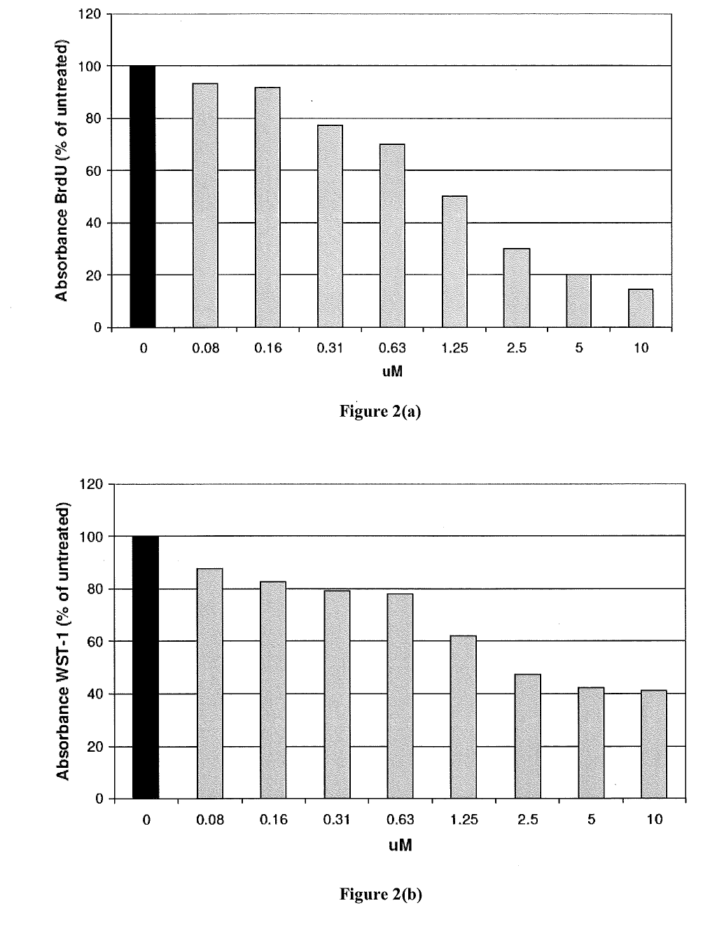

Inspection of RPTECs by phase contrast microscopy did not reveal any signs of impaired viability during the 3 day exposure to CMX001 0.31 μM. To use more sensitive assays, host cell DNA replication and metabolic activity using BrdU incorporation and WST-1 assays in uninfected RPTECs were used. Addition of CMX001 reduced both DNA replication (FIG. 2a) and metabolic activity (FIG. 2b) of uninfected RPTECs in a concentration-dependent manner. Compared to untreated RPTECs, CMX001 at 0.08 to 10 μM decreased DNA replication by 15% to 93% and the metabolic activity by 41% to 88%, respectively. At the concentration of 0.31 μM which caused a 90% inhibition of BKV replication, it is observed an approximately 20% reduction in BrdU incorporation and WST-1 activity.

(3) Effect of CMX001 on BKV Genome Replication

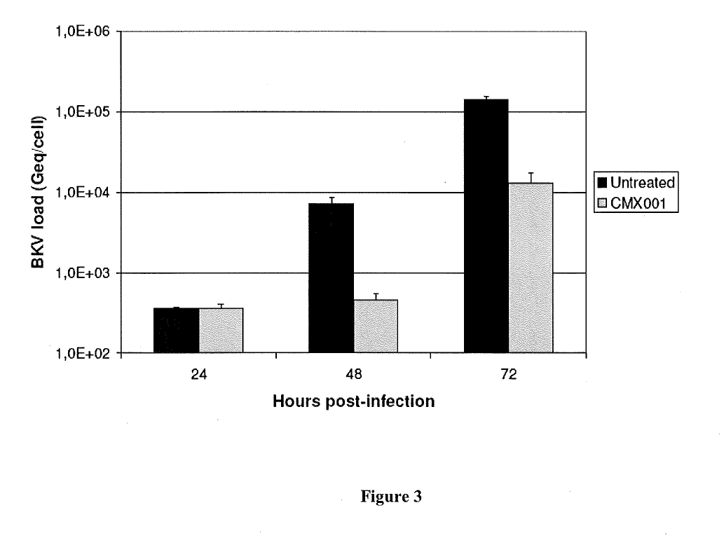

To investigate whether the BKV genome replication was affected by CMX001, intracellular BKV load at 24-72 h p.i. by qPCR was measured. The intracellular BKV load was normalized to the cell number using the aspartoacylase (ACY) gene as described (See Bernhoff et al., 2008; Randhawa, et al., Quantitation of DNA of polyomaviruses BK and JC in human kidneys. J Infect Dis., 192, 504-509 (2005)). Compared to untreated RPTECs, CMX001 0.31 μM reduced the intracellular BKV load by 94% at 48 h and 63% at 72 h p.i. (FIG. 3). Then, a significant inhibitory effect of CMX001 on intracellular BKV genome replication, which is the second step of the BKV lifecycle may be identified. This step is known to require LTag expression which also increases viral late gene expression by two mechanisms: 1. increasing the DNA templates for late gene transcription and 2. by activating transcription from the late promoter (Cole, C. N., Polyomavirinae: The Viruses and Their Replication. In Fields Virology, Third edn, pp. 1997-2043. Edited by B. N. Fields, D. M. Knipe & P. H. Howley. New York: Lippincott-Raven (1996).)

(4) CMX001's Effects on BKV Early and Late Gene Expression

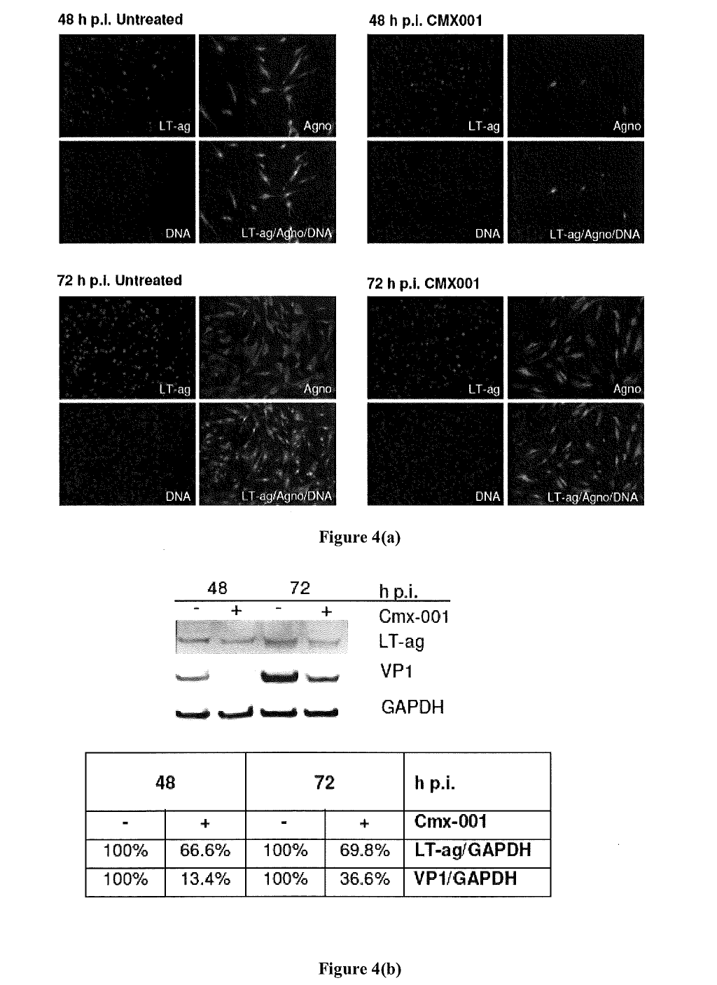

To investigate expression of LTag at the single-cell level, immunofluorescence staining at 48 and 72 h p.i was performed. At 48 h p.i. the number of BKV positive cells was almost the same in CMX001 and untreated wells. At 72 h p.i., the CMX001 treated cells seemed to express less LTag pro cell (FIG. 4a). When late protein expression at 48 and 72 h p.i was examined by immunofluorescence staining, a significant reduction of agnoprotein (FIG. 4a) and VP1 (data not shown) was observed in CMX001-treated RPTECs. By western blotting the decrease of VP1 was found to be 86% and 63% at 48 and 72 h p.i., respectively (FIG. 4b) while LTag staining was found to be 33% and 30% reduced at 48 and 72 h p.i., respectively. Interestingly, immunofluorescence also revealed some refractory cells in the CMX001 treated culture expressing agnoprotein at levels comparable to untreated cells even with CMX001 concentrations up to 2.5 μM. It was concluded that CMX001 significantly reduces late protein expression but also inhibit early protein expression at late time points of infection.

(5) Timecourse of CMX001 on Extracellular BKV Load

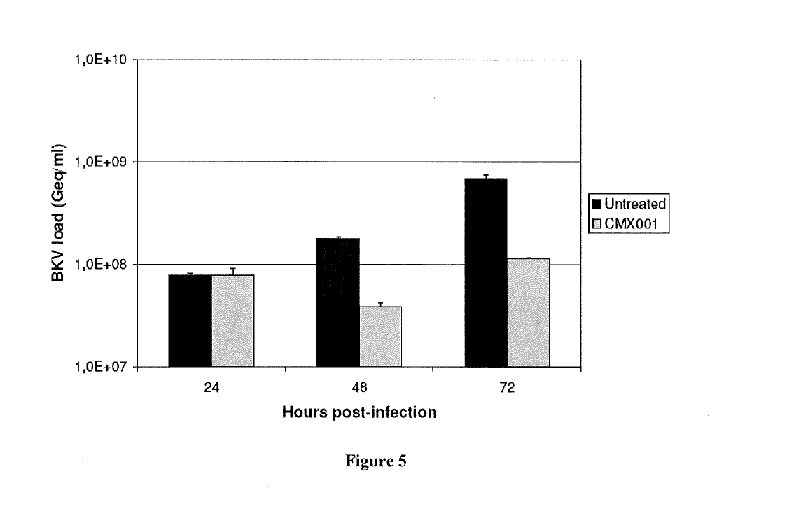

To examine the effect of CMX001 on BKV progeny over time, supernatants of treated and untreated cells were harvested at the indicated timepoints. As earlier described (Bernhoff et al., 2008), the completion of the first lifecycle of BKV(Dunlop) in untreated RPTECs take between 48 and 72 h. While an increased BKV load was observed in supernatants from untreated cells at 48 h p.i, only input virus could be detected in CMX001 treated cells 48 h p.i. At 72 h an increased viral load was seen in both untreated and CMX001 treated cells but the BKV loads in supernatants from CMX001 treated cells were 84% lower (1.13×10⁸ Geq/ml) than in untreated cells (FIG. 5). It is concluded that progeny production in CMX001 treated RPTECs may be delayed.

(6) Treatment of RPTECs before Infection

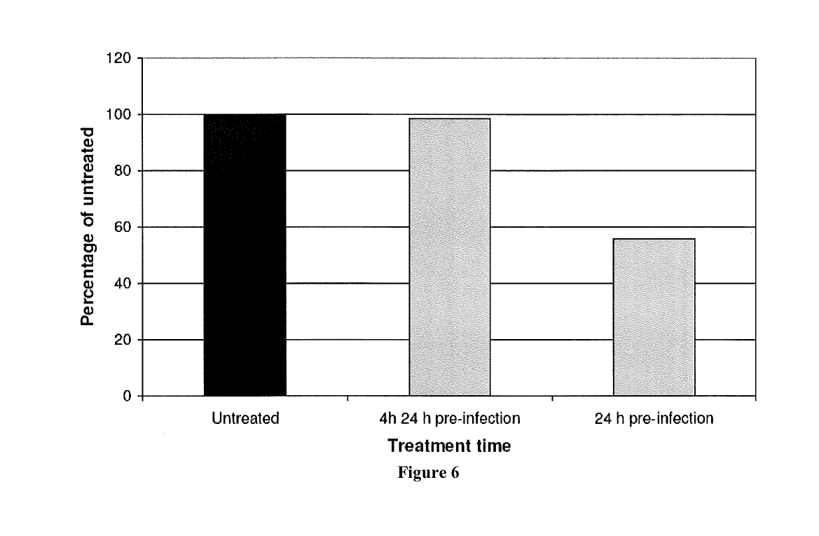

To investigate whether or not pre-treatment of cells before virus inoculation could inhibit BKV-infection, RPTECs were either treated for 4 hours and CMX001 was replaced by complete growth medium 20 h pre-infection, or cells were treated for 23 hours at 24 h pre-infection but CMX001 was replaced at one hour before infection with complete growth medium. While treatment for 4 hours, ending 20 hours before infection, had hardly any effect on the BKV load 72 h p.i., treatment for 23 hours until one hour before infection did reduce the viral load by about 50% (FIG. 6). Thus, CMX001 pre-treatment does reduce but not prevent BKV replication.

(7) Stability of CMX001

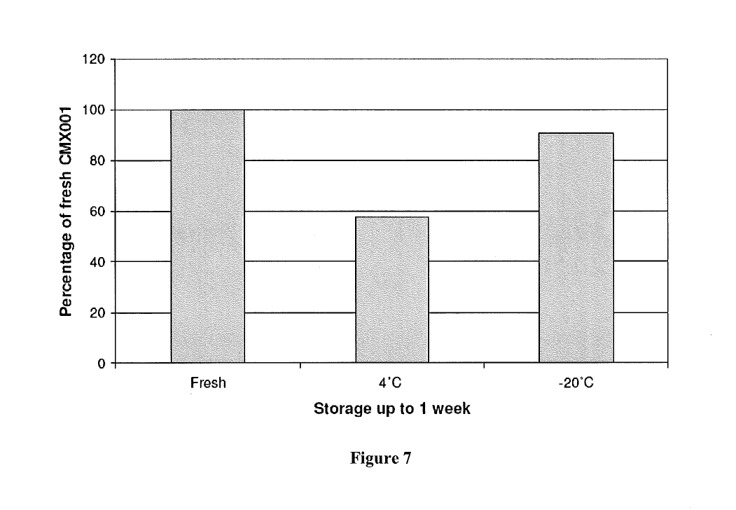

To examine the stability of CMX001 stock solution 1 mg/ml was put in 4 or −20° C. for one week then diluted to 0.31 uM and tested for its antiviral effect by measuring the extracellular BKV load in BKV-infected RPTEC 3 d p.i. Drug stored at 4° C. had less than 60% activity while drug stored at −20° C. had an approximately 90% activity compared to the freshly prepared drug (FIG. 7).

C. Discussion

The preliminary results from treating BKV-infected RPTECs with CMX001 were shown in FIG. 1-7 as well as the above discussion related to FIGS. 1-7. CMX001 inhibits BKV-infection at the level of BKV genome replication at about 400 times lower concentrations than CDV (CDV 40 ug/ml=127 uM versus CMX001 0.31 uM). CMX001 at a concentration of 0.31 uM reduced extracellular BKV loads by approximately 90% defining the IC90. The same CMX001 concentration decreased cellular DNA replication in uninfected cells by 22% and metabolic activity by 20%. However, in some previous research (Bernhoff et al., 2008) it is shown that BKV infection increase cellular DNA replication by about 40% and metabolic activity round 20% and therefore the hypothesis is that both DNA replication and metabolic activity will be at the level of uninfected cells when CMX001 0.31 uM is used to treat BKV-infected cells.

CMX001 at 0.31 uM reduce BKV DNA replication by 94% at 48 h p.i. At the same time VP1 expression is 86% reduced. However at 72 h p.i., the decrease in DNA replication is only 63%. This discrepancy requires further studies including the effect on infectious supernatants.

When extracellular BKV loads were measured at 24, 48, and 72 h p.i., only input was detectable in CMX001 treated cells at 48 h p.i. indicating that very little or no virus is released before 48 h p.i. At 72 h p.i, only a minor increase in the BKV load was observed accounting for a 84% reduction compared to untreated cells.

Experiments to examine the effect of pre-treatment of RPTEC with CMX001 prior to infection could prevent BKV replication were conducted and results are shown above. Pre-treatment for 4 h 24 h pre-infection did not inhibit BKV replication. However, pre-treatment for 24 h until one hour before infection reduced the viral load about 50%. Thus pre-treatment of cells will partly inhibit BKV replication.

Comparing the immunofluoresence staining of CMX001 IC₉₀ treated cells 72 h p.i and CDV IC₉₀ treated cells (Bernhoff et al., 2008), LT-ag expression 72 h p.i. seem to be more reduced by CMX001 than by CDV. As for CDV, treatment refractory cells were present even at CMX001 concentrations 8 times higher than IC₉₀. Since CMX001 does not depend on the organic anion transporter, selective expression of this transporter in the cells can not explain the phenomena.

For each CMX001 experiment, fresh stock solutions were prepared. This could lead to minor concentration variation from experiment to experiment. The effect of storing CMX001 stock solutions was therefore tested. Storage of CMX001 at one week at 4° C. or at −20° C. decreased the antiviral activity. However, the possibility that different activity of the stored and freshly prepared CMX001 could be due to minor concentration differences in the stock ca not be excluded.

The conclusion is that the IC₉₀ of CMX001 against BK virus replication in primary human renal proximal tubule epithelial cells was 0.31 M. In uninfected cells, CMX001 at 0.31 μM inhibited metabolic activity and DNA replication by approximately 20%.

In addition, CMX001 like CDV inhibits BKV replication in primary human RPTECs downstream of initial LT-ag expression. Probably due to a more favourable uptake, the IC₉₀ for CMX001 in RPTERCS is 410 times lower than for CDV. The host cell toxicity seems to be comparable to CDV. A clear advantage of CMX001 in BKV treatment is the possible oral administration.

### II. Inhibition of polyomavirus JC replication by CMX001

A. Material and Methods

(1) Cell Culture

COS-7 cells were grown in DMEM-5%. Astrocytes derived from progenitor cells were maintained in MEM-B-10% supplemented with Gentamycin. JCV Mad-4 (ATCC VR-1583) supernatants from infected COS-7 cells with a TCID50 of 104.5 per ml were used for infection of cultured cells.

(2) Infection and CDV-Treatment

COS-7 or astrocyte cells were infected at a confluence of 60-70% with JCV(Mad-4) at an estimated TCID50 of 0.2. After 2 h incubation at 37° C., supernatants were replaced with fresh medium without or with increasing concentration CMX001. CMX001 was freshly dissolved to 1 mg/ml in methanol/water/ammonium hydroxide (50/50/2) and then further diluted the respective growth medium.

(3) Transfection of JCV Genome

Religated JCV Mad-4 DNA was transfected into 50% to 70% confluent COS-7 cells by using Lipofectamine 2000 (Invitrogen) according to the manufacturer's instructions at a DNA:Lipid ratio of 0.8:1.

(4) Immunofluorescence

Cells were fixed with 4% p-formaldehyde (PFA) in phosphate-buffered saline, pH6.8 (PBS) at room temperature for 20 min and permeabilized with 0.2% Triton X-100 in PBS at room temperature for 10 min. After washing twice with PBS at room temperature for 5 min, PFA was quenched with 0.5 M NH4Cl in PBS at room temperature for 7 min followed washing twice with PBS at room temperature for 5 min. Blocking of unspecific binding sites was done with 3% milk in PBS at 37° C. for 15 min. Primary antibody (rabbit anti-VP1 1:300 in 3% milk PBS) was incubated at 37° C. for 45-60 min. Cells were washed twice with PBS on a shaker at room temperature for 5 min. Secondary antibody (chicken anti-rabbit Cy3 1:2000) and 5 μg/ml Höchst 33342 dye to stain DNA was given to the cells then incubated at 37° C. for 45-60 min. Cells were washed twice with PBS as before. Coverslips were mounted in NPropylgallat.

(5) Real Time PCR

JCV loads were quantified after DNA extraction from 100 ul cell culture supernatants and the Corbett X-tractor Gene and the Corbett VX reagents (Qiagen, Hombrechtikon, Switzerland). The real-time PCR protocol for detection of JCV DNA samples targets the JCV large T coding sequence and has been described elsewhere (5).

(6) WST-1 Assay

The metabolic activity was monitored by the colorimetric WST-1 assay (Roche) of the mitochondrial dehydrogenases in viable cells. COS-7 cells were seeded in 96 well plates and CMX001 was added at indicated concentrations. The WST-1 cleavage product was measured at 450 nm (sample) and at 650 nm (background). WST-1 plus medium alone served as blank.

(7) BrdU Assay

DNA synthesis was quantified by the colorimetric measurement of BrdU incorporation into DNA in proliferating cells using the ‘Cell proliferation ELISA, BrdU’ kit (Roche). COS-7 cells were seeded in 96 well plates and CMX001 was added at indicated concentrations. Absorbance at 450 nm (sample) and at 650 nm (background) was determined 2 h after addition of the substrate.

B. Experiments and Results

(1) Replication of JCV Mad-4 in Cell Culture

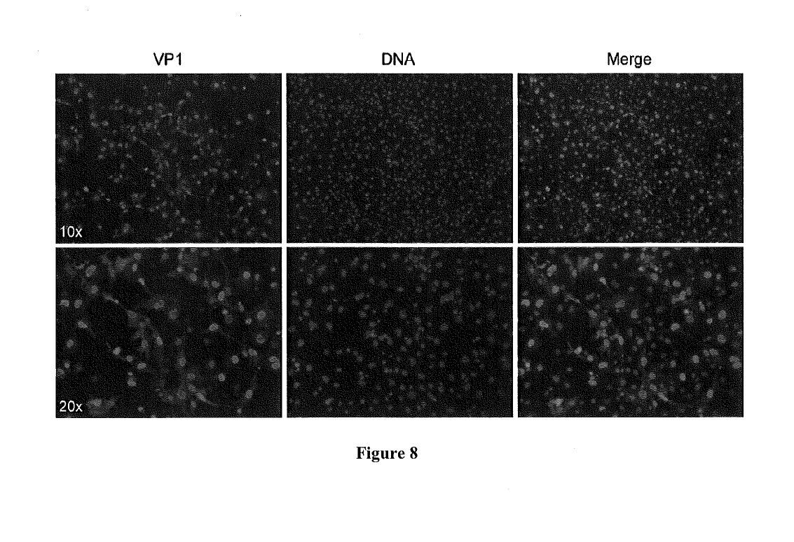

The replication characteristics of JCV Mad-4 in COS-7 and astrocyte cell cultures were firstly investigated. At 7 days post-infection (d.p.i.), JCV-infected COS-7 cells were fixed and stained by indirect immunofluorescence. As shown in FIG. 8, JCV late viral capsid protein VP1 is detectable as red signal suggesting that JCV is completing the viral life cycle in COS-7 cells (FIG. 8, left panel). The counter stain for DNA with Höchst-33342 marked the nuclei in blue (FIG. 8, middle panel). Merging both pictures (FIG. 8, right panel) indicated that JCV Mad-4 VP1 is present in the nucleus of the infected COS-7 cells. At a higher magnification, the VP1 signal was dispersed throughout the entire nucleus, but sparing the nucleoli (FIG. 8, left panel). Cells showing an intense VP1 signal in the nucleus had a diffuse staining pattern in the cytoplasm as well. JCV-infected cells showed enlarged nuclei (FIG. 8, middle panel) compared to uninfected cells present in the same cell culture (FIG. 8, right panel). The data demonstrate that COS-7 cells are susceptible to JCV Mad-4 infection and that about 30% of cells have entered the late phase of the JCV lifecycle at 7 d.p.i.

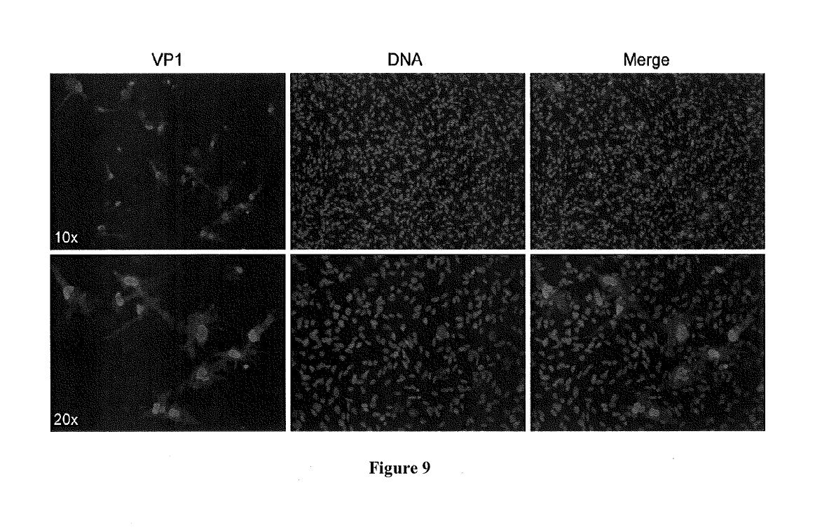

Similar experiments infecting astrocyte cells with JCV Mad-4 were performed. At 7 the subcellular distribution of JCV VP1 appeared similar to JCV Mad-4 infected COS-7 cells (FIG. 9, left panel, red). The counterstain for DNA with Höchst-33342 marked the astrocyte cell nuclei in blue (FIG. 9, middle panel). Merging both pictures indicated that JCV-Mad4 VP1 is present in the nucleus of the infected astrocyte cells. Comparison with the JCV Mad-4 infection of COS-7 cells, significantly fewer astrocyte cells were positive for the late protein VP1 (FIG. 9, right panel). At higher magnification, the VP1 signal was found mainly in the nucleus sparing the nucleoli and a rather diffuse pattern in the cytoplasm of the astrocyte cells (FIG. 9, left panel). Staining of the DNA indicated that large nuclei are present in the culture (FIG. 9, middle panel), which belong to JCV-infected cells (FIG. 9, right panel). Astrocyte cells were also stained for the viral early protein large T-antigen (LT) as expected, the LT was located in the nuclei of infected cells (data not shown). All cells positive for late protein VP1 also expressed LT, but few astrocyte cells were only positive for LT. This observation indicated that JCV Mad-4 proceded through the polyomavirus life cycle as expected. The confluency at 7 days p.i indicated that astrocytes are slow growing cells and, thus, JCV replication is prolonged compared to BKV replication in human renal proximal tubular epithelial cells. Our observations suggest that a 7 day growth period is not optimal for efficient JCV replication.

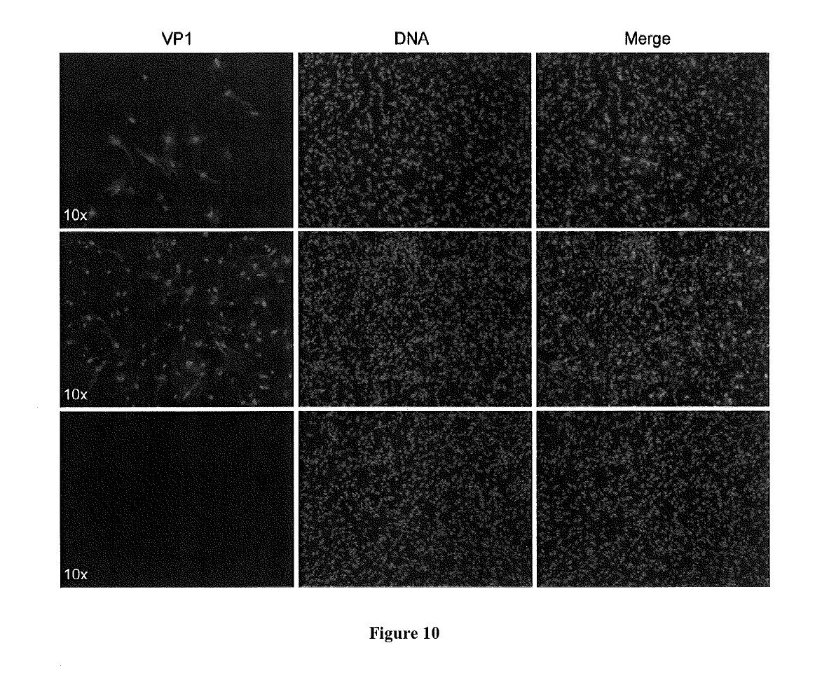

Given the slower replication rate of JCV Mad-4 in astrocyte cells, the state of infection at 14 d.p.i. was examined. The results indicated that the number of cells positive for the early LTag (data not shown) and late VP1 had increased by approximately 4-fold (FIG. 10). Thus, the astrocyte cells supported JCV Mad-4 replication, but the life cycle seemed to be considerably prolonged compared to the one observed in COS-7 cells.

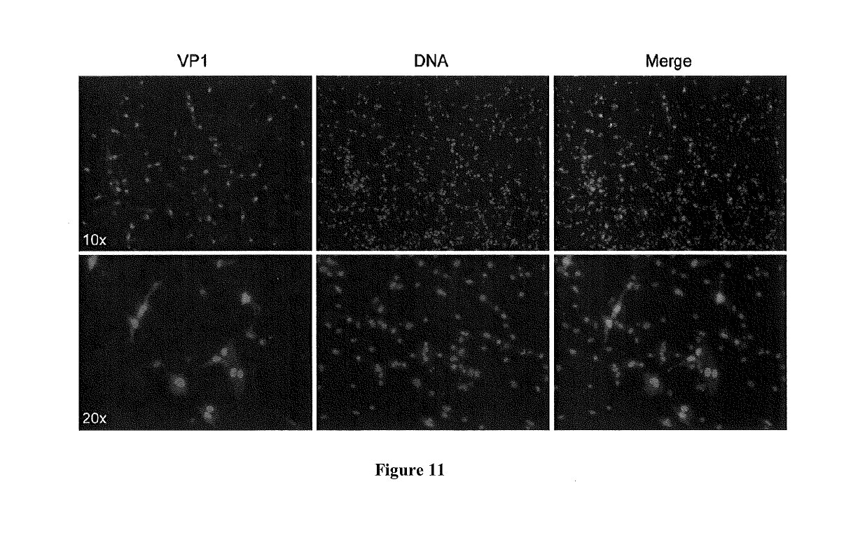

Next, the replication competence of religated JCV Mad-4 DNA transfected into COS-7 cells was tested. This approach would be a helpful tool to perform reverse genetics to test CMX001 efficacy on JCV variants. As shown in FIG. 11, JCV late viral capsid protein VP1 is detectable as red signal indicating that JCV is replication competent in COS-7 cells after 7 days post transfection, d.p.t. (FIG. 11, left panel). The counterstain with Höchst 33342 dye for DNA marked the nuclei in blue (FIG. 11, middle panel). Merging both pictures (FIG. 11, right panel) indicated that JCV Mad-4 VP1 is present in the nucleus of the transfected COS-7 cells. At a higher magnification, the VP1 signal was dispersed throughout the entire nucleus, but sparing the nucleoli (FIG. 11, left panel). Cells showing an intense VP1 signal in the nucleus had a diffuse staining pattern in the cytoplasm as well. JCV-transfected cells showed enlarged nuclei (FIG. 11, middle panel) compared to normal cells present in the same cell culture (FIG. 11, right panel). The data demonstrate that COS-7 cells are susceptible to JCV Mad-4 DNA transfection and that about 15% of cells have entered the late phase of the JCV lifecycle by day 7 p.i. After transfection, the subcellular staining pattern for late protein VP1 was identical to the VP1 staining after infection with JCV Mad-4.

(2) Determination of Inhibitory Concentration IC₉₀

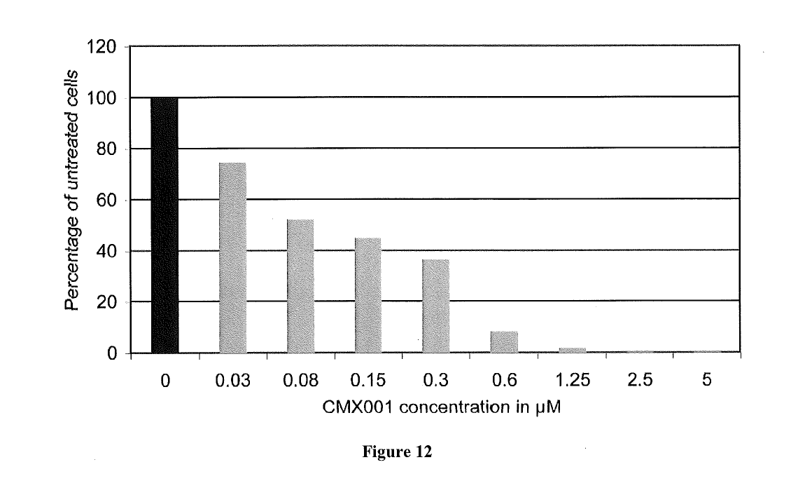

To investigate the effect of CMX001 on JCV progeny, increasing concentrations of CMX001 were added at 2 h p.i. and supernatants harvested at 5 d.p.i. It was observed that CMX001 reduced the extracellular JCV load in a concentration dependent manner (FIG. 12). Between day 1 and 5 p.i., the viral load increased in untreated cells by about 2.5 log (1.24×107 vs 5.09×109). By contrast, in cells treated with 2.5 μM CMX001, it is observed only 2½-fold increase during the same time period (9.69×106 vs 2.44×107). Subtracting the viral input defined as the viral load at day 1 p.i., CMX001 at 0.15 μM reduced the JCV viral load by >50% and 0.6 μM reduced JCV by >90%, defining the inhibitory concentration IC-₅₀ and IC-₉₀ at day 5 respectively.

(3) Effects of CMX001 on COS-7 Cell Metabolic Activity

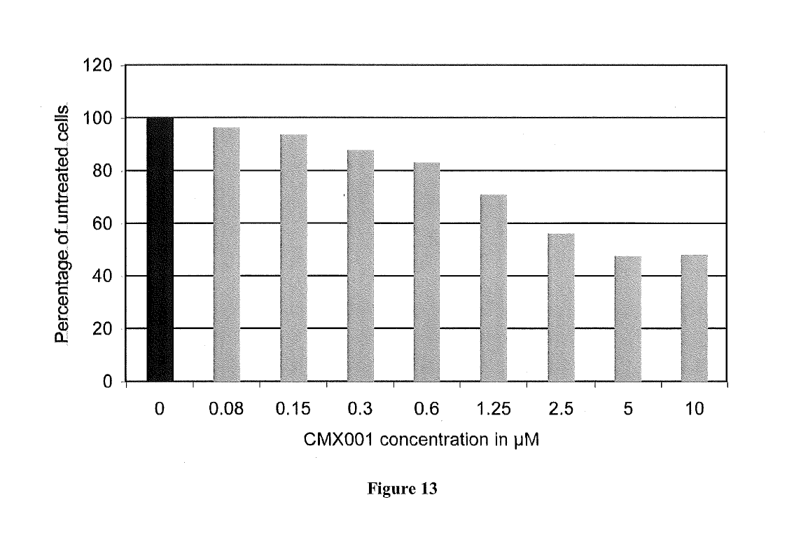

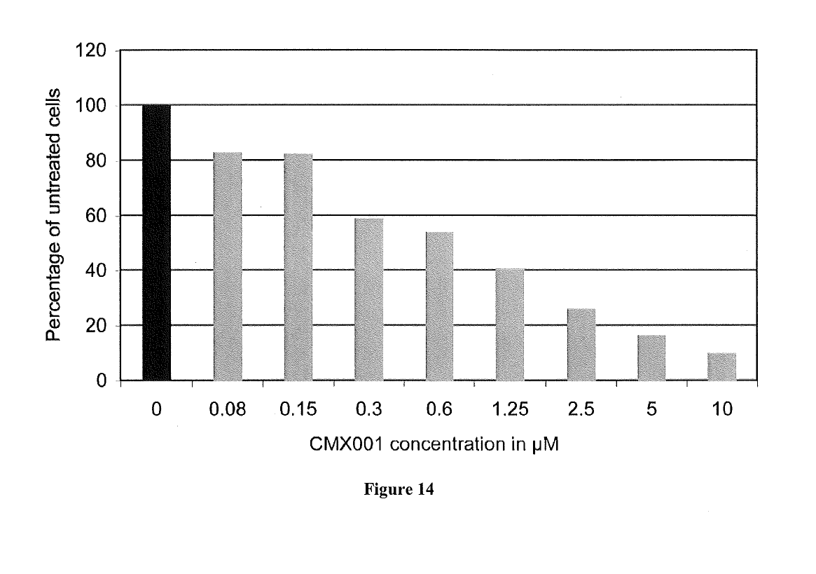

Inspection of COS-7 by phase contrast microscopy did not reveal any signs of impaired viability during the 7 day exposure to CMX001 0.6 μM. To use a more sensitive assay, the effects on the cellular metabolic activity using WST-1 assay and BrdU incorporation in uninfected COS-7 cells was investigated. Addition of CMX001 reduced the metabolic activity (FIG. 13) and DNA replication (FIG. 14) of uninfected COS-7 cells in a concentration-dependent manner. Compared to untreated COS-7 cells, increasing CMX001 concentrations from 0.08 to 10 μM decreased the metabolic activity from 3% to 52% and DNA replication 83% to 10%, respectively. At 0.6 μM, COS-7 cells showed a modest loss of metabolic activity of 17%, an approximately 50% reduced BrdU incorporation. Taken together, CMX001 significantly reduced host cell metabolic activity and DNA replication at higher concentrations. Similar experiments were also conducted with astrocyte cells at CMX001 concentrations of 0.08 to 5 μM. DNA replication in uninfected astrocytes decreased by 25% to 92% at the highest CMX001 concentration (data not shown). Comparing the CMX001 associated inhibition of DNA replication both cell types, it seemed that COS-7 were slightly less sensitive (83% vs 92%, respectively). However, for astrocyte cells, the 2 h substrate incubation period of the assay seemed to be not optimal since the optical density was low compared to the readings for COS-7. This is consistent with our observation that astrocyte cells had a slower metabolism compared to COS-7 cells.

(4) CMX001 Effects on Extracellular JCV Load in COS-7

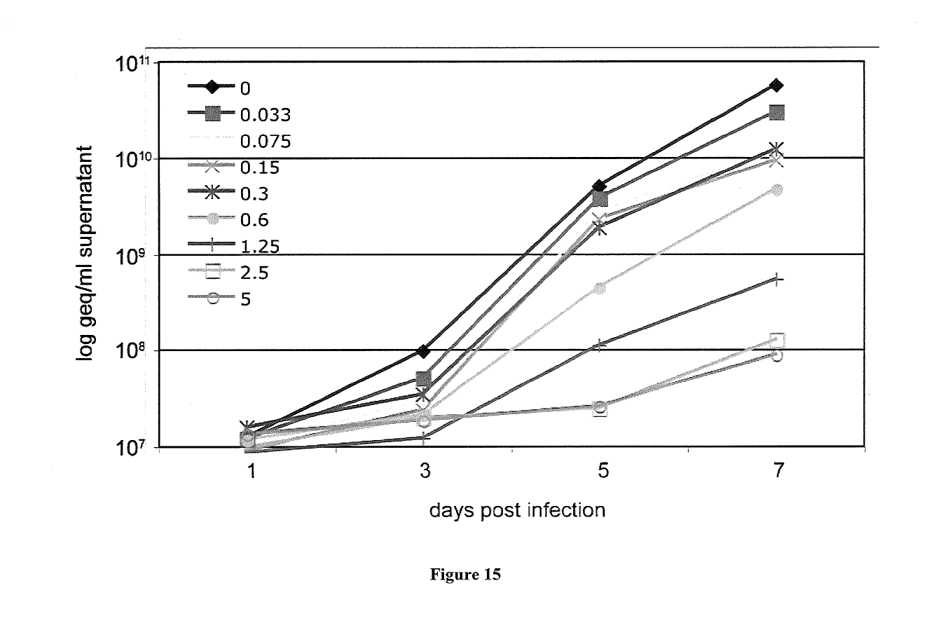

To examine the effect of CMX001 on JCV progeny levels over time, supernatants of treated and untreated cells were harvested at the indicated time points. Supernatants at day 1 p.i. were taken as a measure of input virus. In untreated cells, the extracellular JCV load increased by more than 3 logs over the observation period of 7 days (1.24×107 vs 5.67×1010 geq/ml). In the presence of 5 nM CMX001, it is observed only a modest change in the JCV load of less than 1 log (1.34×107 vs 8.85×107 geq/ml (FIG. 15). When JC viral loads were compared for CMX001 5 uM treated cells at 7 d.p.i. with virus progeny in untreated cells at 3 d.p.i., the viral load in the CMX001 treated cells reached only 91% of the (9.72×107 vs 8.85×107 geq/ml). It was concluded that progeny production in CMX001 treated COS-7 cells may be delayed.

(5) CMX001 Reduces JCV Late Gene Expression

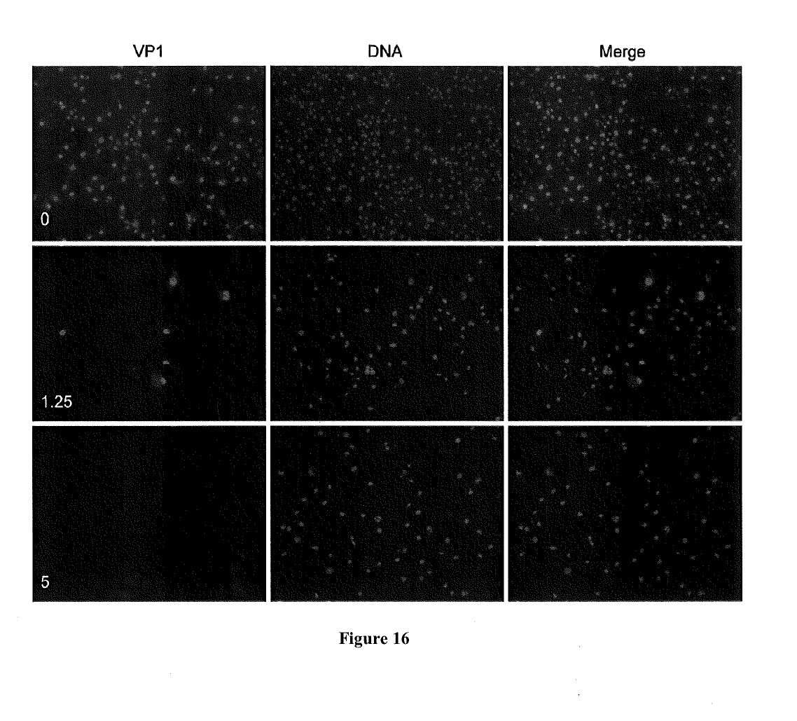

To investigate the effect of CMX001 on late gene expression, immunofluorescence staining was performed for JCV VP1. At 7 d.p.i., untreated COS-7 cells were stained for JCV VP1 as comparison (FIG. 16, upper panel). Addition of CMX001 at 1.25 μM was associated with a significant reduction of JCV VP1 signally (FIG. 16, middle panel). At the highest CMX001 concentration of 5 μM, essentially no VP1 positive cells could be observed by immunofluorescense (FIG. 16, lower panel). It was concluded that CMX001 significantly reduces JCV late protein expression between 1.25 μM and 5 μM.

(6) CMX001 Effects on Extracellular JCV Load in Astrocyte Cells

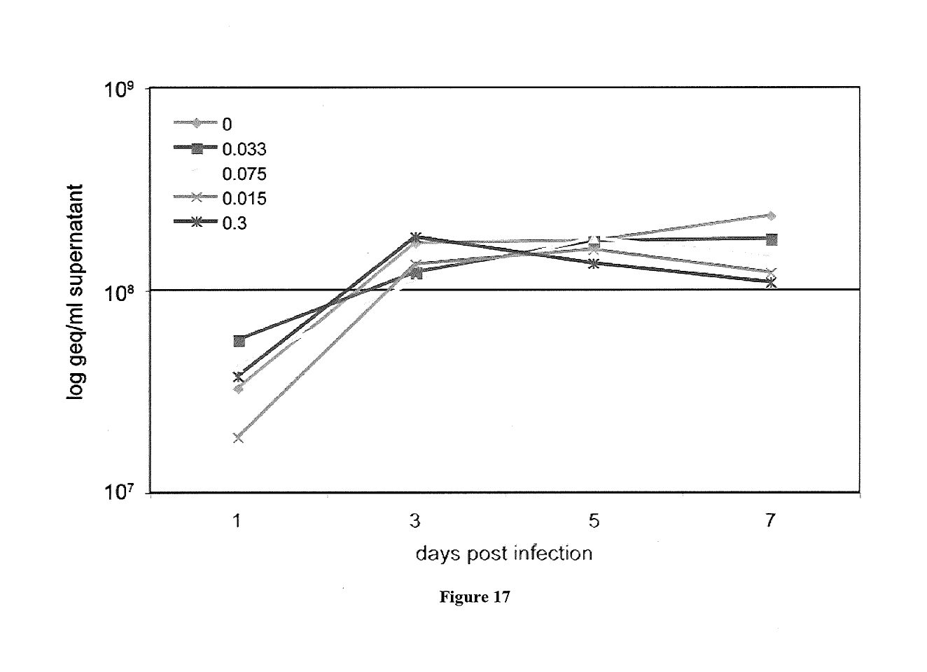

To examine the effect of CMX001 on JCV progeny levels over time, supernatants of treated and untreated cells were harvested at the indicated timepoints. To determine input virus samples were taken at 1 d.p.i. In the course of the infection for untreated cells, the extracellular JCV load changes approximately 1 log over the period of 7 days (3.22×107 vs 2.30×108 geq/ml). In the presence of CMX001, JCV load of less than 1 log was seen (1.34×107 vs 8.85×107 geq/ml (FIG. 17). It was concluded that JCV replication was significantly slower in astrocyte cells than in COS-7. Despite the tendency of low concentrations of CMX001 to inhibit progeny production, the observation period of 7 days did not allow to measure inhibitory effects of CMX001.

### C Discussion

The preliminary results suggest that CMX001 inhibits JCV replication in COS-7 cells. The CMX001 concentration of 0.6 μM reduced extracellular JCV loads by approximately 90%. This concentration is 2 orders of magnitude lower than concentrations reported for CDV inhibition, but in the same range as observed for BKV. Here, the CMX001 IC-90 of BKV replication was determined as 0.31 μM in primary tubular epithelial cells (34). It was observed that CMX001 decreased the host cell metabolic activity by 17% and DNA replication by about 50%. When extracellular JCV loads were measured from 1 to 7 d.p.i., the JCV load from cells treated with the highest concentration of CMX001 (5 μM) was only slightly higher at 5 d.p.i. than input virus at 1 d.p.i. indicating that very little or no virus is released within 4 days. At 7 d.p.i, there was only a minor increase in the JCV load at this concentration, and the level of progeny virus was below the viral load measured at 3 d.p.i. of untreated cells. In astrocyte cells, there is a trend that low concentration of CMX001 might delay the accumulation of progeny virus. However, the inefficient progeny virus production in untreated astrocyte cells 7 d.p.i. demonstrated that the JCV replication cycle is significantly slower.

The difference between COS-7 and astrocyte cells is likely due to the transformed phenotype of COS-7 cells including the expression of the SV40 large T-antigen supporting a more efficient replication cycle of JCV. In astrocyte cells, this is considerably slower.

The conclusion is that The IC₅₀ and IC₉₀ values for CMX001 against JC virus replication in vitro in COS-7 cells was 0.15 and 0.6 μM, respectively. In uninfected COS-7 cells, the IC₅₀ of CMX001 for metabolic activity and DNA replication was approximately 5 and 0.6 μM, respectively. Notably, these cells express polyomavirus T antigen may be specifically sensitive to the effects of CMX001.

### Example 3

### Examples of Using CMX001 on Human Patients with EBV-Associated Intracranial Post-Transplant Lymphoproliferative Disorder (PTLD)

The first patient is a 11-year-old patient with a history of sickle cell anemia developed EBV-associated intracranial post-transplant lymphoproliferative disorder (PTLD). EBV was positive in the plasma (7 Dec. and 14 Dec. 2010) and brain biopsies were consistent with PTLD. In early December, the patient presented with a 3 day history of persistent headache, nausea, vomiting, and diarrhea. The patient was admitted to the hospital and had an acute episode of severe headache with possible seizure activity. A CT of the brain showed a ring-enhancing mass in the right frontal lobe and brain biopsy was consistent with EBV-associated PTLD. The patient was admitted to PICU. High intracranial pressure, repetitive seizures associated with apnea led to intubation and emergency request for CMX001. The use of CMX001 in this patient with EBV-associated PTLD is ongoing since 26 Dec. 2009. The patient has tolerated CMX001 well, and continues to receive 4 mg/kg twice weekly. She has had clinical improvement of her signs and symptoms of disease as well as stabilization if not reduction of her intracranial mass. EBV in the plasma remains negative.

The second patient was a 6 month old heart transplant recipient with EBV-associated PTLD. The patient acquired a primary EBV infection post-operatively. PET scans showed lesions in the liver, lung, and bone (iliac crest) consistent with PTLD. The clinical condition continued to destabilize with what was presumed to be EBV-associated encephalitis with EBV detected in the CSF, clinical and EEG-correlated seizure activity and decreased responsiveness and changes in mental status. At the time of request for CMX001, the patient was on mechanical ventilation, had evidence of both pneumonia and PTLD of his lungs, evidence of seizure activity with clinical criteria for encephalopathy being present. The patient received his first dose of CMX001, 20 mg (approximately 3.3 mg/kg) on 3 Mar. 2010, and his second dose on 7 Mar. 2010 via NG tube The patient had a significant decline in EBV viral loads from 267,338 copies/mL (3 Mar. 2010) to 47,427 (7 Mar. 2010). The patient continued to show evidence of progressive neurologic injury. The parents withdrew support on the day following his second dose of CMX001.

### Example 4

### I. Non-GLP In Vitro Evaluation of CMX001 Against a Panel of Human Tumor Cell Lines

The cell growth inhibitory activities of CMX001, Cidofovir, and a standard agent (varies depending on the tumor type of the cell lines) were evaluated in a panel of cell lines derived from multiple tumor types using a MTS or SRB based cell proliferation assay.

The compounds showed varying degrees of inhibitory activity against the cell lines. CMX001 was more potent than its parent compound, Cidofovir, in almost all 24 cell lines evaluated in the study. The IC₅₀ values of CMX001 in the tumor cell lines ranged from 6.6 μM to 100 μM, which are relatively moderate when compared to those of the standard agents.

### I Materials and Methods

### (1). Cell Lines:

The cell lines used in the test are listed in Table 3. Cells were supplied with fresh media every 2-3 days and passed every 3-5 days depending on their growth rates. After 10-15 passages, cells were discarded and a fresh culture was established from frozen stocks. Cell cultures were checked for mycoplasma and other contaminants regularly.

TABLE 3
Cell Lines used in the study

| Cell | Tissue | | | |
|---|---|---|---|---|
| line | Type | Histology | Source | Growth Media |
| Caki-1 | Kidney | Renal Cell | ATCC | McCoy's 5A |
| | | Carcinoma | | (10% FBS) |
| Caki-2 | Kidney | Renal Cell | ATCC | McCoy's 5A |
| | | Carcinoma | | (10% FBS) |
| SCaBER | Bladder | SCC | ATCC | MEM (10% FBS) |
| SW 780 | Bladder | Carcinoma | ATCC | Leibovitz's |
| | | | | L-15 (10% FBS) |
| DU 145 | Prostate | Carcinoma | ATCC | RPMI (10% FBS) |
| PC-3 | Prostate | Adenocarcinoma | ATCC | RPMI (10% FBS) |
| MDA-231 | Breast | Adenocarcinoma | ATCC | RPMI (10% FBS) |
| MCF-7 | Breast | Adenocarcinoma | ATCC | RPMI (10% FBS) |
| PANC-1 | Pancre- | Carcinoma | ATCC | RPMI (10% FBS) |
| | atic | | | |
| MIA | Pancre- | Carcinoma | ATCC | RPMI (10% FBS) |
| PaCa-2 | atic | | | |
| HT-29 | Colon | Adenocarcinoma | ATCC | RPMI (10% FBS) |
| HCT-116 | Colon | Adenocarcinoma | ATCC | RPMI (10% FBS) |
| H460 | Lung | NSCL | ATCC | RPMI (10% FBS) |
| A549 | Lung | NSCL | ATCC | RPMI (10% FBS) |
| A2058 | Skin | Melanoma | ATCC | DMEM (10% FBS) |
| SK- | Skin | Melanoma | ATCC | MEM (10% FBS) |
| MEL-2 | | | | |
| C33A | Cervical | Carcinoma | ATCC | MEM (10% FBS) |
| SiHa | Cervical | Carcinoma | ATCC | MEM (10% FBS) |
| SK-OV-3 | Ovarian | Carcinoma | ATCC | RPMI (10% FBS) |
| OVCAR-3 | Ovarian | Carcinoma | ATCC | RPMI (10% FBS) |
| SK- | Liver | Hepatocarcinoma | ATCC | MEM (10% FBS) |
| HEP-1 | | | | |
| Hep G2 | Liver | Hepatocarcinoma | ATCC | MEM (10% FBS) |
| Het-1A | Esophagus | Normal | ATCC | BEBM (rhEGF, |
| | | | | 10% FBS) |
| THLE-3 | Liver | Normal | ATCC | BEBM (rhEGF, |
| | | | | 10% FBS) |

ATCC: American Type Culture Collection.

### (2) Test Substance:

A stock of 100 mM in sterilized Dulbecco phosphate buffered saline (DPBS, pH 7.4) was made for CMX001 and cidofovir and stored at −20° C. Doxorubicin, Docetaxel, Oxaliplatin, DTIC (Dacarbazine) and Cisplatin were purchased from Sigma-Aldrich (St. Louis, Mo.). Gemcitabine was manufactured by Eli Lilly and Company and purchased from Infusion Solutions Pharmacy (Tucson, Ariz.).

### (3) Experimental Procedure:

In vitro cell proliferation assays were performed using a 96-well plate based MTSassay (CellTiter 96° AqueousOne Solution Cell Proliferation Assay, Promega) or SRB (Sulforhodamine-B) assay (1-3). Briefly, cells were plated in 100 μl medium on day 0 in 96-well microtiter plates (Falcon #3072). On day 1, 10 μl of serial dilutions of each compound (Nine 1:2 dilutions with a starting concentration of 100 μM; in some cases, a starting concentration of 400 μM (CMX001 or Cidofovir) or 1 μM (some standard agents)) were added in triplicates to the plates. For standard agents using DMSO as solvent, serial dilutions of DMSO with a starting concentration of 0.5% were also included as a vehicle control.

After incubation for 3 days at 37° C. in a humidified incubator, viable cells were measured either by MTS or SRB assay. For MTS assay, 20 μl of a 20:1 mixture of (3-(4,5-dimethylthiazol-2-yl)-5-(3-carboxymethoxyphenyl)-2-(4-sulfophenyl)-2H-tetrazolium, inner salt (MTS, 2 mg/ml) and an electron coupling reagent, phenazine methosulfate (PMS, 0.92 mg/ml in DPBS), was added to each well and incubated for 3 hours at 37° C. Absorbance was measured using a BioTek Synergy™ HT Multiple Detection microplate reader at 490 nm. For SRB assay, cells were fixed in situ with 50 μL of cold trichloroacetic acid (TCA, final concentration at 10% w/v) for 60 minutes. Cells were then washed 5 times with H₂O and air-dried.

Next, cells were stained with 20 μL of 0.2% SRB in 1% acetic acid and incubated at room temperature for 30 minutes and unbound SRB was removed by washing 5 times with 1% acetic acid and air-dried. Finally, bound SRB stain was solubilized in 50 μL of 50 μM Tris buffer before taking an optical density measurement at 570 nm using the BioTek microplate reader. The growth inhibition data were expressed as the percentage of cell survival calculated from the background corrected absorbance. The surviving fraction of cells was determined by dividing the mean absorbance values of the test agent-treated samples by the mean absorbance values of untreated control.

### (4) Statistical Analysis:

Raw data were acquired using a BioTek Synergy™ HT Multiple Detection microplate reader and imported into Microsoft Excel for analysis. The average signal of each treatment was calculated from the triplicates after subtracting the blank signals. The percentage survival values were calculated using the following formula: % survival=(average signal of the drug treatment/average signal of untreated controls)×100. The data were finally imported into the Prism 4 software (GraphPad Software, San Diego, Calif.) to generate the dose-activity curves using non-linear regression curve fit and to calculate the IC₅₀ values.

### B. Results and Discussions

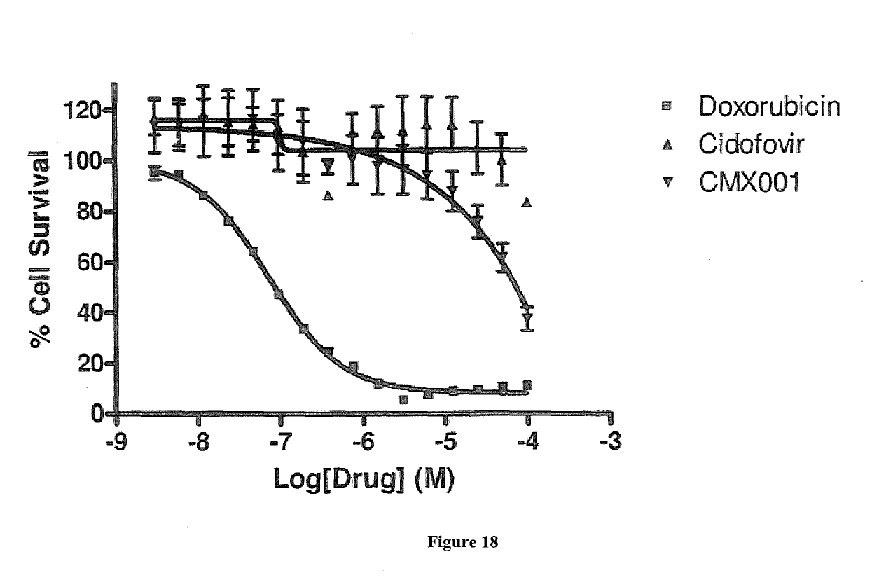

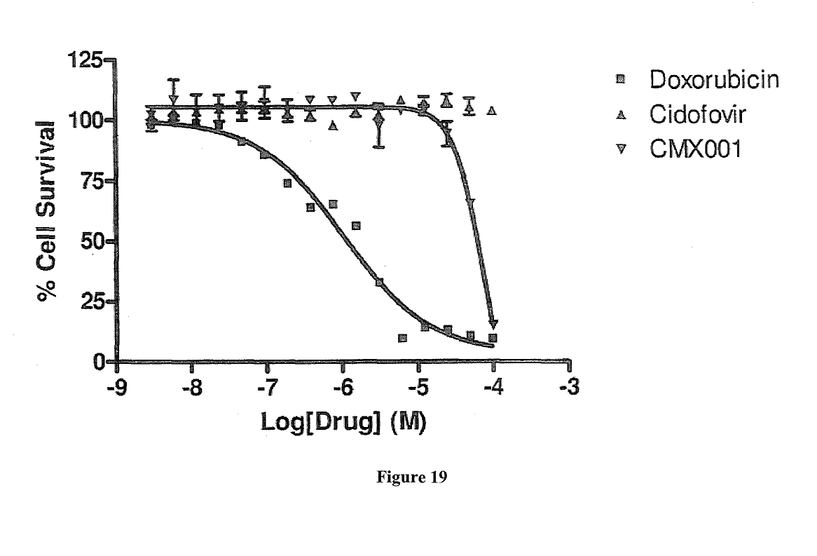

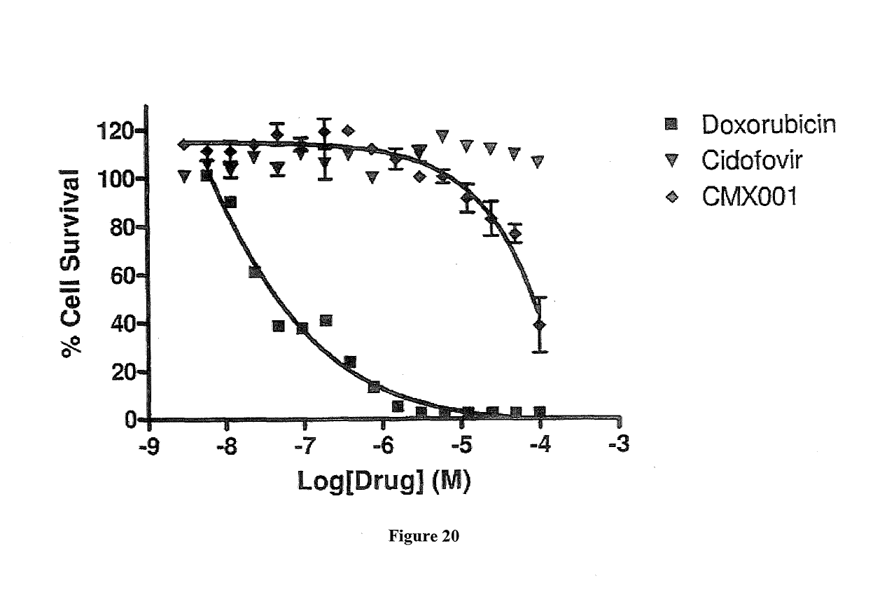

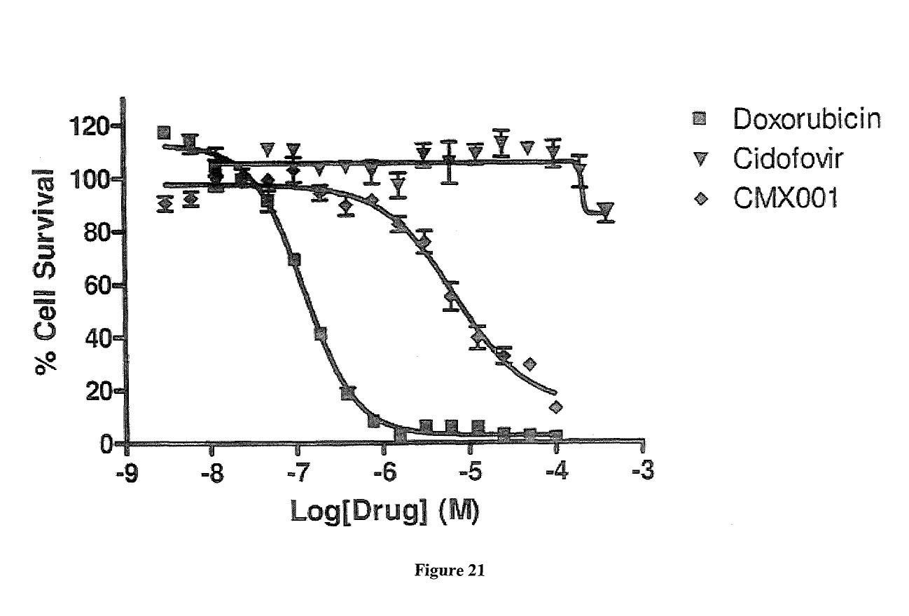

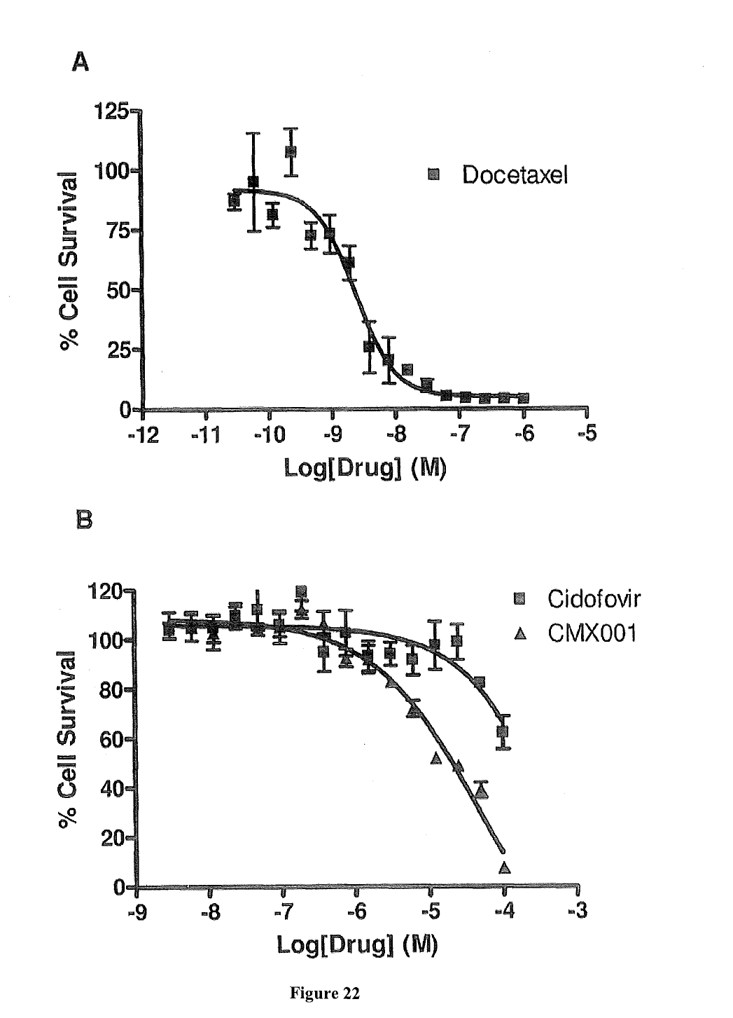

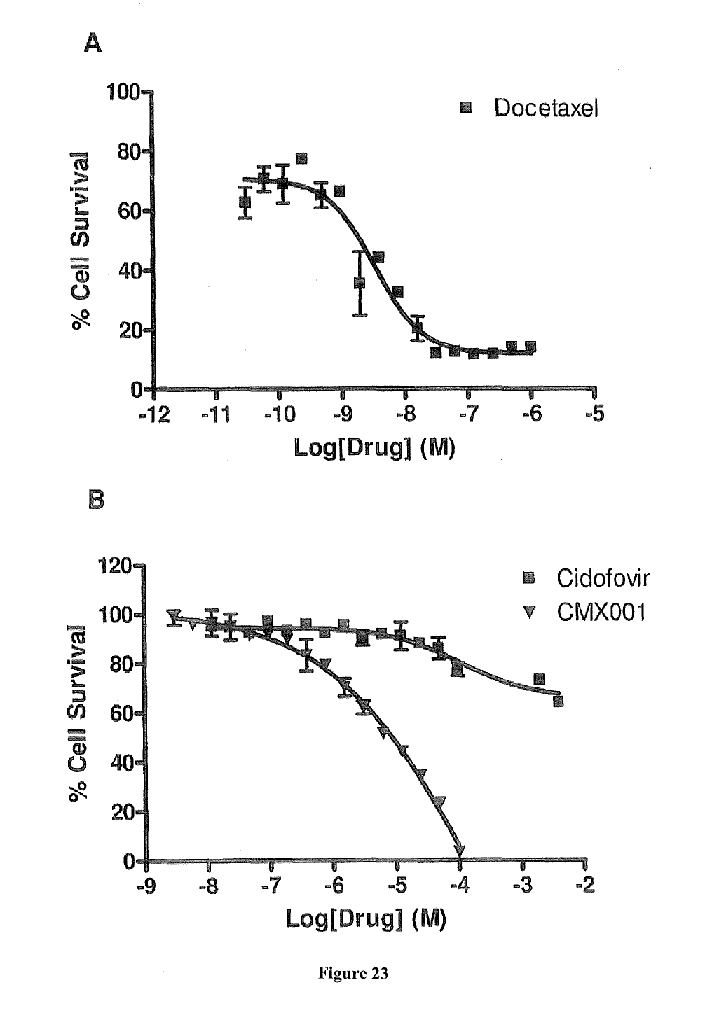

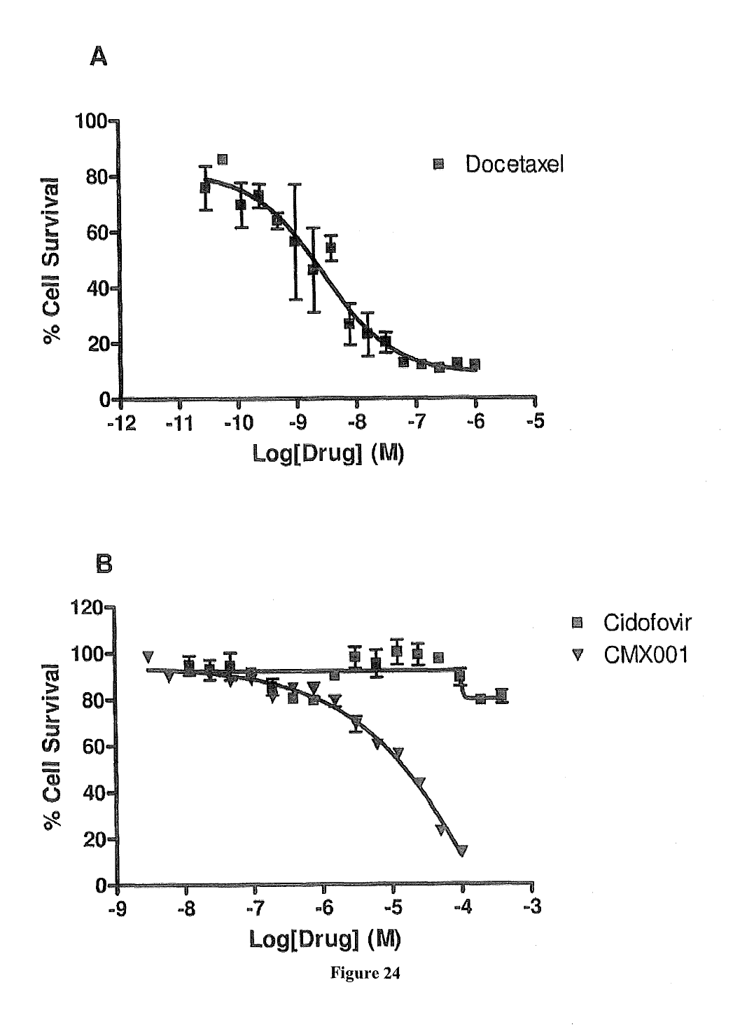

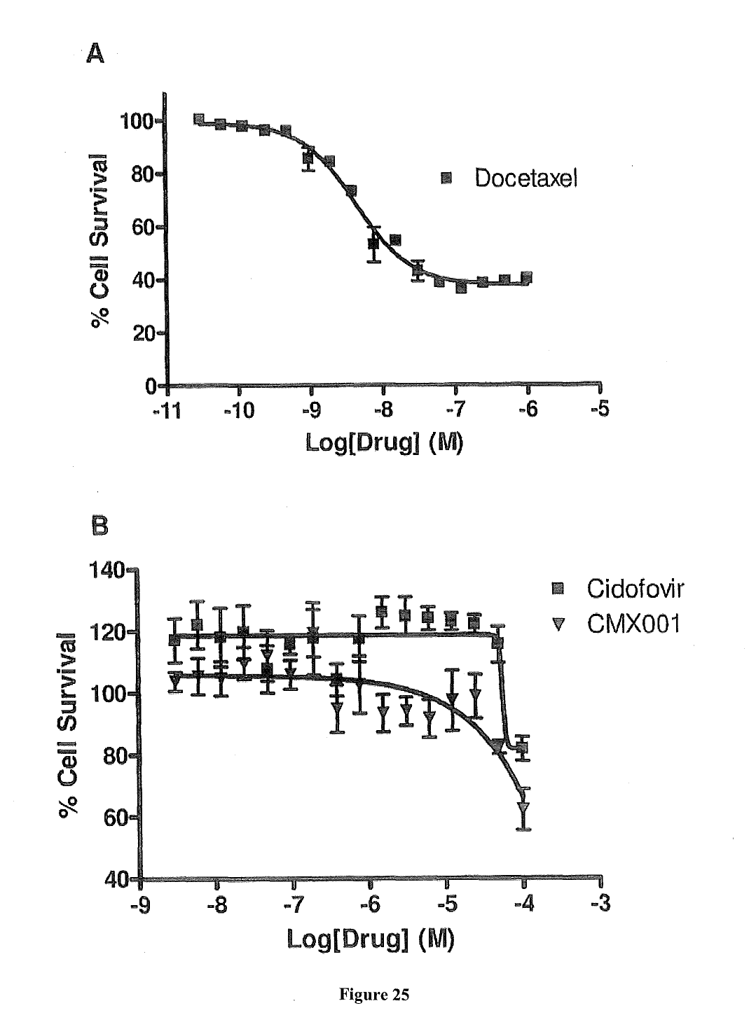

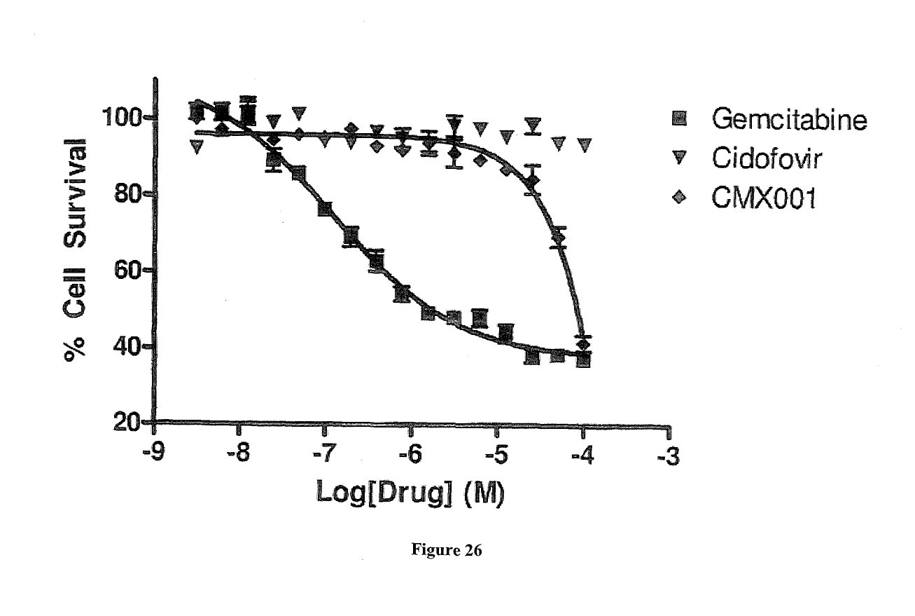

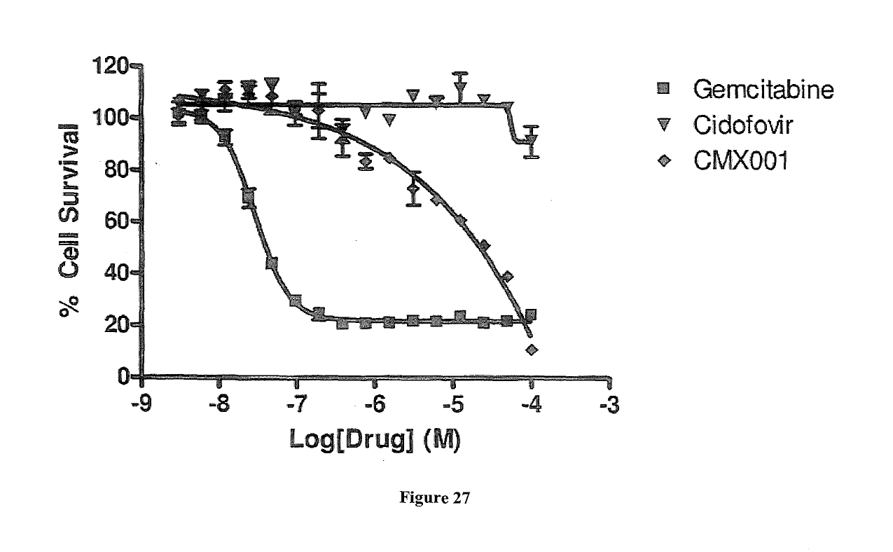

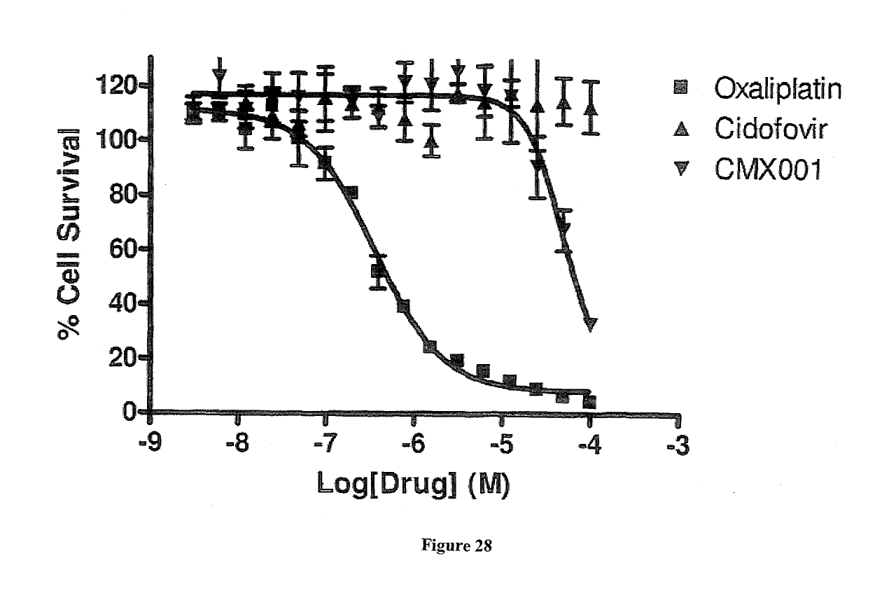

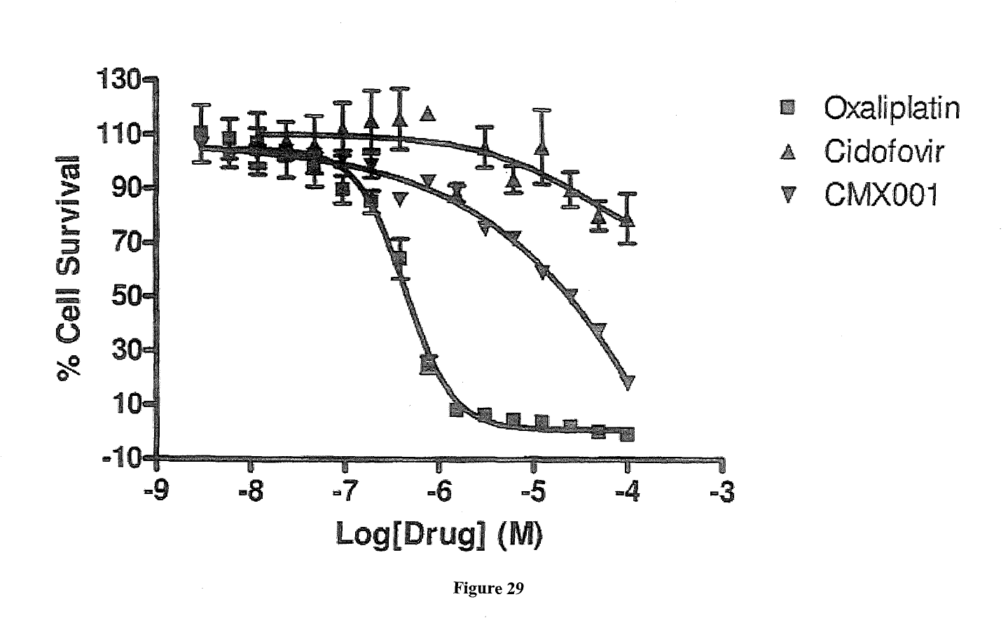

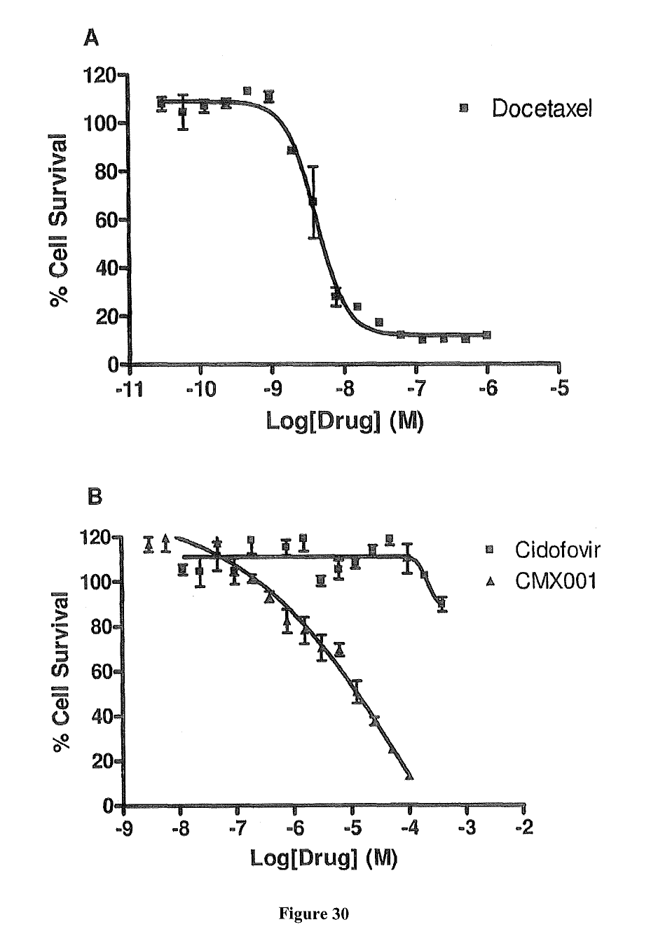

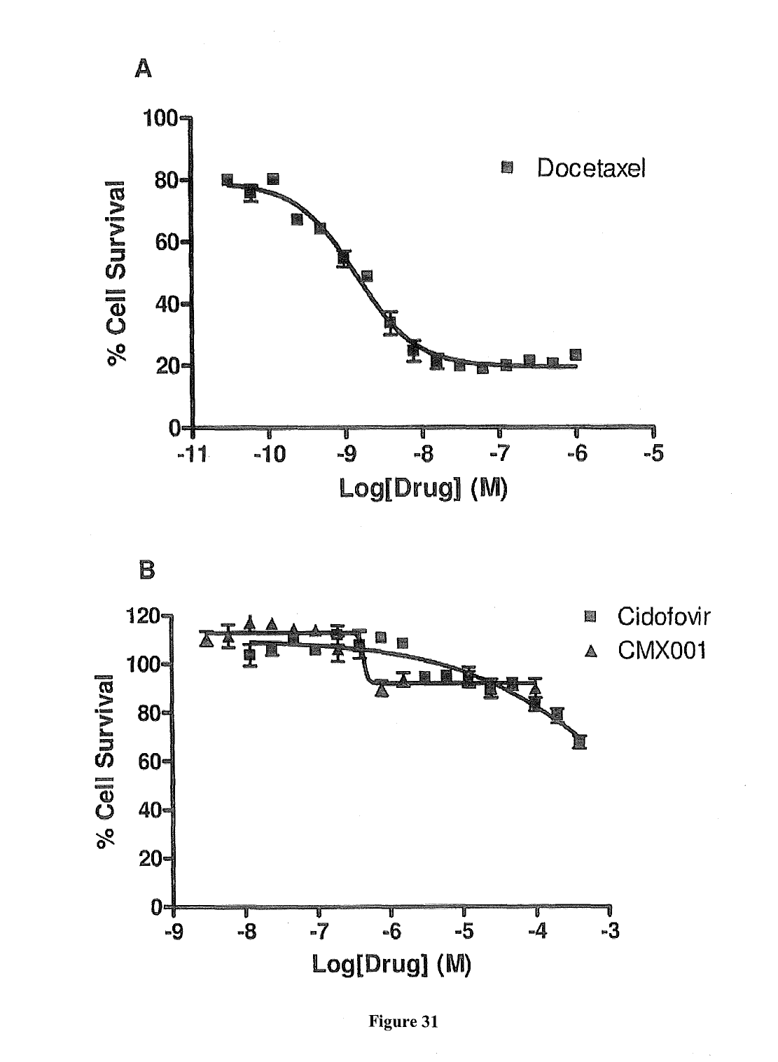

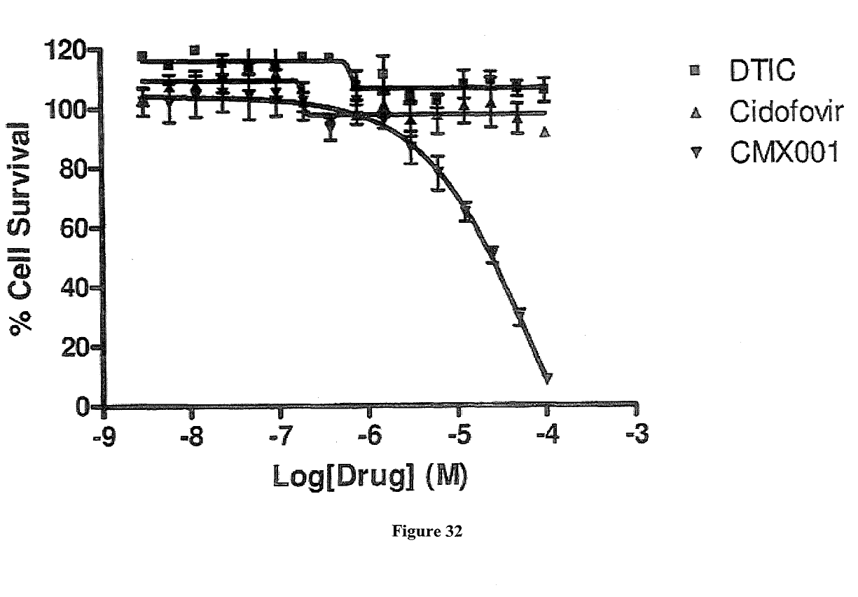

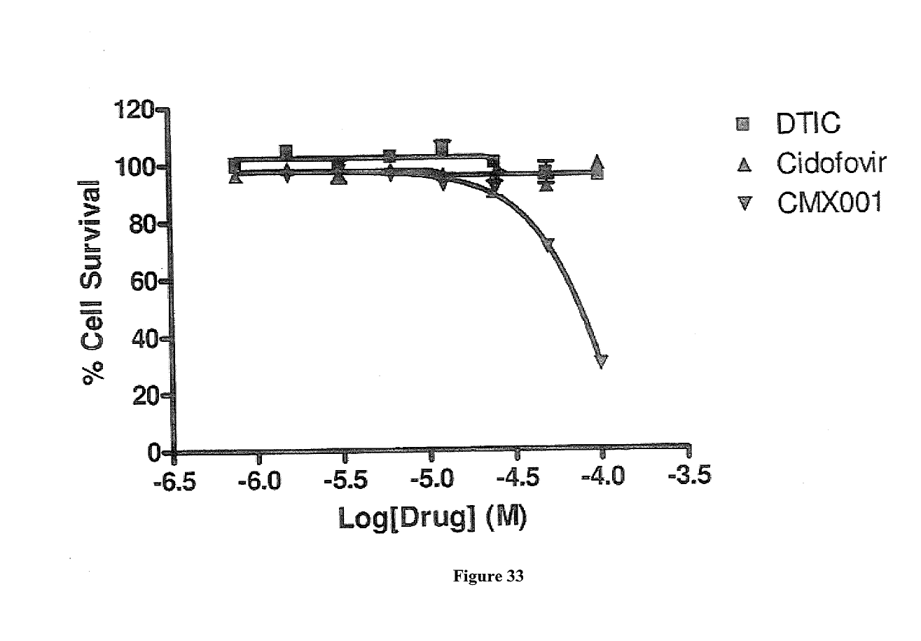

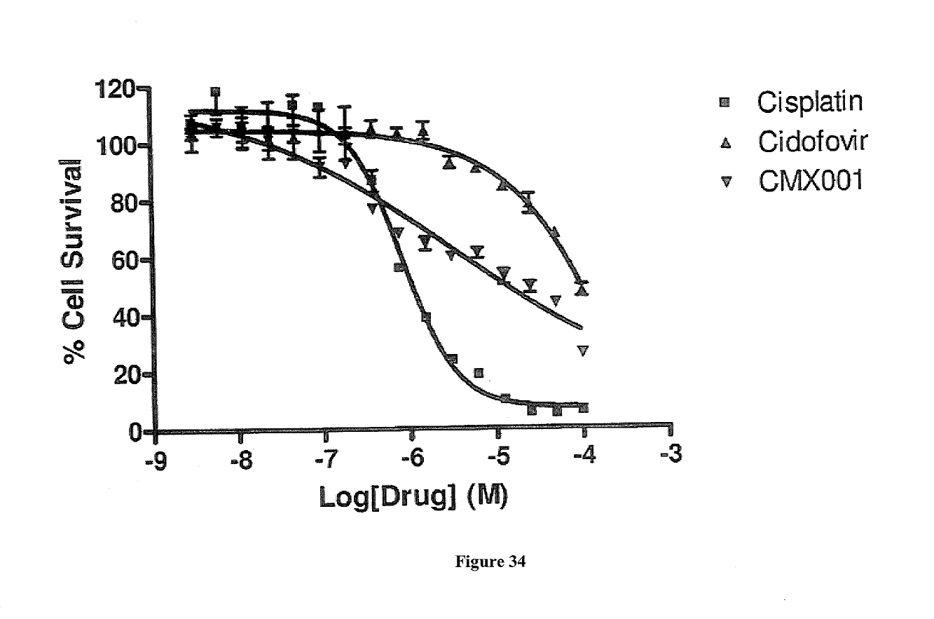

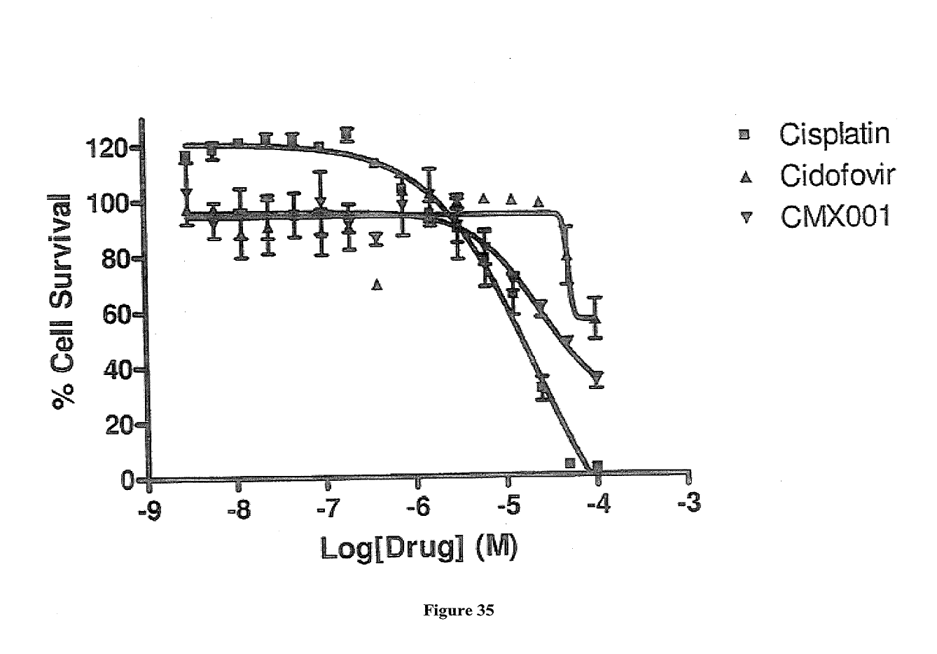

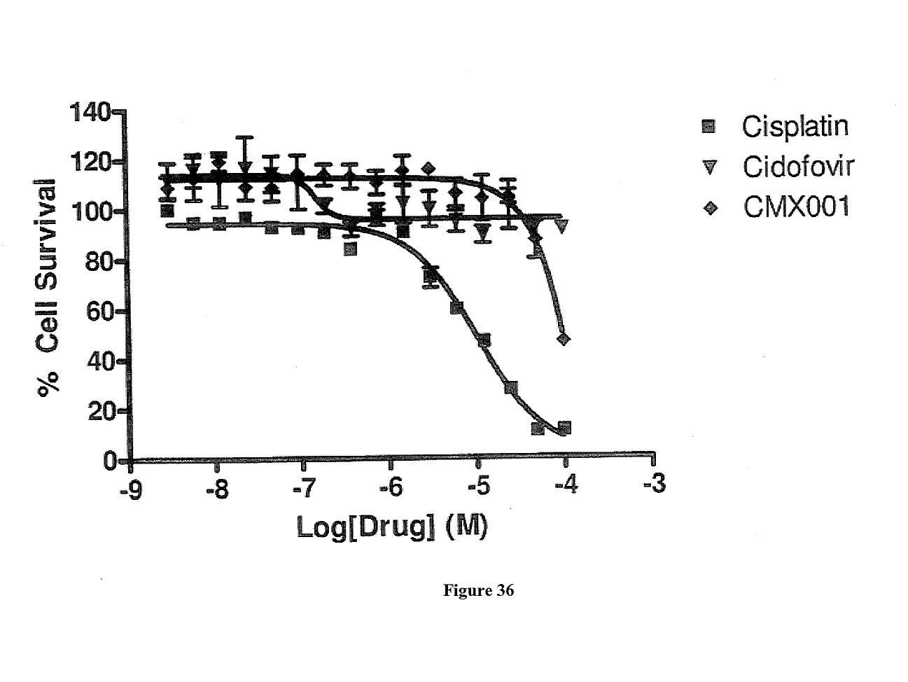

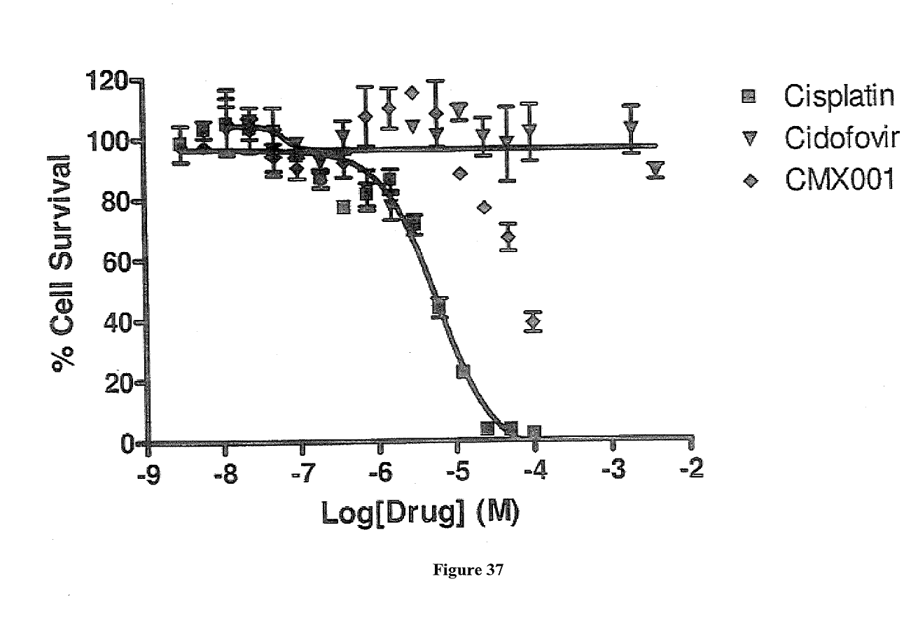

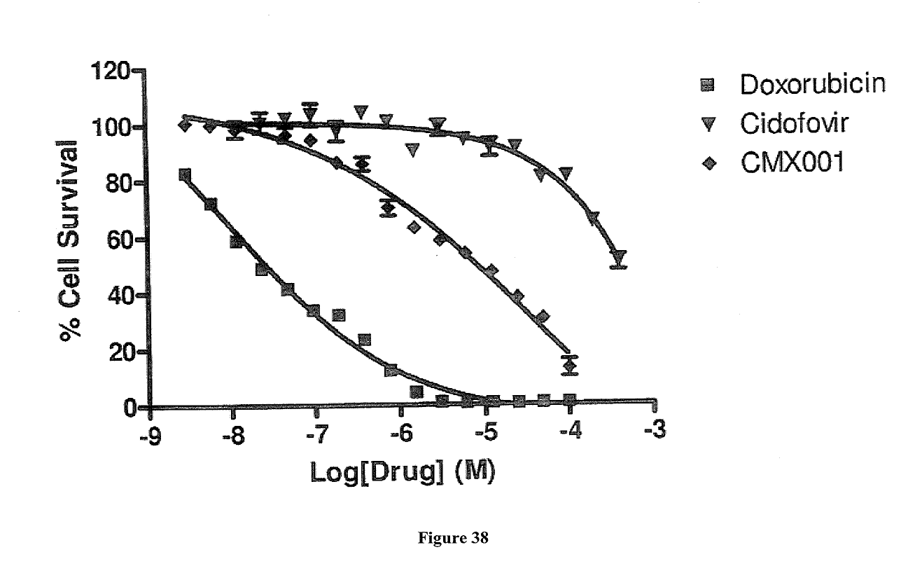

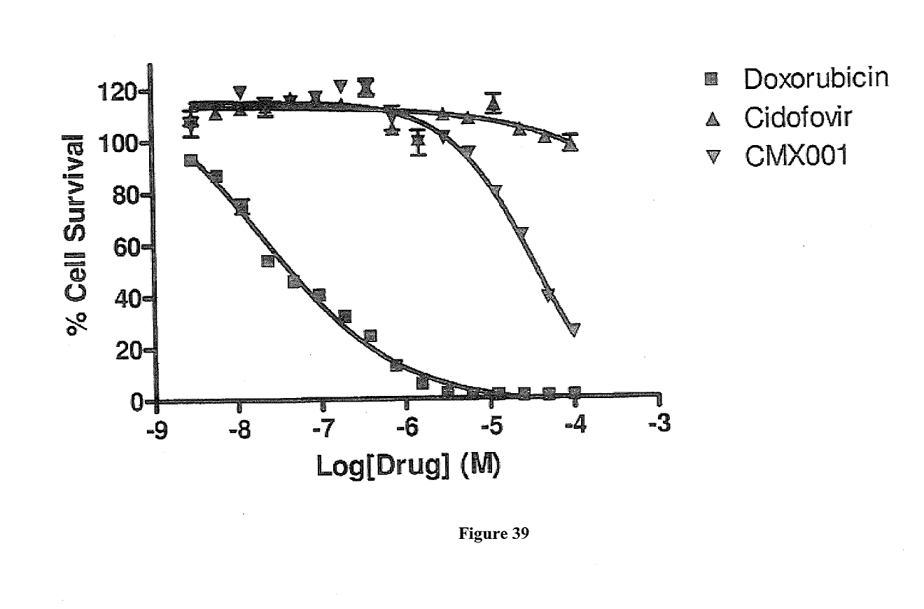

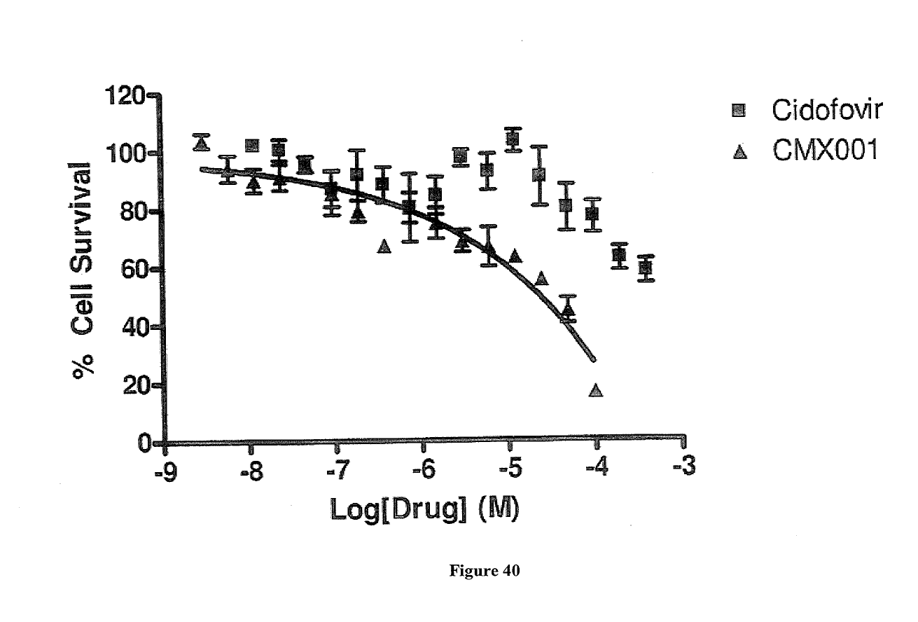

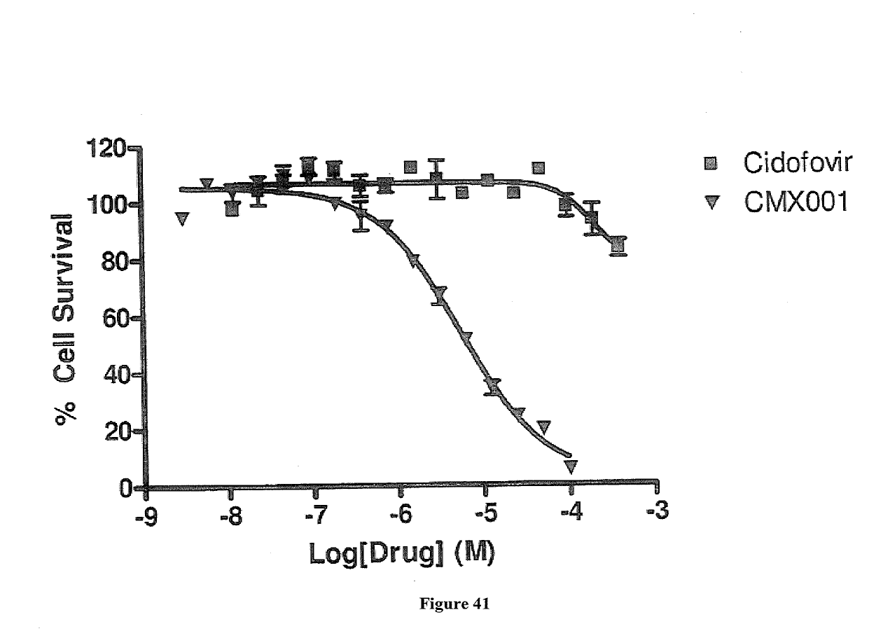

The growth inhibition profiles of the CMX001, Cidofovir, and standard agents in each cell line are shown in FIGS. 18-41. The IC₅₀ (concentration required to achieve 50% growth values are listed in Table 4.

TABLE 4
Estimated IC₅₀ values for CMX001,
Cidofovir and the standard agents

| | | | IC₅₀ for | IC₅₀ for | IC₅₀ for |
|---|---|---|---|---|---|
| Cell | Tissue | Standard | Std Agent | Cidofovir | CMX001 |
| line | Type | Agent | (μM) | (μM) | (μM) |
| Caki-1 | Kidney | Doxorubicin | 0.074 | >100 | 68.6 |
| Caki-2 | Kidney | Doxorubicin | 1.23 | >100 | 60.0 |
| SCaBER | Bladder | Doxorubicin | 0.060 | >100 | 82.1 |
| SW 780 | Bladder | Doxorubicin | 0.16 | >100 | 10.0 |
| DU 145 | Prostate | Docetaxel | 0.0024 | >100 | 19.0 |
| PC-3 | Prostate | Docetaxel | 0.0011 | >100 | 6.6 |
| MDA-231 | Breast | Docetaxel | 0.0028 | >100 | 11.2 |
| MCF-7 | Breast | Docetaxel | 0.015 | >100 | 62.0 |
| PANC-1 | Pancre- | Gemcitabine | 0.13 | >100 | 91.5 |
| | atic | | | | |
| MIA | Pancre- | Gemcitabine | 0.029 | >100 | 14.6 |
| PaCa-2 | atic | | | | |
| HT-29 | Colon | Oxaliplatin | 0.36 | >100 | 48.6 |
| HCT-116 | Colon | Oxaliplatin | 0.48 | >100 | 20.9 |
| H460 | Lung | Docetaxel | 0.0042 | >100 | 12.4 |
| A549 | Lung | Docetaxel | 0.0014 | >100 | >100 |
| A2058 | Skin | DTIC | >100 | >100 | 21.7 |
| SK- | Skin | DTIC | >100 | >100 | 70.5 |
| MEL-2 | | | | | |
| C33A | Cervical | Cisplatin | 1.2 | 96.7 | 14.3 |
| SiHa | Cervical | Cisplatin | 15.5 | 100 | 43.1 |
| SK-OV-3 | Ovarian | Cisplatin | 9.2 | >100 | 95.5 |
| OVCAR-3 | Ovarian | Cisplatin | 4.9 | >100 | 73.3 |
| SK- | Liver | Doxorubicin | 0.029 | >100 | 7.4 |
| HEP-1 | | | | | |
| Hep G2 | Liver | Doxorubicin | 0.050 | >100 | 37.9 |
| Het-1A | Esoph- | — | — | >100 | 20.5 |
| | agus | | | | |
| | (Normal) | | | | |
| THLE-3 | Liver | — | — | >100 | 7.0 |
| | (Normal) | | | | |

In summary, CMX001 was more potent than its parent compound, Cidofovir, in almost all 24 cell lines evaluated in the study. The IC₅₀ values of CMX001 in the tumor cell lines ranged from 6.6 μM to >100 μM, which are relatively moderate when compared to those of the standard agents. Among the 22 tumor cell lines tested, CMX001 was most active in PC-3 (IC₅₀=6.6 μM) and SK-HEP-1 (IC₅₀=7.4 μM). In general, CMX001 was most active in prostate, liver and cervical cancer cell lines, and was least active in kidney, bladder and ovarian cancer cell lines. Interestingly, CMX001 was also fairly active against two immortalized human normal epithelial cell lines, Het-1A (esophagus) and THLE-3 (liver), with an IC₅₀ of 20.5 μM and 7.0 μM, respectively. Both cell lines were immortalized by the SV40 large T-antigen virus (4-5).

DTIC (dacarbazine) was not active against the melanoma cell lines (IC₅₀>100 μM). One explanation for this low activity of DTIC in cell proliferation assay is that DTIC requires hepatic activation for its antitumor activity. However, temozolomide, an analog of DTIC which does not require hepatic activation, did not show good activity in these two cell lines either (IC₅₀>100 μM, data not shown). Therefore, it could be merely that these two cell lines are resistant to DTIC and temozolomide, as has been shown in some other melanoma cell lines (6-7). The fact that CMX001 showed some activity in these two melanoma cell lines is potentially encouraging. It may be worthwhile to consider studies that look at the influence of duration of exposure with activity. In this regard, longer exposure times may result in enhanced cytotoxicity.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed and that modifications to the disclosed embodiments, as well as other embodiments, are intended to be included within the scope of the appended claims. The invention is defined by the following claims, with equivalents of the claims to be included therein.

1-63. (canceled)

64. A method of treating a viral induced tumor in a subject, the method comprising administering to said subject a therapeutically effective amount of a compound selected from the group consisting of 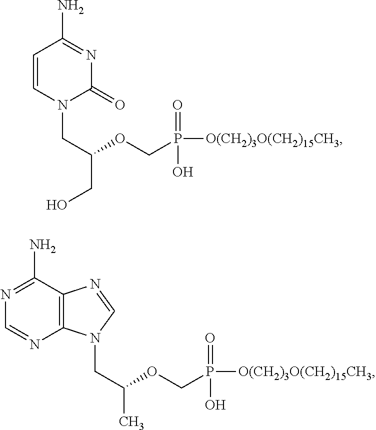
and a pharmaceutically acceptable salt thereof, such that the viral induced tumor is treated.

65. The method of claim 64, wherein the compound targets viral infected cells and/or viral replications.

66. The method of claim 65, wherein the viral infected cells are prostate adenocarcinoma cells, breast adenocarcinoma cells, or colon adenocarcinoma cells.

67. The method of claim 64, wherein the viral induced tumor is adenocarcinoma.

68. The method of claim 64, wherein the viral induced tumor is a prostate, liver, cervix, breast, or colon tumor, or hepatocellular carcinoma.

69. The method of claim 64, wherein the subject is human.

70. The method of claim 64, wherein the viral induced tumor is associated with polyomavirus, papillomavirus, herpes virus, adenovirus, Epstein-Barr virus (EBV), human cytomegalovirus (HCMV), Hepatitis B virus, Hepatitis C virus or a combination thereof.

71. The method of claim 64, wherein the viral induced tumor is associated with papillomavirus selected from the group consisting of human papillomavirus, cottontail rabbit papillomavirus, equine papillomavirus and bovine papillomavirus; cervical cancer associated with human papillomavirus; post transplant lymphoproliferative disorder (PTLD) associated with Epstein-Barr virus (EBV); or glioblastoma associated with cytomegalovirus.

72. The method of claim 64, wherein the viral induced tumor is associated with polyomavirus selected from the group consisting of BK virus, John Cunningham (JC) virus, Merkel cell virus (MCV), KI polyomavirus (KN), WU polyomavirus (WUV), and Simian virus 40 (SV 40).

73. The method of claim 72, wherein the viral induced tumor is associated with BK virus or JC virus.

74. The method of claim 64, wherein the subject is immunocompromised.

75. The method of claim 74, wherein the subject is a transplant patient on immunosuppressive medications.

76. The method of claim 74, wherein the subject is infected with HIV.

77. The method of claim 64, further comprising administering to the subject at least one immunosuppressant agent.

78. The method of claim 77, wherein the immunosuppressant agent is concurrently or sequentially administered to the subject.

79. The method of claim 77, wherein the immunosuppressant agent is selected from the group consisting of Daclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm and combinations thereof.

80. The method of claim 64, wherein the compound is administered at a dose of about 50 mg, about 75 mg, about 100 mg, about 150 mg, about 200 mg, about 250 mg, about 300 mg or about 400 mg.

81. The method of claim 64, wherein the compound is administered at a dose of about 200 mg or about 400 mg.

82. A method of treating a polyomavirus infection in a subject, the method comprising administering to said subject a therapeutically effective amount of a compound selected from the group consisting of 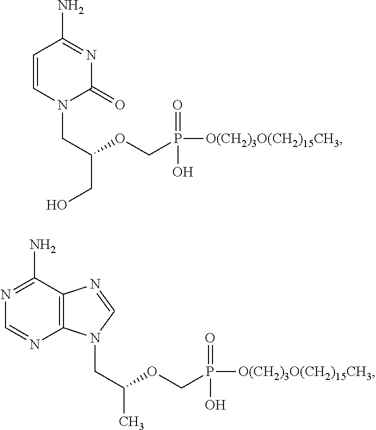
and a pharmaceutically acceptable salt thereof;
wherein the compound is administered at a dose of about 50 mg, about 75 mg, about 100 mg, about 150 mg, about 200 mg, about 250 mg, about 300 mg or about 400 mg, such that the polyomavirus infection is treated.

83. The method of claim 82, wherein the polyomavirus is BK virus or JC virus.
